(19) United States
(12) Patent Application Publication
(10) Pub. No.: US 2015/0051161 A1
(43) Pub. Date: Feb. 19, 2015

(54) Methods for the Detection of Advanced Glycation Endproducts and Markers for Disease

(71) Applicant: THE JOHNS HOPKINS UNIVERSITY, Baltimore, MD (US)

(72) Inventors: Richard David Semba, Baltimore, MD (US); Luigi Ferruci, Baltimore, MD (US); Edward G. Lakatta, Bethesda, MD (US)

(73) Assignee: THE JOHNS HOPKINS UNIVERSITY, Baltimore, MD (US)

(21) Appl. No.: 14/249,451

(22) Filed: Apr. 10, 2014

Related U.S. Application Data
(63) Continuation of application No. 13/002,106, filed on Jul. 11, 2011, now abandoned.
(60) Provisional application No. 61/133,510, filed on Jun. 30, 2008.

(51) Int. Cl. G01N 33/53; A61K 31/426; A61K 31/60; A61K 31/40; A61K 31/498; A61K 31/496; A61K 31/155; A61K 38/05

(52) U.S. Cl. CPC G01N 33/5308; A61K 31/155; A61K 31/426; A61K 38/05; A61K 31/40; A61K 31/498; A61K 31/496; A61K 31/60; G01N 2800/50; G01N 2800/00
USPC 514/21.91; 514/634; 514/365; 514/424; 514/250; 514/369; 514/252.13; 514/165; 435/7.92

(57) ABSTRACT

The present invention provides compositions and methods for detecting carboxymethyl-lysine (CML) and circulating receptor for advanced glycation end (RAGE) products, and methods for correlating CML and RAGE levels with age-related disease. In particular, serum CML and/or circulating receptor for advanced glycation end (RAGE) products can be used as a clinical biomarker in diagnostics to identify people who are at a higher risk of developing adverse ageing-related outcomes.

### CROSS-REFERENCE TO RELATED APPLICATION

This application claims the benefit of U.S. Provisional Application No. 61/133,510, which was filed Jun. 30, 2008, the entire contents of which are incorporated herein by reference.

### GOVERNMENT SUPPORT

This work was supported by the National Institutes of Health. The Government has certain rights in this application.

### BACKGROUND OF THE INVENTION

The factors that increase the risk of common adverse aging-related outcomes, such as cardiovascular disease, chronic kidney disease, loss of muscle strength, and anemia have not been completely characterized. Advanced glycation end products (AGEs) are bioactive molecules that are formed by the non-enzymatic glycation of proteins, lipids, and nucleic acids. AGEs have been implicated in the pathogenesis of diabetes, atherosclerosis, and renal disease. AGEs induce covalent cross-links with proteins such as collagen, contribute to arteriosclerosis and atherosclerosis, and increased glomerular sclerosis and interstitial fibrosis in the kidney. AGEs upregulate inflammation through receptor for AGEs (RAGE) and endogenous secretory receptor for AGEs (esRAGE) Carboxymethyl-lysine (CML) is a dominant AGE that is found in serum and tissues. Compositions and methods for detecting CML and correlating CML with disease are required.

### SUMMARY OF THE INVENTION

As described below, the present invention generally provides compositions and methods for detecting carboxymethyl-lysine (CML) and circulating receptor for advanced glycation end (RAGE) products, and methods for correlating CML and RAGE levels with age-related disease.

In particular, serum CML and/or circulating receptor for advanced glycation end (RAGE) products can be used as a clinical biomarker in diagnostics to identify people who are at a higher risk of developing adverse aging-related outcomes. In other embodiments, serum CML and/or circulating receptor for advanced glycation end (RAGE) products can be used as a clinical biomarker or test to identify people who are at a higher risk of developing adverse aging-related outcomes. The invention features, for example, an enzyme-linked immunosorbent assay (ELISA) or similar ELISA-based assay that can be used for quantification of serum CML.

The invention provides methods of diagnosing a subject as having, or having a propensity to develop an ageing related disease or disorder, the method including detecting carboxymethyl lysine (CML) in a subject sample, wherein an alteration in the level of CML relative to the level in a reference control sample indicates that the subject is prone to or has or has a propensity to develop an ageing related disease or disorder. Methods provided by the invention can further include determining the level of one or more receptors for advanced glycation endproducts in the sample. In certain embodiments, ageing related diseases include, but are not limited to, reduced kidney function, renal insufficiency, skeletal muscle strength, sarcopenia, cardiovascular disease, cardiovascular disease-related death, and anemia. In certain embodiments, renal insufficiency or reduced kidney function is defined as having a reduced glomerular filtration rate (GFR). In certain embodiments of the invention, level of CML is determined, for example, in an immunological assay. Samples for assaying in the methods of the invention include, but are not limited to, a serum sample, a fasting blood sample, and a non-fasting blood sample.

The invention provides methods for determining a propensity to developing a certain age related disease or disorder in specific subsets of subjects. In certain embodiments, the subject is a human female. In certain embodiments, the subject has certain other diseases, conditions, or habits in addition to an elevated advanced glycosylation end products (AGE), for example elevated CML, total receptor (R) for AGE, circulating sRAGE, or endogenous secretory (es)RAGE. For example, the subject can have one or more of increased alcohol intake, be a smoker, have an increased body mass index, have elevated fasting plasma glucose, have elevated mean arterial pressure, have elevated serum triglycerides, have elevated high density lipoproteins (HDL), have elevated low density lipoproteins (LDL), have elevated C-reactive protein, have a low mini-mental status exam score, have hypertension, have coronary artery disease, have congestive heart failure, have peripheral artery disease, have or had a stroke, have diabetes mellitus, have cancer, or have renal insufficiency. The invention further provides methods for detection of one or more of the conditions or habits in a subject in addition to the presence of AGE, sRAGE, or esRAGE. The invention provides methods further including the detection of serum carotenoids. Increased serum CML or other AGE, in conjunction with low serum carotenoids are associated with a subject having or having a propensity to develop poor grip strength and/or sarcopenia.

The data provided in the Tables herein provide confidence intervals and p-values defining the strength of the propensity of a subject to develop a certain age related disease or condition. Provided with the specific conditions and laboratory values provided herein, one of skill in the art can select specific combinations of habits or conditions that have a stronger propensity to develop certain age related diseases (e.g., alcohol intake, be a smoker, have an increased body mass index, have elevated fasting plasma glucose, have elevated mean arterial pressure, have elevated serum triglycerides, have elevated C-reactive protein, have elevated high density lipoproteins (HDL), have elevated low density lipoproteins (LDL), have a low mini-mental status exam score, have hypertension, have coronary artery disease, have congestive heart failure, have peripheral artery disease, have or had a stroke, have diabetes mellitus, have cancer, or have renal insufficiency). The invention also provides methods for selection of subsets of subjects based on the presence of one or more of the habits or one or more of the conditions as having an even greater propensity to specific ageing related conditions. Such selections can be readily made by those of skill in the art. Similarly, the invention provides methods for selection of subsets of subjects wherein the presence of one or more of the habits or one or more of the conditions explicitly need not be considered when determining a propensity to a specific ageing related disease.

The invention provides methods for determining a propensity to have an increased risk of cardiovascular death by a subject by the detection of increased circulatory RAGE as compared to control identifies the subject, for example by detection of circulatory RAGE comprises measuring total sRAGE and esRAGE.

The invention provides methods for determining a propensity to have an increased risk of developing renal insufficiency by a subject by the detection of increased serum CML as compared to control identifies the subject.

The invention provides methods for determining a propensity to have an increased risk of developing anemia by a subject by the detection of increased serum serum CML, sRAGE, and esRAGE as compared to control identifies the subject.

The invention provides methods of treating or preventing an ageing related disease or disorder in a subject by administering to a subject in need thereof an effective amount of a composition that reduces the risk associated with an increased level of CML or one or more receptors for advanced glycation endproducts. In certain embodiments, the composition is an AGE-breaker or AGE inhibitor. In certain embodiments, the methods of prevention and treatment include imposing on the subject dietary restriction of AGE-containing foods, for example foods processed at high temperatures, deep fried, oven fried, grilled, or broiled. In certain embodiments, the invention can further include increasing carotenoid intake in the subject. The treatment and prevention methods provided herein can be used for the treatment and prevention of conditions including, but not limited to, reduced kidney function, renal insufficiency, skeletal muscle strength, sarcopenia, cardiovascular disease, cardiovascular disease-related death, and anemia.

The invention provides methods for monitoring subjects having an ageing related disease by detecting carboxymethyl lysine (CML) in a subject sample, wherein an alteration in the level of CML relative to the level in a control sample indicates a change in the ageing related disease or disorder. For example, an increase in CML can be indicative that the subject has or has a propensity to develop an ageing related disease or disorder. A decrease in CML can be indicative of an amelioration of the ageing related disease. Monitoring methods can further include determining the level of one or more receptors for advanced glycation endproducts in the sample. Diseases to be monitored by the methods of the invention include, but are not limited to ageing related diseases such as reduced kidney function, renal insufficiency, skeletal muscle strength, sarcopenia, cardiovascular disease, cardiovascular disease-related death, and anemia. In certain embodiments of the invention, level of CML is determined, for example, in an immunological assay. Samples for assaying in the methods of the invention include, but are not limited to, a serum sample, a fasting blood sample, and a non-fasting blood sample.

The monitoring methods provided by the invention include detection of one or more an analytes including increased serum CML, increased RAGE expression and increased circulating RAGE. The monitoring methods of the invention can be used, for example, to monitor efficacy or compliance with dietary restriction and/or the efficacy of AGE breakers or AGE inhibitors. In certain embodiments, no reduction in CML levels after dietary restriction is indicative of a need for treatment with an AGE breaker or AGE inhibitor.

The invention provides kits for practicing the diagnostic and monitoring methods of the invention. For example a kit for the diagnosis of an ageing-related disease in a subject can include a composition for detecting CML in a sample and directions for use of the kit. In certain embodiments, the antibody that detects CML in an immunological assay. In certain embodiments, the kit includes one or more further reagents for the detection of the presence of sRAGE or esRAGE in a sample.

The invention further provides method of selecting a treatment regimen for a subject having, or having a propensity to develop an ageing related disease or disorder, the method comprising detecting carboxymethyl lysine (CML) in a subject sample, wherein an increase in the level of CML relative to the level in a control sample indicates that the subject should be treated to reduce AGE or RAGE levels. In certain embodiments, the method also includes determining the level of one or more receptors for advanced glycation endproducts in th

[symbol]

Other features and advantages of the invention will be apparent from the detailed description, and from the claims.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 is a Table showing the demographic and health characteristics of women, aged ≧65 years, in the Women's Health and Aging Study I in Baltimore, Md. with and without reduced GFR.

FIG. 2 is a Table showing separate multivariate logistic regression models of the relation of serum CML, sRAGE and esRAGE with reduced GFR in women aged ≧65 years, in the Women's Health and Aging Study I in Baltimore, Md.

FIG. 3 is a Table showing separate multivariate logistic regression models of the relation of serum CML, sRAGE and esRAGE at baseline with estimated GFR in women aged ≧65 years, in the Women's Health and Aging Study I in Baltimore, Md.

FIG. 4 is a Table showing characteristics of women in the Women's Health and Aging Study I (N=559).

FIG. 5 is a Table showing univariate linear regression models of serum carboxymethyl-lysine and other factors with grip strength.

FIG. 6 is a Table showing multivariate linear regression models of serum carboxymethyl-lysine, sRAGE, and esRAGE with grip strength.

FIG. 7 is a Table showing demographic and health characteristics of women, aged ≧65 Years, in the Women's Health and Aging Study I in Baltimore, Md. who survived or died from all causes during follow-up (n=559).

FIG. 8 is a graph showing survival curves for all-cause mortality among women, 65 years, in the Women's Health and Aging Study I in Baltimore, Md., by quartile of serum CML. Women in the highest quartile (quartile 4) of serum CML had lower survival compared to women in the lower three tertiles together (P=0.013, log-rank test).

FIG. 9 is a Table showing demographic and health characteristics of women, aged ≧65 Years, in the women's health and aging study I in Baltimore, Md. who survived or died from cardiovascular diseases during follow-up (n=487).

FIG. 10 is a Table showing multivariate cox proportional hazards models of serum carboxymethyl-lysine and RAGE and all-cause mortality among women aged ≧65 years, in the Women's Health and Aging Study I in Baltimore, Md.

FIG. 11 is a graph showing survival curves for cardiovascular disease mortality among women, 65 years, in the Women's Health and Aging Study I in Baltimore, Md., by quartile of serum CML. Women in the highest quartile (quartile 4) of serum CML had lower survival compared to women in the lower three tertiles together (P=0.0009, log-rank test).

FIG. 12 is a Table showing multivariate Cox proportional hazards models of serum carboxymethyl-lysine and RAGE and cardiovascular disease mortality among women aged >65 years, in the Women's Health and Aging Study I in Baltimore, Md.

FIG. 13 is a Table showing characteristics of men and women with and without anemia in the Baltimore Longitudinal Study of Aging.

FIG. 14 is a Table showing multivariate linear regression models of the relation of serum carboxymethyl-lysine with anemia in men and women in the Baltimore Longitudinal Study of Aging.

FIG. 15 is a Table showing univariate relationships of serum CML, demographic, and disease characteristics with hemoglobin in the Baltimore Longitudinal Study of Aging.

FIG. 16 is a Table showing multivariate linear regression models of the relation of serum carboxymethyl-lysine with hemoglobin in men and women in the Baltimore Longitudinal Study of Aging.

FIG. 17 is a scatterplot of the relationship of serum CML with hemoglobin with Lowess smoothing line.

FIG. 18 is a Table showing demographic and health characteristics of women, aged ≧65 years, in the Women's Health and Aging Study I in Baltimore, Md. with and without anemia.

FIG. 19 is a Table showing multivariate linear regression models of the relation of serum carboxymethyl-lysine, sRAGE, and esRAGE with anemia in women aged ≧65 years, in the Women's Health and Aging Study I in Baltimore, Md.

FIG. 20 is a Table showing multivariate linear regression models of the relation of serum carboxymethyl-lysine, sRAGE, and esRAGE at baseline with hemoglobin in women aged ≧65 years, in the Women's Health and Aging Study I in Baltimore, Md.

FIG. 21 is a Table showing serum CML, sRAGE, and esRAGE, and other characteristics with women without anemia and with specific types of anemia in the Women's Health and Aging Study I in Baltimore, Md.

FIG. 22 is a Table showing demographic and health characteristics of adult men and women with and without renal insufficiency in the Baltimore Longitudinal Study of Aging.

FIG. 23 is a Table showing multivariate linear regression models of the relation of serum carboxymethyl-lysine with chronic renal insufficiency in men and women in the Baltimore Longitudinal Study of aging.

FIG. 24 is a Table showing univariate relationships between serum CML and other factors with estimated glomerular filtration rate in men and women in the Baltimore Longitudinal Study of Aging.

FIG. 25 is a Table showing multivariate linear regression models of the relation of serum carboxymethyl-lysine with estimated glomerular filtration rate in men and women in the Baltimore Longitudinal Study of Aging.

FIG. 26 is a Table showing demographic and health characteristics of women, aged ≧65 years, in the Women's Health and Aging Study I in Baltimore, Md. with and without reduced GFR1.

FIG. 27 is a Table showing separate multivariate logistic regression models of the relation of serum CML, sRAGE, and esRAGE with reduced GFR in women, aged ≧65 years, in the Women's Health and Aging Study I in Baltimore, Md.

FIG. 28 is a Table showing separate multivariate linear regression models of the relation of serum CML, sRAGE, and esRAGE at baseline with estimated GFR in women, aged ≧65 years, in the Women's Health and Aging Study I in Baltimore, Md.

FIG. 29 is a Table showing characteristics of study subjects in the Baltimore Longitudinal Study of Aging with aortic pulse wave velocity and serum carboxymethyl-lysine measurements.

FIG. 30 is a Table showing univariate relationships of demographic, disease, serum carboxymethyl-lysine, and other factors with aortic pulse wave velocity in 493 adults in the Baltimore Longitudinal Study of Aging.

FIG. 31 is a bar graph showing the geometric mean aortic pulse wave velocity (PWV) by tertile of serum CML in adults in the Baltimore Longitudinal Study of Aging. Bars indicate 95% confidence intervals. P=0.01 by ANOVA.

FIG. 32 is a Table showing multivariate linear regression models for serum carboxymethyl-lysine and other risk factors associated with aortic pulse wave velocity (PWV).

FIG. 33 is a Table showing demographic and health characteristics of men and women, aged 65 years, with and without chronic kidney disease at enrollment in the InCHIANTI study.

FIG. 34 is a Table showing multivariate logistic regression models of the relation of plasma CML with prevalent chronic kidney disease in adults, aged 65 years, at enrollment in the InCHIANTI study.

FIG. 35 is a Table showing univariate relationships between plasma CML and other factors with estimated glomerular filtration rate in adults, aged ≧65 years, at enrollment in the InCHIANTI study.

FIG. 36 is a Table showing separate multivariate linear regression models of the cross-sectional relationship of plasma CML with estimated glomerular filtration rate in adults, aged 65 years, at enrollment in the InCHIANTI study.

FIG. 37 is a Table showing separate multivariate linear regression models of the relationship of plasma CML at enrollment with estimated glomerular filtration rate in adults, aged 65 years, at 3 and 6 years of follow-up in the InCHIANTI study.

FIG. 38 is a Table showing demographic and health characteristics of adults aged 65 and older in the InChianti study who survived or died from all causes or cardiovascular disease during follow-up.

FIG. 39 is a Table showing multivariate cox proportional hazards models examining the relationship between plasma CML and all-cause and CVD mortality.

### DETAILED DESCRIPTION OF THE INVENTION

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

By “Advanced glycation end product (AGE)” is meant a bioactive molecule formed by the non-enzymatic glycation of proteins and other molecules. Methods for measuring CML, including immunoassays, are known in the art and described herein. In one embodiment, CML is measured, for example, using a competitive ELISA (AGE-CML ELISA, Microcoat, Penzberg, Germany)

By “ageing related disease or disorder” is meant a pathology associated with ageing and the accumulation of advanced glycation end products in the body.

By “AGE related disease or disorder” is meant a pathology associated with an accumulation of advanced glycation end products in the body.

As used herein “AGE breaker” or “AGE inhibitor” is understood as one or more of a class of compounds that prevent the formation of advanced glycation endproducts (AGEs) and or the crosslinking of AGEs, or the breaking of cross-linked forms of age. Aminoguanidine was the first drug designed to inhibit glycation reactions by inhibiting the conversion of early products to AGEs. AGE breakers and AGE inhibitors further include, but are not limited to, phenyl-4,5-dimethylthazolium chloride (ALT-711), beta-alanyl-L-histidine, pyridoxamine, carnosine, phenazinediamine, OPB-9195, tenilsetam, phenacylthiazolium, phenacyldimethylthiazolium bromide, and asprin.

By “Receptor for Advanced glycation end product” is meant a circulating or membrane bound polypeptide capable of binding AGEs. RAGEs are described for example by Basta et al., “Receptor for advanced glycation endproducts and atherosclerosis: from basic mechanisms to clinical implications” Atherosclerosis 2008; 196: 9-21,” which is hereby incorporated by reference in its entirety. Circulating isoforms of RAGE include endogenous secretory RAGE (esRAGE), a splice variant of RAGE that is secreted into blood and lacks the transmembrane and cytoplasmic portion of the receptor, and truncated forms of RAGE that have been cleaved from the cell surface by matrix metalloproteinases. Circulating forms of RAGE are described form example by Yonekura et al., “Novel splice variants of the receptor for advanced glycation end-products expressed in human vascular endothelial cells and pericytes, and their putative roles in diabetes-induced vascular injury.” Biochem J 2003; 370: 1097-109.

In one embodiment, a RAGE polypeptide has at least about 85%, 90%, 95% or more sequence identity to NP_{—}001127 or NP_{—}1751947. The sequence of an exemplary RAGE polypeptide is provided below:

```
NCBI Accession No. NP_001127 Advanced glycosylation end product-
specific receptor isoform 1 precursor
    1 maagtavgaw vlvlslwgav vgaqnitari geplvlkckg apkkppqrle wklntgrtea
  61 wkvlspqggg pwdsvarvlp ngslflpavg iqdegifrcq amnrngketk snyrvrvyqi
121 pgkpeivdsa seltagvpnk vgtcvsegsy pagtlswhld gkplvpnekg vsvkeqtrrh
181 petglftlqs elmvtpargg dprptfscsf spglprhral rtapiqprvw epvpleevql
241 vvepeggava pggtvtltce vpaqpspqih wmkdgvplpl ppspvlilpe igpqdqgtys
301 cvathsshgp qesraysisi iepgeegpta gsvggsglgt lalalgilgg lgtaalligv
361 ilwqrrqrrg eerkapenqe eeeeraelnq seepeagess tggp
NCBI Accession No. NP_751947 advanced glycosylation end product-
specific receptor isoform 2 precursor
    1 maagtavgaw vlvlslwgav vgaqnitari geplvlkckg apkkppqrle wklgggpwds
  61 varvlpngsl flpavgiqde gifrcqamnr ngketksnyr vrvyqipgkp eivdsaselt
121 agvpnkvgtc vsegsypagt lswhldgkpl vpnekgvsvk eqtrrhpetg lftlqselmv
181 tparggdprp tfscsfspgl prhralrtap iqprvwepvp leevqlvvep eggavapggt
241 vtltcevpaq pspqihwmkd vsdlergagr trrggancrl cgriragnss pgpgdpgrpg
301 dsrpahwghl vakaatprrg eegprkpggr ggacrtesvg gt
```

Methods for measuring RAGEs are known in the art and described herein. In one embodiment, secretory RAGE is measured using a sandwich ELISA (Quantikine Human RAGE Immunoassay, R&D Systems, Minneapolis, Minn.), which measures C-truncated RAGE that has been enzymatically cleaved from the cell surface as well as endogenous secretory RAGE.

An “agent” is understood herein to include a therapeutically active compound or a potentially therapeutic active compound, e.g., an AGE-breaker or inhibitor. An agent can be a previously known or unknown compound. As used herein, an agent is typically a non-cell based compound, however, an agent can include a biological therapeutic agent, e.g., peptide or nucleic acid therapeutic, cytokine, antibody, etc.

By “alteration” is meant a change (increase or decrease) in the expression levels of a polypeptide as detected by standard art known methods such as those described above. As used herein, an increase or decrease includes a 10% change in expression levels, preferably a 25% change, more preferably a 40% change, and most preferably a 50% or greater change in expression levels. “Alteration” can also indicate a change (increase or decrease) in the biological activity of any of the polypeptides of the invention (e.g., CML or RAGE).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, gamma-carboxyglutamate, and 0-phosphoserine, phosphothreonine.

By “analyte” is meant any compound under investigation using an analytical method.

By “biomarker” is meant any protein, including modified protein, polynucleotide, carbohydrate, or metabolic product having an alteration in expression level or activity that is associated with a disease or disorder, for example an aging related disease or disorder such as, but not limited to, reduced kidney function, renal insufficiency, skeletal muscle strength (grip strength), cardiovascular disease, and anemia.

“Biochip” refers to a solid substrate having a generally planar surface to which an adsorbent is attached. Frequently, the surface of the biochip comprises a plurality of addressable locations, each of which location has the adsorbent bound there. Biochips can be adapted to engage a probe interface, and therefore, function as probes.

“Protein biochip” refers to a biochip adapted for the capture of polypeptides.

As used herein, “changed as compared to a control” sample or subject is understood as having a level of the analyte or diagnostic or therapeutic indicator to be detected at a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an AGE, sRAGE, esRAGE) or a substance produced by a reporter construct (e.g., β-galactosidase or luciferase). Depending on the method used for detection the amount and measurement of the change can vary. Changed as compared to a control reference sample can also include a change in aretherosclerosis, renal function, grip strength, or anemia. Determination of statistical significance is within the ability of those skilled in the art.

“Co-administration” as used herein is understood as administration of one or more agents to a subject such that the agents are present and active in the subject at the same time. Co-administration does not require a preparation of an admixture of the agents or simultaneous administration of the agents.

“Contacting a cell” is understood herein as providing an agent to a test cell e.g., a cell to be treated in culture or in an animal, such that the agent or isolated cell can interact with the test cell or cell to be treated, potentially be taken up by the test cell or cell to be treated, and have an effect on the test cell or cell to be treated. The agent or isolated cell can be delivered to the cell directly (e.g., by addition of the agent to culture medium or by injection into the cell or tissue of interest), or by delivery to the organism by an enteral or parenteral route of administration for delivery to the cell by circulation, lymphatic, or other means.

By “detectable amino acid sequence” or “detectable moiety” is meant a composition that when linked with the nucleic acid or protein molecule of interest renders the latter detectable, via any means, including spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

As used herein, “detecting”, “detection” and the like are understood that an assay performed for identification of a specific analyte in a sample, a product from a reporter construct or heterologous expression construct (e.g., viral vector) in a sample, or an activity of an agent in a sample. Detection can include the determination of the presence and/or quantity of AGE, sRAGE, or esRAGE in a sample. The amount of analyte or activity detected in the sample can be none or below the level of detection of the assay or method.

By “diagnosing” as used herein refers to a clinical or other assessment of the condition of a subject based on observation, testing, or circumstances for identifying a subject having a disease, disorder, or condition based on the presence of at least one sign or symptom of the disease, disorder, or condition. Typically, diagnosing using the method of the invention includes the observation of the subject for more than one sign or symptom of the disease, disorder, or condition.

The phrase “differentially present” refers to differences in the quantity and/or the frequency of a marker present in a sample taken from subjects having human age-related disease as compared to a control subject. For example, serum peptide markers described herein are present at an elevated level in samples of subjects compared to samples from control subjects. In contrast, other markers described herein are present at a decreased level in samples of subjects having or compared to samples from control subjects. Furthermore, a marker can be a polypeptide, which is detected at a higher frequency or at a lower frequency in samples of human subjects at risk for an age-related disease compared to samples of control subjects. A marker can be differentially present in terms of quantity, frequency or both. A polypeptide is differentially present between two samples if the amount of the polypeptide in one sample is statistically significantly different from the amount of the polypeptide in the other sample. Alternatively or additionally, a polypeptide is differentially present between two sets of samples if the frequency of detecting the polypeptide in the subjects' samples is statistically significantly higher or lower than in the control samples.

By “disease associated with elevated serum AGE, sRAGE, and/or esRAGE” or “conditions associated with elevated serum AGE, sRAGE, and/or esRAGE” and the like are understood as one or more disease or condition such as those demonstrated herein to be associated with such levels including, but not limited to renal insufficiency, reduced grip strength, impaired physical performance, increased mortality, particularly increased mortality due to cardiac disease, and anemia, particularly anemia associated with renal disease or anemia of unexplained etiology.

The terms “effective amount,” or “effective dose” refers to that amount of an agent to produce the intended pharmacological, therapeutic or preventive result. The pharmacologically effective amount results in the amelioration of one or more signs or symptoms of a disease or condition or the advancement of a disease or condition, or causes the regression of the disease or condition. For example, a therapeutically effective amount preferably refers to the amount of a therapeutic agent that decreases the level of AGE, sRAGE, or esRAGE in circulation, and/or decreases at least one sign or symptom of a disease or disorder associated with an elevated serum level of AGE, sRAGE, and/or esRAGE. A decrease is preferably a decrease of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, e.g., 2 weeks, one month, 2 months, 3 months, 6 months, one year, 2 years, 5 years, or longer. More than one dose may be required to provide an effective dose.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as a improvement of symptoms, a cure, a reduction in disease signs or symptoms, extension of life, improvement in quality of life, or other effect generally recognized as positive by medical doctors familiar with treating the particular type of disease or condition.

As used herein, “isolated” or “purified” when used in reference to a polypeptide means that a naturally polypeptide or protein has been removed from its normal physiological environment (e.g., protein isolated from plasma or tissue) or is synthesized in a non-natural environment (e.g., artificially synthesized in an in vitro translation system or using chemical synthesis). Thus, an “isolated” or “purified” polypeptide can be in a cell-free solution or placed in a different cellular environment (e.g., expressed in a heterologous cell type). The term “purified” does not imply that the polypeptide is the only polypeptide present, but that it is essentially free (about 90-95%, up to 99-100% pure) of cellular or organismal material naturally associated with it, and thus is distinguished from naturally occurring polypeptide. Similarly, an isolated nucleic acid is removed from its normal physiological environment. “Isolated” when used in reference to a cell means the cell is in culture (i.e., not in an animal), either cell culture or organ culture, of a primary cell or cell line.

As used herein, “kits” are understood to contain at least one non-standard laboratory reagent for use in the methods of the invention. For example, a kit can include an antibody for the specific detection of AGE, sRAGE, or esRAGE and instructions for use, all in appropriate packaging. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

“Obtaining” is understood herein as manufacturing, purchasing, or otherwise coming into possession of.

By “nucleic acid” is meant an oligomer or polymer of at least two ribonucleic acids and/or deoxyribonucleic acids, or analogs thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. For example, pharmaceutically acceptable carriers for administration of cells typically is a carrier acceptable for delivery by injection, and do not include agents such as detergents or other compounds that could damage the cells to be delivered. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations, particularly phosphate buffered saline solutions which are preferred for intraocular delivery.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, intramuscular, intraperotineal, intraocular, intravitreal, subretinal, and/or other routes of parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

As used herein, “plurality” is understood to mean more than one. For example, a plurality refers to at least two, three, four, five, or more.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition, for example an ageing related disease or disorder. Prevention can require the administration of more than one dose of a therapeutic compound.

By “protein”, “peptide” or “polypeptide” is meant any chain of two or more amino acids, or analogs thereof, regardless of length or post-translational modification.

By “reduce or inhibit” is meant the ability to cause an overall decrease preferably of 20% or greater, more preferably of 50% or greater, and most preferably of 75% or greater, in the level of protein.

By “reference” is meant a standard or control condition.

A “sample” as used herein refers to a biological material that is isolated from its environment (e.g., blood or tissue from an animal, cells, or conditioned media from tissue culture) and is suspected of containing, or known to contain an analyte, such as a virus, an antibody, or a product from a reporter construct. A sample can also be a partially purified fraction of a tissue or bodily fluid. A reference sample can be a “normal” sample, from a donor not having the disease or condition fluid, or from a normal tissue in a subject having the disease or condition (e.g., cells from a subject having a mutation that predisposes the subject to RP vs cells from a subject not having a mutation that predisposes the subject to RP). A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested.

As used herein, “serum” refers to the fluid portion of the blood obtained after removal of the fibrin clot and blood cells, distinguished from the plasma in circulating blood. As used herein, “plasma” refers to the fluid, noncellular portion of the blood, distinguished from the serum obtained after coagulation.

As used herein, “sample” or “biological sample” refers to anything, which may contain an analyte (e.g., peptide) for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind including, for example, connective, epithelium, muscle and nerve tissues. Examples of biological tissues also include organs, tumors, lymph nodes, arteries and individual cell(s).

“Solid support” refers to a solid material, which can be derivatized with, or otherwise attached to, a capture reagent. Exemplary solid supports include probes, microtiter plates and chromatographic resins.

The terms “peptide marker”, “marker” and “biomarker” are used interchangeably in the context of the present invention and refer to a polypeptide (of a particular apparent molecular weight), which is differentially present in a sample taken from subjects having human cancer as compared to a comparable sample taken from control subjects (e.g., a person with a negative diagnosis or undetectable cancer, normal or healthy subject). The markers are identified by molecular mass in Daltons, and include the masses centered around the identified molecular masses for each marker.

As used herein, “small molecule” is a molecule, typically an organic molecule, having a molecular weight of no more than 1500 Da, 1000 Da, 750 Da, or 500 Da.

By “specifically binds” is meant a molecule (e.g., peptide, polynucleotide) that recognizes and binds a protein or nucleic acid molecule of the invention, but which does not substantially recognize and bind other molecules in a sample, for example, a biological sample, which naturally includes a protein of the invention.

A “subject” as used herein refers to living organisms. In certain embodiments, the living organism is an animal. In certain preferred embodiments, the subject is a mammal. In certain embodiments, the subject is a domesticated mammal or a primate including a non-human primate. Examples of subjects include humans, monkeys, dogs, cats, mice, rats, cows, horses, goats, and sheep. A human subject may also be referred to as a patient.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with elevated serum levels of AGE, sRAGE, and/or esRAGE are known to those of skill in the art and described in the Examples. Subjects suffering from, and suspected of suffering from, a specific disease, condition, or syndrome are not necessarily two distinct groups.

“Therapeutically effective amount” or “therapeutically effective dose” as used herein refers to an amount of an agent which is effective, upon single or multiple dose administration to the cell or subject, in prolonging the survivability of the patient with such a disorder, reducing one or more signs or symptoms of the disorder, preventing or delaying and the like beyond that expected in the absence of such treatment.

An agent or other therapeutic intervention can be administered to a subject, either alone or in combination with one or more additional therapeutic agents or interventions, as a pharmaceutical composition in mixture with conventional excipient, e.g., pharmaceutically acceptable carrier, or therapeutic treatments.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, Pa., 1985). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain agents.

It will be appreciated that the actual preferred amounts of active compounds used in a given therapy will vary according to e.g., the specific compound being utilized, the particular composition formulated, the mode of administration and characteristics of the subject, e.g., the species, sex, weight, general health and age of the subject. Optimal administration rates for a given protocol of administration can be readily ascertained by those skilled in the art using conventional dosage determination tests conducted with regard to the foregoing guidelines.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more as compared to subjects in an appropriate age, gender, etc matched control group.

As used herein, the terms “treat,” “treating,” “treatment,” and the like refer to reducing or ameliorating a disorder and/or at least one sign or symptom associated therewith. It will be appreciated that, although not precluded, treating a disorder or condition does not require that the disorder, condition or the signs or symptoms associated therewith be completely eliminated. Treatment can require the administration of more than one dose of a therapeutic compound.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other definitions appear in context throughout the disclosure.

### Methods of the Invention

The invention generally features serum carboxymethyl-lysine (CML) and circulating receptor for advanced glycation product (RAGE) that can be used as clinical biomarkers or as a test to identify people who are at an increased risk of dying from cardiovascular disease, developing chronic kidney disease, developing loss of muscle strength, and anemia.

Advanced glycation products (AGEs) are bioactive molecules that are formed by the non-enzymatic glycation of proteins, lipids, and nucleic acids. AGEs have been implicated in the pathogenesis of diabetes, atherosclerosis, and renal disease. AGEs induce covalent cross-links with proteins such as collagen, contribute to arteriosclerosis and atherosclerosis, and increased glomerular sclerosis and interstitial fibrosis in the kidney. AGEs upregulate inflammation through RAGE. CML is a dominant AGE that is found in serum and tissues. Serum carboxymethyl-lysine and serum RAGE can be measured and used as a clinical test to identify people who are at higher risk of cardiovascular disease mortality, chronic kidney disease, loss of muscle strength, and anemia. Serum carboxymethyl-lysine is an important and useful clinical test because serum carboxymethyl-lysine levels can be lowered by modifying dietary intake of AGEs and by pharmacological intervention.

### Methods and Peptide Profiles of the Invention

The present invention provides peptide markers (e.g., CML and/or RAGE) generated from comparisons of protein profiles from subjects diagnosed with age-related disease (e.g., having, or having a propensity to develop reduced kidney function, renal insufficiency, skeletal muscle strength, sarcopenia, impaired physical performance, cardiovascular disease, cardiovascular disease-related death, anemia) and from subjects without known age-related disease diseases. In particular, the invention provides that these markers, used individually or in combination with other markers, provide a method of diagnosing and monitoring age-related disease.

Markers that are differentially present in samples of subjects at risk for an age-related disease and control subjects find application in methods and kits for determining age-related disease status. Accordingly, methods are provided for identifying age-related disease in a subject comprising detecting a differential presence of a biomarker in subjects having an age-related disease vs. subjects without such diseases. The amount of one or more biomarkers found in a test sample compared to a control, or the presence or absence of one or more markers in the test sample provides useful information regarding the disease status of the patient.

A. Types of Samples

The markers (e.g., CML, AGEs, RAGE) can be measured in different types of biological samples. The sample is preferably a biological fluid sample. Examples of a biological samples useful in this invention include blood, blood serum, plasma, urine, tears, saliva, tissue, cells. Blood serum is a preferred sample source for embodiments of the invention.

If desired, the sample can be prepared to enhance detectability of the markers. For example, to increase the detectability of markers, a blood serum sample from the subject can be preferably fractionated. The method of fractionation depends on the type of detection method used. Any method that enriches for the protein of interest can be used. Typically, preparation involves fractionation of the sample and collection of fractions determined to contain the biomarkers. Methods of pre-fractionation include, for example, size exclusion chromatography, ion exchange chromatography, heparin chromatography, affinity chromatography, sequential extraction, gel electrophoresis and liquid chromatography. The analytes also may be modified prior to detection. These methods are useful to simplify the sample for further analysis. For example, it can be useful to remove high abundance proteins, such as albumin, from blood before analysis.

B. Detection of Serum Peptide Markers

As reported herein, levels of CML and/or RAGE correlate risk for an age-related disease. In one approach, subjects at risk for an age-related disease are identified by measuring CML and/or RAGE in a biological sample. In one embodiment, blood serum from the subject is measured for levels of CML or RAGE. Methods used to measure serum levels of proteins include ELISA, western blotting, or immunoassays using specific antibodies. In one embodiment, CML and/or RAGE is detected using a moiety that binds the analyte fixed to a solid support. The physical shape of the solid support is not critical, although some shapes may be more convenient than others for the present purpose. Accordingly, the solid support may be in the shape of a paper strip, dipstick, membrane (e.g. a nylon membrane or a cellulose filter), a plate (e.g. a microtiter plate) or solid particles (e.g. latex beads). The solid support may be made of any suitable material, including but not limited to a plastic (e.g., polyethylene, polypropylene, polystyrene, latex, polyvinylchloride, polyurethane, polyacrylamide, polyvinylalcohol, nylon, polyvinyl acetate, or any suitable copolymers thereof), cellulose (e.g. various types of paper, such as nitrocellulose paper and the like), a silicon polymer (e.g. siloxane), a polysaccharide (e.g. agarose or dextran), or an ion exchange resin (e.g. conventional anion or cation exchange resins).

For some of the method embodiments of the invention, it may be helpful to purify the marker detected by the methods disclosed herein prior to subsequent analysis. Nearly any means known to the art for the purification and separation of small molecular weight substances, e.g., anion or cation exchange chromatography, gas chromatography, liquid chromatography or high pressure liquid chromatography may be used. Methods of selecting suitable separation and purification techniques and means of carrying them out are known in the art (see, e.g., Labadarious et. al., J. Chromatography (1984) 310:223-231, and references cited therein; and Shahrokhin and Gehrke, J. Chromatography (1968) 36:31-41, and Niessen J. Chromatography (1998) 794:407-435).

Methods of Detection

Any suitable method can be used to detect one or more of the markers described herein. Successful practice of the invention can be achieved with one or a combination of methods that can detect and, preferably, quantify the markers. These methods include, without limitation, hybridization-based methods including those employed in biochip arrays, mass spectrometry (e.g., laser desorption/ionization mass spectrometry), fluorescence (e.g. sandwich immunoassay), surface plasmon resonance, ellipsometry and atomic force microscopy. Methods may further include, by one or more of electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)n, atmospheric pressure photoionization mass spectrometry (APPI-MS), APPI-MS/MS, and APPI-(MS)n, quadrupole mass spectrometry, fourier transform mass spectrometry (FTMS), and ion trap mass spectrometry, where n is an integer greater than zero.

Biochip-Based Methods

Detection methods may include use of a biochip array. Biochip arrays useful in the invention include protein arrays. One or more markers are captured on the biochip array and subjected to laser ionization to detect the molecular weight of the markers. Analysis of the markers is, for example, by molecular weight of the one or more markers against a threshold intensity that is normalized against total ion current.

The biochip surfaces may, for example, be ionic, anionic, hydrophobic; comprised of immobilized nickel or copper ions, comprised of a mixture of positive and negative ions; and/or comprised of one or more antibodies, single or double stranded nucleic acids, proteins, peptides or fragments thereof, amino acid probes, or phage display libraries. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International publication WO 00/56934 (Englert et al., “Continuous porous matrix arrays,” Sep. 28, 2000).

Markers may be captured with capture reagents immobilized to a solid support, such as a biochip, a multiwell microtiter plate, a resin, or nitrocellulose membranes that are subsequently probed for the presence of proteins. Capture can be on a chromatographic surface or a biospecific surface. For example, a sample containing the markers, such as serum, may be placed on the active surface of a biochip for a sufficient time to allow binding. Then, unbound molecules are washed from the surface using a suitable eluant, such as phosphate buffered saline. In general, the more stringent the eluant, the more tightly the proteins must be bound to be retained after the wash.

Upon capture on a biochip, analytes can be detected by a variety of detection methods selected from, for example, a gas phase ion spectrometry method, an optical method, an electrochemical method, atomic force microscopy and a radio frequency method. Gas phase ion spectrometry methods are described herein. Of particular interest is the use of mass spectrometry, and in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

Mass Spectrometry-Based Methods

Mass spectrometry (MS) is a well-known tool for analyzing chemical compounds. Thus, in one embodiment, the methods of the present invention comprise performing quantitative MS to measure the serum peptide marker. The method may be performed in an automated (Villanueva, et al., Nature Protocols (2006) 1(2):880-891) or semi-automated format. This can be accomplished, for example with MS operably linked to a liquid chromatography device (LC-MS/MS or LC-MS) or gas chromatography device (GC-MS or GC-MS/MS). Methods for performing MS are known in the field and have been disclosed, for example, in US Patent Application Publication Nos: 20050023454; 20050035286; U.S. Pat. No. 5,800,979 and references disclosed therein.

The protein fragments, whether they are peptides derived from the main chain of the protein or are residues of a side-chain, are collected on the collection layer. They may then be analyzed by a spectroscopic method based on matrix-assisted laser desorption/ionization (MALDI) or electrospray ionization (ESI). The preferred procedure is MALDI with time of flight (TOF) analysis, known as MALDI-TOF MS. This involves forming a matrix on the membrane, e.g. as described in the literature, with an agent which absorbs the incident light strongly at the particular wavelength employed. The sample is excited by UV, or IR laser light into the vapour phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are commercially available from PerSeptive Biosystems, Inc. (Frazingham, Mass., USA) and are described in the literature, e.g. M. Kussmann and P. Roepstorff, cited above.

Magnetic-based serum processing can be combined with traditional MALDI-TOF. Through this approach, improved peptide capture is achieved prior to matrix mixture and deposition of the sample on MALDI target plates. Accordingly, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the invention to produce an array of spots on the collecting membrane, and the array may be analyzed. Subsequently, automated output of the results is provided by using the ExPASy server, as at present used for MIDI-TOF MS and to generate the data in a form suitable for computers.

In an additional embodiment of the methods of the present invention, multiple markers are measured. The use of multiple markers increases the predictive value of the test and provides greater utility in diagnosis, toxicology, patient stratification and patient monitoring. The process called “Pattern recognition” detects the patterns formed by multiple markers greatly improves the sensitivity and specificity of clinical proteomics for predictive medicine. Subtle variations in data from clinical samples indicate that certain patterns of protein expression can predict phenotypes such as the presence or absence of a certain disease, or a positive or adverse response to drug treatments.

C. Data Analysis

Data generated by detection of markers (e.g., CML, AGEs, RAGE) can be analyzed using any suitable means. In one embodiment, data is analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. The computer also contains code that receives as input, data on the strength of the signal at various molecular masses received from a particular addressable location on the probe. This data can indicate the number of markers detected, including the strength of the signal generated by each marker.

Data analysis can include the steps of determining signal strength (e.g., height of peaks) of a marker detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each marker or other biomolecules can be displayed in the form of relative intensities in the scale desired (e.g., 100). Alternatively, a standard (e.g., a serum protein) may be admitted with the sample so that a peak from the standard can be used as a reference to calculate relative intensities of the signals observed for each marker or other markers detected.

The computer can transform the resulting data into various formats for displaying. For each sample, markers that are detected and the amount of markers present in the sample can be saved in a computer readable medium. This data can then be compared to a control (e.g., a profile or quantity of markers detected in control, e.g., subjects in whom human disease is undetectable).

When the sample is measured and data is generated the data is then analyzed by a computer software program. Generally, the software can comprise code that converts signal from the detection module into computer readable form. The software also can include code that applies an algorithm to the analysis of the signal to determine whether the signal represents a “peak” in the signal corresponding to a marker of this invention, or other useful markers. The software also can include code that executes an algorithm that compares signal from a test sample to a typical signal characteristic of “normal” and human age-related disease (e.g., reduced kidney function, renal insufficiency, skeletal muscle strength, sarcopenia, cardiovascular disease, cardiovascular disease-related death, and anemia) and determines the closeness of fit between the two signals. The software also can include code indicating which the test sample is closest to, thereby providing a probable diagnosis.

### Microarrays

CML and RAGEs can be fixed to a substrate and analyzed alone or in combination with one or more additional biomarkers. Such biomarkers may conveniently be analyzed, for example, using microarrays. Typically, microarrays feature a biomarker, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., biomarkder or antibodies against such biomarkers) are spotted on a substrate using any convenient method known to the skilled artisan (e.g., by hand or by inkjet printer). Preferably, such methods retain the biological activity or function of the protein bound to the substrate. Methods for making such arrays are known in the art and described for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

A biomarker microarray is hybridized with a detectable probe. Such probes can be polypeptide (e.g., antibodies), nucleic acid, or small molecules. For some applications, polypeptide and nucleic acid probes are derived from a biological sample taken from a patient, such as a bodily fluid (such as blood, urine, saliva, or phlegm); a homogenized tissue sample (e.g. a tissue sample obtained by biopsy); or cultured cells (e.g., lymphocytes). Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. A fter removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

### Biomarker Combinations

Biomarker combinations useful in the invention include any polypeptide indicative of an AGE-related disease or disorder (e.g., reduced kidney function, renal insufficiency, skeletal muscle strength, sarcopenia, impaired physical performance, cardiovascular disease, cardiovascular disease-related death, and anemia). For example, cardiovascular biomarkers useful in combination with CML or RAGE include, but are not limited to, high density lipoprotein, low density lipoprotein, C reactive protein, total cholesterol, and triglycerides. Methods for detecting such biomarkers are known in the art and are described herein. CML, RAGE, and the aforementioned biomarkers may be analysed using a microarray, may be analysed in combination during a blood test, or may be analysed in a kit of the invention.

### Kits

The invention provides kits for the diagnosis of an age-related disease or disorder. In one embodiment, the kit includes a composition (e.g., antibody) that detects CML, RAGE, or another biomarker of the invention. In some embodiments, the kit comprises a sterile container which contains a diagnostic composition; such containers can be boxes, ampoules, bottles, vials, tubes, bags, pouches, blister-packs, or other suitable container forms known in the art. In other embodiments, the kit comprises a substrate (e.g., plate or other container comprising wells) suitable for use in an ELISA for detecting CML or RAGE. Such containers can be made of plastic, glass, laminated paper, metal foil, or other materials suitable for holding medicaments.

If desired a diagnostic kit of the invention is provided together with instructions for detecting a CML or RAGE in a subject having or at risk of developing an age-related disease or disorder (e.g., (e.g., reduced kidney function, renal insufficiency, skeletal muscle strength, sarcopenia, impaired physical performance, cardiovascular disease, cardiovascular disease-related death, and anemia), and for assessing the risk of the disease in the subject. The instructions will generally include information about the use of the composition for the diagnosis of an age-related disease or a propensity to develop such a disease or disorder. The instructions may be printed directly on the container (when present), or as a label applied to the container, or as a separate sheet, pamphlet, card, or folder supplied in or with the container.

### Selection of a Treatment Method

After a subject is diagnosed as having or having a propensity to develop an age-related disease or disorder a method of treatment is selected. Where a subject is identified as having an increased level of CML and/or RAGEs relative to a reference (e.g., a 5% 10%, 20%, 30%, 40%, 50%, 60%, 70%, 80%, 90%), which correlates with a propensity to develop an age-related disease, the subject may be treated by imposing certain dietary restrictions. For example, the subjects intake of AGE-containing foods may be reduced. AGE-containing foods include foods processed at high temperatures, deep fried, oven fried, grilled, or broiled. Subsequent to the dietary restrictions, the subject's CML levels are measured. Failure to adequately reduce AGE levels to control levels identifies the subject as in need of more aggressive treatment, such as treatment with AGE inhibitors or AGE breakers.

### Patient Monitoring

The diagnostic methods of the invention are useful for monitoring CML and/or RAGE in a patient or for assessing the efficacy of a therapeutic regimen (e.g., pharmacological intervention or dietary restrictions). In one embodiment, the diagnostic methods of the invention are used periodically to monitor the CML and/or RAGE levels. In one example, the subject's CML and/or RAGE levels is characterized using a diagnostic assay of the invention prior to administering therapy. A subject's CML and/or RAGE levels may be considered alone or in combination with other physical measures of health. This assay provides a baseline that describes the level of one or more biomarkers prior to treatment. Additional diagnostic assays are administered during the course of therapy to monitor the efficacy of a selected therapeutic regimen. A therapy is identified as efficacious when a diagnostic assay of the invention detects a reduction in CML or RAGE levels relative to the baseline levels of these biomarker.

### Therapeutics

Subjects identified as at risk for an age-related disease (e.g., reduced kidney function, renal insufficiency, skeletal muscle strength, sarcopenia, impaired physical performance, cardiovascular disease, cardiovascular disease-related death, and anemia) may be treated with compounds described herein, including AGE-breakers and/or AGE inhibitors. The compounds of the invention can, for example, be administered by injection, for example intravenously, intraarterially, subdermally, intraperitoneally, intramuscularly, or subcutaneously; or orally, buccally, nasally, transmucosally, or topically with a dosage ranging from about 0.001 to about 100 mg/kg of body weight, or according to the requirements of the particular drug and more preferably from 0.5-10 mg/kg of body weight.

Frequency of dosing will depend on the agent admistered, the progression of the disease or condition in the subject, and other considerations known to those of skill in the art. For example, dosing can be performed 1, 2, 3, 4 or more times daily; 1, 2, 3, 4, or more times weekly, 1, 2, 3, 4, or more times monthly, every other month, every three months, every four months, every six months, annually, or at any other regular or irregular dosing intervals. Dosing may be determined in conjunction with monitoring of one or more signs or symptoms of the specific disease or diseases that the subject is suffering from or suspected of suffering from.

The amount of active ingredient that may be combined with the carrier materials to produce a single dosage form will vary depending upon the host treated and the particular mode of administration. A typical preparation will contain from about 1% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

The term “pharmaceutically acceptable carrier” refers to a carrier that can be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α.-tocopherol polyethyleneglycol 1000 succinate, surfactants used in pharmaceutical dosage forms such as Tween® or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, may also be advantageously used to enhance delivery of compounds of the formulae described herein.

The pharmaceutical compositions of this invention may be administered enterally for example by oral administration, parenterally, intraocularly, by inhalation spray, topically, nasally, buccally, or via an implanted reservoir, preferably by oral or vaginal administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases, or buffers to enhance the stability of the formulated compound or its delivery form. The term parenteral as used herein includes intraocular, subcutaneous, intracutaneous, intravenous, intramuscular, intraarticular, intraarterial, intrasynovial, intrastemal, intrathecal, intralesional, and intracranial injection or infusion techniques.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, TWEEN® 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as TWEENs® or SPANs® and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of the invention may be administered topically. The pharmaceutical composition will be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

Effective dosages of the compounds of the invention to be administered may be determined through procedures well known to those in the art that address such parameters as biological half-life, bioavailability, and toxicity.

### EXAMPLES

### Example 1

### Advanced Glycation End Products and their Circulating Receptors and Level of Kidney Function

Advanced glycation end products (AGEs) and the receptor for AGE (RAGE) are implicated in the pathogenesis of renal disease but their relation with level of kidney function has not been well characterized.

Chronic kidney disease affects more than fifteen million people in the United States and is associated with high cardiovascular disease morbidity and mortality. The factors that affect the progression of chronic kidney disease have been incompletely characterized. Advanced glycation end products (AGES) are bioactive molecules implicated in the pathogenesis of chronic kidney disease, diabetes, and atherosclerosis. AGEs are formed by the non-enzymatic glycation of proteins and other molecules. Two major sources of AGEs are exogenous AGEs ingested in foods and endogenous AGEs formed in the body. AGEs accumulate in tissues, and the rate accelerates with aging. The western diet is rich in AGEs, as AGEs are formed when food is processed at elevated temperatures, i.e., deep frying, broiling, and grilling. About 10% of ingested AGEs are absorbed and two-thirds are retained in tissues. In humans, lower dietary intake of AGEs reduces serum AGEs, decreases inflammation, and improves vascular function.

AGE-breakers or inhibitors improve arterial compliance, cardiac function, and renal function in humans and animal models.

AGEs upregulate inflammation through the receptor for AGEs (RAGE) (Basta G, Atherosclerosis 196: 9-21, 2008; Schmidt et al, J Biol Chem 267:14987-14997, 1992; Neeper et al, J Biol Chem 267:14998-15004, 1992; Yonekura et al, Biochem J 370:1097-1109, 2003.) Circulating isoforms of RAGE include endogenous secretory RAGE (esRAGE), a splice variant of RAGE that is secreted into blood and lacks the transmembrane and cytoplasmic portion of the receptor and truncated forms of RAGE that have been cleaved from the cell surface by matrix metalloproteinases. The relation between sRAGE and esRAGE with chronic kidney disease has not been well characterized, and there may be differences between concentrations of the two circulating receptors because esRAGE is expressed after transcriptional activation. Circulating RAGE can bind AGE and prevent AGE activation of cell membrane-bound RAGE (Wautier et al, J Clin Invest 97:238-243, 1996). Circulating RAGE may serve as a decoy receptor to counteract the inflammatory processes triggered by RAGE ligands such as AGEs (Geroldi et al., Curr Med Chem 13:1971-1978, 2006).

The AGE-RAGE pathway has been the focus of growing interest because of substantial improvement in measurement technology and because experiments conducted in animal models have shown that blockage of AGE-RAGE binding reduces complications of atherosclerosis and diabetes (Basta G, Atherosclerosis 196: 9-21, 2008). Total circulating RAGE (sRAGE) and esRAGE have been studied in specific groups of patients with diabetes (Challier et al., Clin Chem 51:1749-1750, 2005; Tan et al., Diabetologia 49:2756-2762, 2006) and end-stage renal disease (Sakurai et al. Diabetes Res Clin Pract 73:158-165, 2006; Kalousovd et al., Am J Kidney Dis 47:406-411, 2006). It was postulated that elevated levels of serum AGE, sRAGE, and esRAGE were associated with reduced level of kidney function and were predictive of reduced level of kidney function in subjects with normal baseline renal function. To address this hypothesis, AGE, sRAGE, and esRAGE were characterized in a prospective study of older women living in the community.

Study Population

Subjects in this study were women, aged 65 and older, who participated in the Women's Health and Aging Study I (WHAS I), a population-based study designed to evaluate the causes and course of physical disability in older disabled women living in the community. WHAS I participants were recruited from an age-stratified random sample of women aged 65 years and older selected from Medicare enrollees residing in 12 contiguous zip code areas in Baltimore. Women were screened to identify self-reported physical disability that was categorized into four domains. The domains of disability were ascertained in a 20-30 minute home interview that included questions related to (1) mobility and exercise tolerance, i.e., walking for a quarter of a mile, walking up 10 steps without resting, getting in and out of bed or chairs, (2) upper extremity function, i.e., raising your arms up over your head, using your fingers to grasp or handle, lifting or carrying something as heavy as ten pounds, (3) higher functioning tasks (a subset of instrumental activities of daily living, not including heavy housework, i.e., using the telephone, doing light housework, preparing your own meals, shopping for personal items), and (4) basic self-care tasks (a subset of non-mobility dependent activities of daily living, i.e., bathing or showering, dressing, eating, using the toilet). WHAS I enrolled the one-third most disabled women ages 65 and older, those with disability in two or more domains. Of the 1409 women who met study eligibility criteria, 1002 agreed to participate in the study in 1992. There were no major differences in sociodemographic or reported health characteristics between eligible participants and those who declined to participate (Guralnik et al., The Women's Health and Aging Study: Health and Social Characteristics of Older Women with Disability. Bethesda, Md., National Institute on Aging. NIH Publication No. 95-4009, 1995, incorporated herein by reference).

Data Collection

Standardized questionnaires were administered in the participant's home by trained interviewers. Race was assessed in a questionnaire as African-American, white, or other, current smoking as yes or no, and education as 0-8, 9-11, 12 years or more than 12 years as the highest level of formal education achieved. Two weeks later, a trained registered full-time study nurse practitioner examined each study participant in her home, using a standardized evaluation of physical performance and physical exam. Approximately 75% of women also consented to phlebotomy performed during a separate visit by a trained phlebotomist who followed a standardized protocol. The definitions for the chronic diseases reported in this study were adjudicated by WHAS co-investigators based on standardized algorithms that combined information from the questionnaire, physical examination, and physician contact (Guralnik et al., 1995). The Mini-Mental Status Examination (MMSE) was administered at enrollment (Folstein et al., J Psychiatr Res 12:189-198, 1975). Women were seen every 6 months for a follow-up visit for 36 months, and phlebotomy was repeated at the 12 and 24 month follow-up visits. Further details on the methods and sampling design of the WHAS studies are published elsewhere (Guralnik et al., 1995). The study protocol was adherent to the Declaration of Helsinki. The Johns Hopkins University Institutional Review Board approved the study protocol, and written informed consent was obtained from all participants.

Laboratory Studies

There were 1002 women enrolled in the Women's Health and Aging Study I. At the 12-month follow-up visit, 879 women returned for follow-up, of which 580 received a blood draw. AGE and RAGE were measured in 548 women who had serum creatinine measurements available. The 548 women involved in the present study were significantly younger, and a higher proportion had MMSE score <23, level of education <12 years, and stroke compared with the 331 women who are not included in the present analysis. Laboratory measurements of serum AGEs, sRAGE, and esRAGE were done at the 12-month follow-up visit rather than at enrollment because of a greater availability of serum aliquots in the sample repository from this visit. Thus, we will refer to 12-month follow-up visit as the baseline visit for this study. Non-fasting blood samples were obtained by venipuncture between 9 AM and 2 PM. Serum creatinine was measured at Quest Diagnostics Laboratories (formerly Ciba-Corning Laboratories, Baltimore, Md.) using the Jaffe method. Processing, aliquoting, and freezing were carried out at the Core Genetics Laboratory of the Johns Hopkins University School of Medicine following a standardized protocol. Blood samples were stored continuously at −70° C. until the time of analyses of serum AGEs, sRAGE, and esRAGE.

The measure of serum AGEs in this study was serum carboxymethyl-lysine (CML). CML is a dominant circulating AGE, the best characterized of all the AGEs, and a dominant AGE in tissue proteins (Reddy et al., Biochemistry 34:10872-10878, 1995). Total CML was measured using a competitive ELISA (AGE-CML ELISA, Microcoat, Penzberg, Germany) (Boehm et al., Diabetologia 2004; 47: 1376-1379, incoroporated herein by reference). This assay has been validated (Zhang et al., Clin Chem Lab Med 2005; 43: 503-511, incorporated herein by reference), is specific, and shows no cross-reactivity with other compounds. Total sRAGE was measured using a sandwich ELISA (Quantikine Human RAGE Immunoassay, R & D Systems, Minneapolis, Minn.). This assay measures C-truncated RAGE that has been enzymatically cleaved from the cell surface as well as esRAGE. Serum esRAGE was measured using ELISA (B-Bridge International, Mountain View, Calif.) (Sakurai et al., Diabetes Res Clin Pract 73:158-165, 2006). Measurements were all performed in duplicate according to the protocol of the manufacturers, and the results were averaged. The within assay and between assay coefficients of variation (CVs) for serum CML, sRAGE, and esRAGE were 3% and 4%, 3% and 7%, and 6% and 8%, respectively. The Spearman correlations between CML, and sRAGE, and esRAGE, respectively, were r=0.18 and r=0.18 (both P<0.001), and between sRAGE and esRAGE, was r=0.89 (P<0.001).

Statistical Analysis

Continuous variables were compared using Wilcoxon rank-sum test.

Categorical variables were compared using chi-square tests. Body mass index (BMI) was categorized as underweight (<18.5 kg/m²), normal range (18.5-24.9 kg/m²), overweight (25-29.9 kg/m²) and obese (>30 kg/m²). A Mini-Mental Status Examination score of <23 was defined as cognitive impairment. Reduced glomerular filtration rate (GFR) was defined as estimated GFR of <60 mL/min/1.73 m² using the 4-variable Modification of Diet in Renal Disease (MDRD) Study equation of Levey and colleagues (Levey et al., Ann Intern Med 130:461-470, 1999). Logistic regression models were used to examine separately the relationships of serum CML, sRAGE, and esRAGE, with prevalent reduced GFR at baseline and prevalent reduced GFR 12 months later, excluding prevalent cases of reduced GFR at baseline. Linear regression models were used to examine the same cross-sectional relationships where the dependent variable was estimated GFR at baseline. Variables that were significant in the univariate analyses were entered into the multivariate logistic regression models and multivariate linear regression models. Diabetes was added in alternative multivariate models because of the known strong relationship between diabetes and chronic kidney disease. In linear and logistic regression models, a one standard deviation in concentration of serum CML, sRAGE, and esRAGE, respectively, was used as the unit of change. Spearman correlation was used for examining correlation between serum CML, esRAGE, and sRAGE. The statistical program used was SAS (SAS Institute, Cary, N.C.), with data analysis conducted by Kai Sun.

The level of significance used in this study was P<0.05.

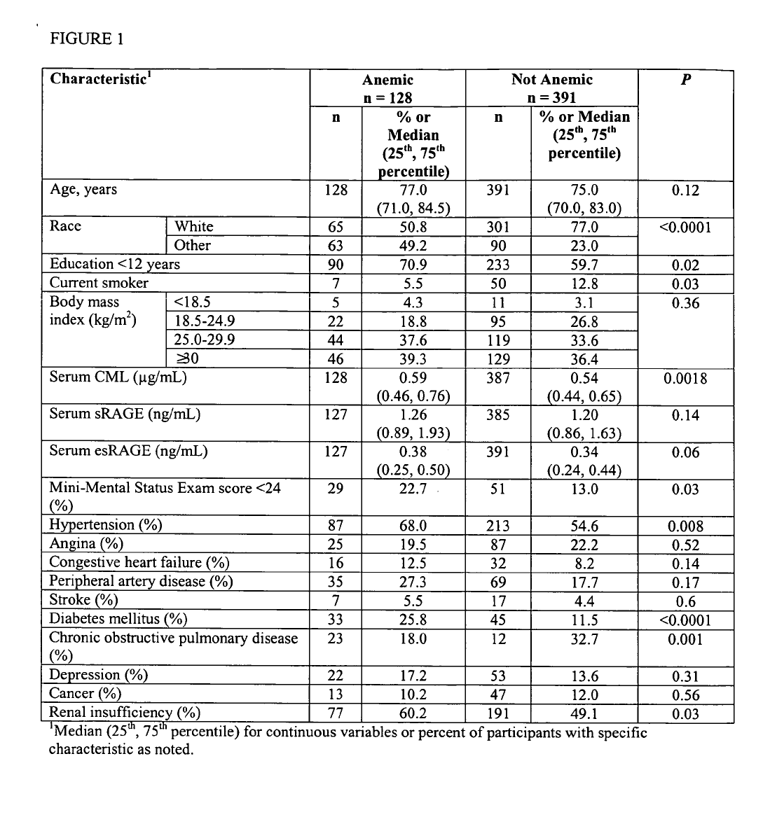

The demographic and health characteristics of 548 women with and without reduced GFR are shown in the Table in FIG. 1.

Overall, mean (SD) serum creatinine at baseline was 1.1 (0.3) mg/dL, or 97 (27) μmol/L, and mean (SD) estimated GFR was 60.1 (16.2) mL/min/1.73 m². Women with reduced GFR were more likely to be older, non-white, and to have coronary artery disease, congestive heart failure, and peripheral artery disease. There were no significant differences in education, current smoking, body mass index, cognitive function, or prevalence of hypertension, diabetes, stroke, chronic obstructive pulmonary disease, depression, or cancer between women with and without reduced GFR. Median serum CML, sRAGE, and esRAGE concentrations were significantly higher in women with reduced GFR compared with women without reduced GFR.

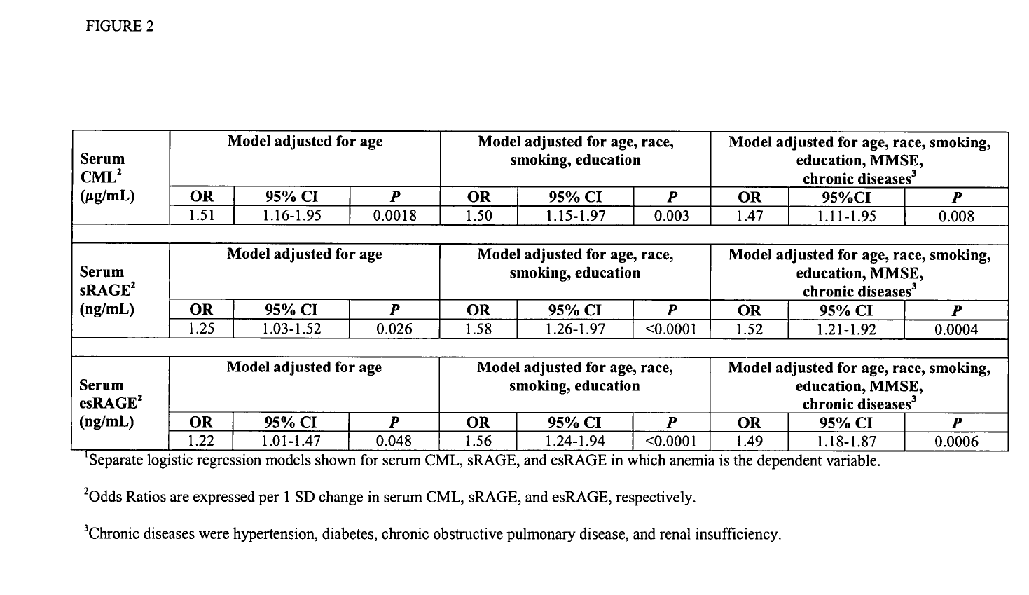

Separate multivariate logistic regression models were used first to examine the cross-sectional relationship between serum CML, sRAGE, and esRAGE with reduced GFR (as shown in the Table in FIG. 2).

In models adjusted for age, and adjusted additional for race, hemoglobin Alc, and coronary heart disease, congestive heart failure, and peripheral artery disease, serum CML, sRAGE, and esRAGE were all significantly associated with increased odds of prevalent reduced GFR, as shown in the Table in FIG. 2. Diabetes was not significantly associated with prevalent reduced GFR in the univariate analyses, but alternative models were run in which diabetes was added to a multivariate model as in Table 2 that included age, race, hemoglobin Alc, and chronic diseases. Serum CML, sRAGE, and esRAGE (per 1 Standard Deviation [S.D.] increase) were associated with reduced GFR when diabetes was added to the respective multivariate models: O.R. 1.98, 95% C.I. 1.42-2.77, P<0.001; O.R. 1.42, 95% C.I. 1.14-1.76, P=0.002; O.R. 1.41, 95% C.I. 1.12-1.78, P<0.001.

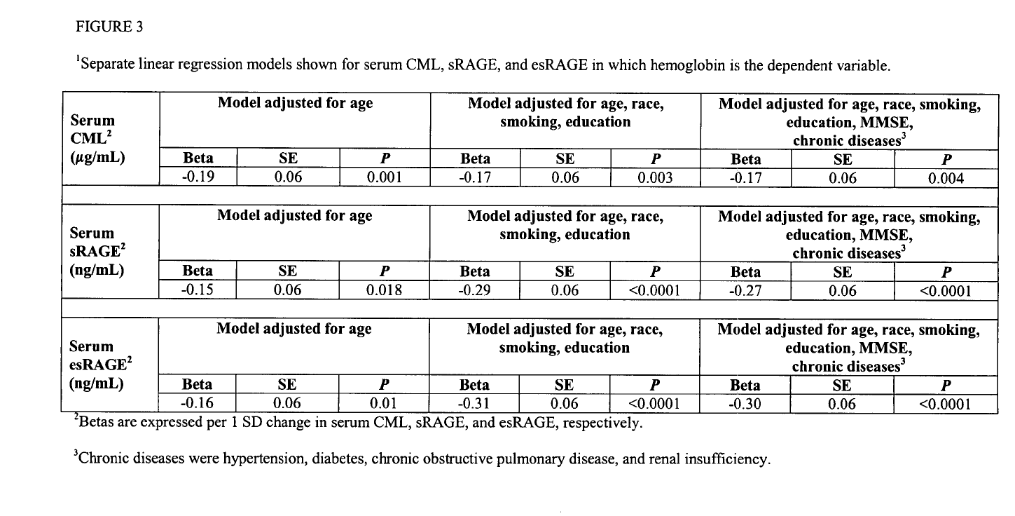

Serum CML, sRAGE, and esRAGE (per 1 S.D. increase), respectively, were associated with estimated GFR at baseline in separate linear regression models adjusting for age, and additionally adjusting for race, hemoglobin Alc coronary heart disease, congestive heart failure, and peripheral artery disease (see Table shown in FIG. 3). Alternative models for serum CML, sRAGE, and esRAGE (per 1 S.D. increase), respectively, were also considered in which diabetes was added to the model, in addition to age, race, hemoglobin A1, coronary heart disease, congestive heart failure, and peripheral artery disease: beta=−4.10, SE=0.68, P<0.001; beta=−3.84, SE=0.73, P<0.001; beta=−3.25, SE=0.74, P<0.001, respectively.

Of the 548 women seen at baseline, 376 women were seen in follow-up 12 months later. Of 230 women without reduced GFR at baseline, 32 (13.9%) women developed reduced GFR by the follow-up visit 12 months later. Serum CML (μg/mL) at baseline, per 1 S.D. increase, was associated with prevalence of reduced GFR at 12 months (O.R. 1.80, 95% C.I. 1.19-2.71, P=0.005) in a multivariate logistic regression model adjusting for age, race, hemoglobin A1, coronary heart disease, congestive heart failure, and peripheral artery disease. Adding diabetes to the previous model yielded similar results (O.R. 1.80, 95% C.I. 1.19-2.71, P=0.005). Serum sRAGE (ng/mL) at baseline, per 1 S.D. increase, was associated with prevalence of reduced GFR at 12 months (O.R. 1.32, 95% C.I. 1.01-1.74, P=0.05). Adding diabetes to the previous model yielded similar results (O.R. 1.32, 95% C.I. 1.01-1.74, P=0.04). Serum esRAGE (ng/mL) at baseline, per 1 S.D. increase, was associated with prevalence of reduced GFR at 12 months (O.R. 1.33, 95% C.I. 1.01-1.77, P=0.05) in a multivariate logistic regression model adjusting for age, race, coronary heart disease, congestive heart failure, and peripheral artery disease. Adding diabetes to the previous model yielded similar results (O.R. 1.33, 95% C.I. 1.01-1.77, P=0.04).

At baseline, among 82 women with diabetes and 466 women without diabetes, mean (SD) serum CML, sRAGE, and esRAGE concentrations were, respectively, 0.55 (0.2) and 0.61 itg/mL (P=0.08), 1.35 (0.79) and 1.35 (0.70) ng/mL (P=0.9), and 0.37 (0.24) and 0.38 (0.21) ng/mL (P=0.7).

The results presented herein demonstrate that elevated serum CML and circulating RAGE are associated with reduced GFR in older community-dwelling women and suggests that these associations are independent of the multiple morbidities present in this high-risk, disabled population. Elevated circulating AGEs have been described in diabetes and in chronic kidney disease with or without diabetes. Patients with chronic kidney disease and end-stage renal disease were found to have elevated RAGE expression and circulating RAGE, respectively. RAGE mRNA is increased in peripheral mononuclear cells obtained from patients with chronic kidney disease. Increased levels of RAGE may be a protective mechanism against the pro-inflammatory effect of circulating AGE on cells. The present study shows that elevated serum AGEs and circulating RAGE are associated with reduced GFR in a population-based study of community-dwelling adults. The present study also suggests that elevated serum AGEs and circulating RAGE are predictive of the development of reduced GFR, but some caution must be taken in the interpretation of these findings, since the number of cases was relatively small and the follow-up limited to only one year.

AGEs are metabolized and removed by the kidney but the kidney is also a site for accumulation of AGEs and AGE-related damage (Gugliucci and Bendayan, Diabetologia 39:149-160, 1996; Miyata et al., Kidney Int 53:416-422, 1998; Schinzel et al., Nephron 87:295-303, 2001). The serum concentrations of CML among women with reduced GFR in this study were similar to CML concentrations described adults with diabetic nephropathy but less than levels described in diabetics with retinopathy. In contrast with the present study, a previous study in adults with diabetic nephropathy did not find that serum CML concentrations were predictive of adverse renal outcomes (Busch et al., Am J Kidney Dis 2006; 48: 571-579). The differences between the two studies may be due to the selection criteria involved in the respective studies. AGEs have been implicated in the pathogenesis of diabetic nephropathy and complications of end-stage renal disease (Vlassara et al., Proc Natl Acad Sci USA 91:11704-11708, 1994). AGEs upregulate inflammation and the synthesis of fibronectin, laminin, and collagen IV in the kidney and promote glomerular sclerosis, fibrosis, and hypertrophy. The kidney is affected by AGEs, and declining renal function entails an increase in serum AGEs, thereby amplifying damage from AGEs. AGEs are not merely a marker of renal insufficiency, as treatment with AGE inhibitors improves renal function, suggesting a direct role of AGEs in the pathogenesis of reduced GFR (Bolton et al., Am J Nephrol 24:32-40, 2004; Williams et al., Am J Nephrol 27:605-614, 2007). This is in contrast to what has been shown with hyperhomocysteinemia in kidney disease, where levels rise with declining renal function, but treatment has not been shown to be substantially beneficial (Bostom A. J Am Soc Nephrol 11: 149-151, 2000; Jamison et al., JAMA 298: 1212-1214, 2007).

Dietary intake of AGEs was not assessed in the present study, however, dietary intake of AGEs has been shown to correlate well with serum CML concentrations. The present study may underestimate the proportion of women who developed reduced GFR, as a separate analysis has shown that women with the lowest CML concentrations were at a considerable higher risk of mortality (Semba, submitted for publication). In the present study, serum CML was measured in non-fasting blood samples, and the post-prandial state may affect the concentrations of AGEs. Angiotensin-converting enzyme-1 inhibitors are also another factor that may potentially modulate the AGE-RAGE pathway.

In conclusion, elevated CML, a dominant AGE, and elevated circulating RAGE are associated with reduced GFR and appear to be predictive of the development of reduced GFR.

### Example 2

### Elevated Serum Advanced Glycation End Products and Poor Grip Strength in Older Community-Dwelling Women

Advanced glycation end products (AGEs) have been implicated in the pathogenesis of diabetes, heart disease, and kidney failure, and may potential affect skeletal muscle. Whether AGEs are associated with poor muscle strength is unknown.

About one-third of women and one-half of men ≧60 years in the United States are estimated to have sarcopenia, defined as the loss of skeletal muscle mass and strength with aging. With aging, there is a decrease in muscle cross-sectional area, loss of muscle fibers, and muscle fiber atrophy. Humans lose about 20% to 40% of both skeletal muscle mass and strength from 20 to 80 years of age. Low skeletal muscle mass is associated with low strength, decreased lower extremity performance, functional impairment, falls, and physical disability. Hand grip strength is strongly correlated with other measures of muscle strength and therefore is often considered representative of total body muscle strength. Hand grip strength is predictive of incident disability and long-term mortality.

The pathogenesis of sarcopenia has been attributed to undernutrition, oxidative stress, inflammation, endocrine changes, and inactivity. Low circulating levels of antioxidant nutrients such as carotenoids and selenium are associated with poor grip strength and impaired physical performance.

The relationship between serum AGEs and circulating RAGE and muscle strength in older adults has not been characterized. It was hypothesized in the present studies that elevated serum AGEs are associated with poor muscle strength. In order to address this hypothesis, serum AGE and circulating RAGE were measured in older women living in the community.

Study Participants

A cross-sectional study was conducted among 559 women, aged 65 and older, from the Women's Health and Aging Studies (WHAS) I, representative of the one-third most disabled women residing in the community in Baltimore, Md. Recruitment and exclusion criteria are discussed in Example 1. Further details on the methods and sampling design of the WHAS studies are published elsewhere (Guralnik et al., The Women's Health and Aging Study: Health and Social Characteristics of Older Women with Disability. Bethesda, Md., National Institute on Aging. NIH Publication No. 95-4009, 1995).

Data Collection

Data collected on the subjects is described above. Standardized questionnaires were administered in the participant's home by trained interviewers including questions regarding race, current smoking status, and education. A physical examination was performed and upon authorization blood was drawn. The definitions for the chronic diseases reported in this study were adjudicated by WHAS co-investigators based on standardized algorithms that combined information from the questionnaire, physical examination, and physician contact (Guralnik et al., 1995). The Mini-Mental Status Examination (MMSE) was administered at enrollment (Folstein et al., J Psychiatr Res 12:189-198, 1975). Women were seen every 6 months for a follow-up visit for 36 months, and phlebotomy was repeated at the 12 and 24 month follow-up visits. Further details on the methods and sampling design of the WHAS studies are published elsewhere (Guralnik et al., 1995).

Laboratory Studies

There were 1002 women enrolled in the Women's Health and Aging Study I. Eight hundred seventy-nine women returned for the 12-month follow-up visit, of whom 580 participated in the blood drawing. The 559 women involved in the present study were significantly younger, and a higher proportion had MMSE score <24, level of education <12 years, and stroke compared with the 320 women who are not included in the present analysis. Analyses of serum AGEs, sRAGE, and esRAGE were done at the 12-month follow-up visit rather than at enrollment because of a greater availability of serum from this visit. Non-fasting blood samples were obtained by venipuncture between 9 AM and 2 PM. Processing, aliquoting, and freezing were carried out at the Core Genetics Laboratory of The Johns Hopkins University School of Medicine following a standardized protocol. Blood samples were delivered to Quest Diagnostics Laboratories (Teterboro, N.J.) and in part stored continuously at −70° C. until the time of analyses for serum AGEs and circulating RAGE.

The measure of serum AGEs in this studywas serum carboxymethyl-lysine (CML) as discussed in Example 1. CML was measured using a competitive ELISA (AGE-CML ELISA, Microcoat, Penzberg, Germany) as described above. Total sRAGE was measured using a sandwich ELISA (Quantikine Human RAGE Immunoassay, R & D Systems, Minneapolis, Minn.) as described above. This assay measures C-truncated RAGE that has been enzymatically cleaved from the cell surface as well as esRAGE. Serum esRAGE was measured using ELISA (B-Bridge International, Mountain View, Calif.). Measurements were all performed in duplicate according the protocol of the manufacturers, and the results were averaged. The inter-assay coefficients of variation (CVs) for serum CML, sRAGE, and esRAGE were 4%, 7%, and 8%, respectively.

Serum carotenoids and serum selenium were included in these analyses because low levels of these nutrients have been previously associated with poor grip strength in this cohort. Serum carotenoids were measured by high performance liquid chromatography (Semba et al., Aging Clin Exp Res 2003; 15: 482-487, incorporated herein by reference). Total carotenoids were calculated as the sum of α-carotene, β-carotene, β-cryptoxanthin, lutein, zeaxanthin, and lycopene in μmol/L. The inter-assay CVs for α-carotene, β-carotene, β-cryptoxanthin, lutein, zeaxanthin, and lycopene were 12%, 8%, 4%, 12%, 12%, and 7%, respectively. Plasma selenium was measured by graphite furnace atomic absorption spectrometry using a Perkin Elmer AAnalyst 600 with Zeeman background correction. Samples were diluted 1:4 with a triton-X (Sigma Chemical, St. Louis, Mo.) and nitric acid solution (Fisher Scientific, Pittsburgh, Pa.), and the matrix modifier was a palladium and magnesium nitrate solution (both Perkin Elmer, Norwalk, Conn.). The instrument was calibrated daily using known plasma selenium standards (UTAK Laboratories, Inc., Valencia, Calif.). The inter-assay CV for selenium was 2%.

Statistical Analysis

Categorical variables were compared using chi-square tests. Body mass index (BMI) and MMSE score values were defined as above. Linear regression analysis was used to examine the relationship between serum CML, sRAGE, and esRAGE and other factors with grip strength as a continuous outcome variable. Variables that were at a level of significance of P<0.10 in univariate analyses were included in the multivariate models, except for hemoglobin Alc due to 144 missing values of this laboratory measure. Spearman correlations were used to examine correlations between serum CML, sRAGE, and esRAGE. The statistical program used was SAS (SAS Institute, Cary, N.C.).

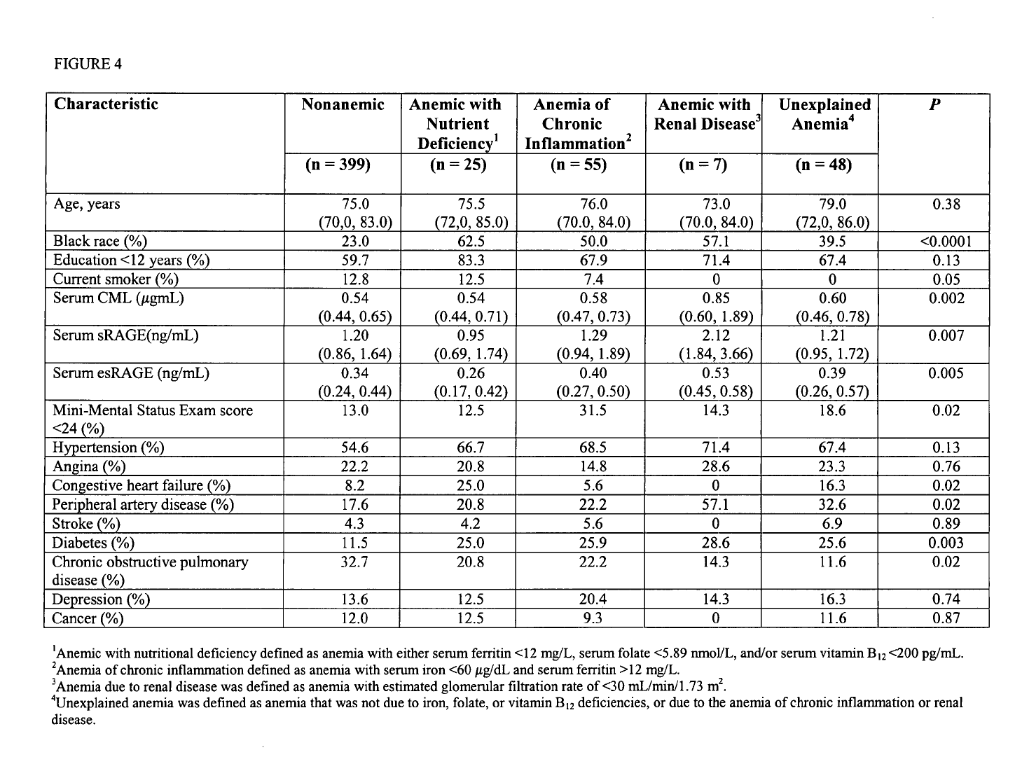

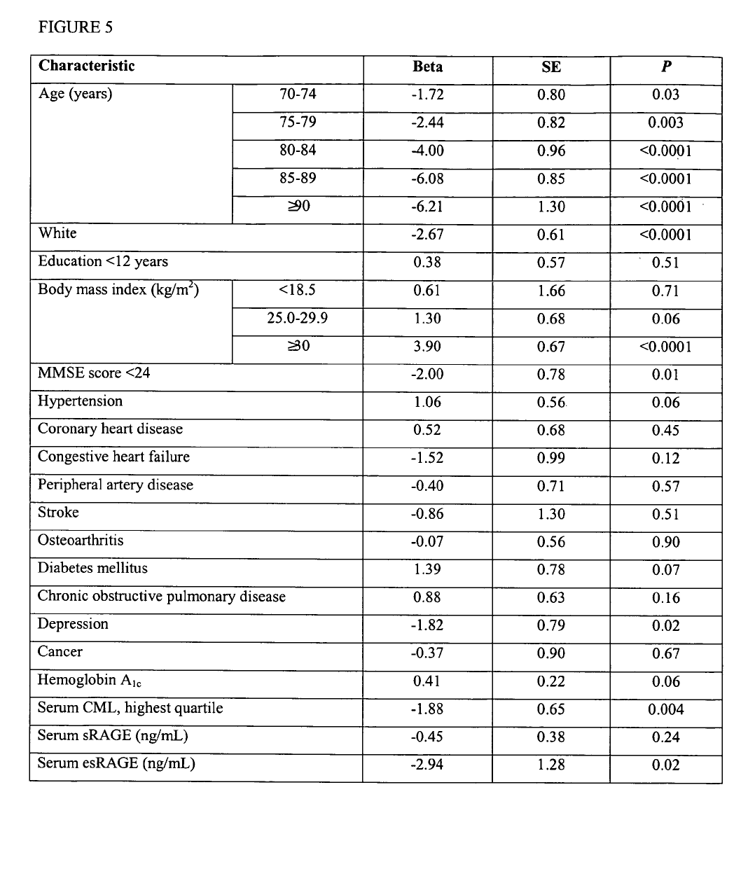

Demographic and disease characteristics of the 559 study participants from WHAS I are shown in the Table in FIG. 4. Overall, mean (SD) grip strength was 19.7 (6.3) kg. In univariate analyses, grip strength was significantly associated with age, race, BMI, MMSE<24, depression, serum CML, and esRAGE. Grip strength was not significantly associated with current smoking, education, serum sRAGE, hypertension, coronary heart disease, congestive heart failure, peripheral artery disease, stroke, osteoarthritis, chronic obstructive pulmonary disease, or cancer. Spearman correlations between serum CML and sRAGE and esRAGE, respectively, were r=0.18 and r=0.18 (both P<0.0001) and between sRAGE and esRAGE was r=0.89 (P<0.0001).

The Table shown in FIG. 5 shows univariate linear regression models of serum carboxymethyl-lysine and other factors with grip strength.

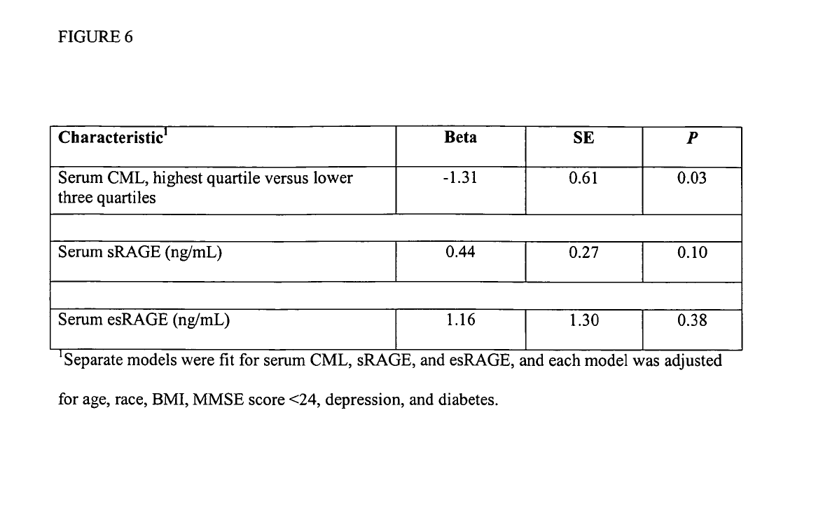

Exploratory analyses of different percentiles identified a deflection of the regression line between serum CML and grip strength, and this point coincided with the upper quartile of serum CML. The quartile cut-offs for serum CML were 0.45, 0.55, and 0.68 pg/mL. Quadratic terms were examined and were not significant. Mean (SD) grip strength among women in the highest quartile of serum CML compared with the lower three quartiles was 18.2 (6.4) and 20.1 (6.2) kg, respectively (P=0.004). Women in the top quartile of serum CML had a significantly higher risk of poor grip strength compared to women in the lower three quartiles in a multivariate linear regression analysis adjusting for age, race, body mass index, MMSE<24, depression, and diabetes (see Table in FIG. 6). Mean grip strength in women in the highest quartile of serum CML versus women in the lower three quartiles, was 18.6 and 20.0 kg, respectively (P=0.002), after adjusting for the same covariates in the Table shown in FIG. 6.

Exploratory analyses did not show a threshold between serum sRAGE and grip strength, and serum esRAGE and grip strength. Both serum sRAGE and serum esRAGE, respectively, were not significantly associated with grip strength in multivariate linear regression analyses adjusting for age, race, body mass index, MMSE<24, and depression (see Table shown in FIG. 6). There were no significant interactions between serum CML, sRAGE, or esRAGE, respectively, with race.

In order to determine whether total carotenoids and selenium were independently associated with grip strength, we entered both total carotenoids and selenium into the same multivariate model. Total carotenoids (mon) (beta=0.59, SE=0.21, P=0.005) and highest quartile of AGEs (pg/mL) (beta=−1.62, SE=0.62, P=0.009) were associated with grip strength in a multivariate analysis adjusting for age, race, body mass index, MMSE<24, and depression. Serum selenium (μg/dL) was not associated with grip strength in the same model (beta=0.015, SE=0.011, P=0.18).

The results presented here show that moderately to severely disabled older women living in the community with elevated serum AGEs have poor grip strength. To our knowledge, this is the first study to show an association between elevated serum AGEs and poor skeletal muscle strength in humans. This observation is consistent with the hypothesis that AGEs play a role in sarcopenia. Increased AGEs may contribute to increased stiffness in muscle tissue and reduced viscoelastic properties of muscle and thus impair muscle function. In rats, AGEs accumulate in skeletal muscle with aging. AGEs are known to increase blood vessel stiffness and bone rigidity through cross-linking of collagen. AGEs also accumulate in endothelial cells, where they contribute to endothelial dysfunction and upregulate inflammation through RAGE. Thus, AGE-related inflammation could contribute to loss of myocytes, and through this pathway, to loss of muscle mass and strength.

Both elevated serum AGEs and low serum carotenoids were independently associated with poor grip strength. Serum carotenoids are considered the strongest indicator of fruit and vegetable intake. The findings from this study suggest that two potentially modifiable dietary risk factors are associated with skeletal muscle strength. A limitation of this study is that causality cannot be strongly inferred in a cross-sectional study. It is possible that older women with poor grip strength were physically less able to have access to a more healthy diet, i.e., greater intake of fruits and vegetables and lower intake of foods processed at very high temperatures. The relationship between serum AGEs and skeletal muscle strength and physical performance needs to be examined in prospective studies to determine whether elevated serum AGEs predict a decline in skeletal muscle strength.

Circulating RAGE was not associated with grip strength. It is possible that circulating RAGE may be more strongly related to other systemic processes than those that affect skeletal muscle. The associations between sRAGE, esRAGE, and grip strength were in the same direction as serum AGEs, and it is also possible that larger sample size and power are needed to examine the association between circulating RAGE and skeletal muscle strength.

The present study was conducted among older, moderate to severely disabled women living in the community. The association between serum AGEs and grip strength was observed in a population of disabled women with mean grip strength of 19.7 kg, which is relatively low when compared with mean grip strength of 26.4 kg observed in a population-based sample of men and women.

In summary, serum AGEs were independently associated with grip strength, an observation which is consistent with the general concepts that AGEs may alter the structural property of tissues, including skeletal muscle, and contribute to muscle damage through the RAGE pathway and increased inflammation.

### Example 3

### Advanced Glycation End Products and their Circulating Receptors Predict Cardiovascular Disease Mortality in Older Community-Dwelling Women

The AGE-RAGE pathway has been the focus of growing interest because of substantial improvement in measurement technology and because experiments conducted in animal models have shown that blockage of AGE-RAGE binding reduces complications of atherosclerosis and diabetes.18 In humans, treatment with AGE-breakers and dietary restriction of AGE-containing foods improved cardiovascular function. A hypothesis in this study was that elevated levels of serum AGE, sRAGE, and esRAGE were predictive of mortality, especially cardiovascular disease mortality, in older persons. To address this hypothesis, AGE, sRAGE, and esRAGE were examined in a prospective study of older women living in the community.

Participants

A cross-sectional study was conducted among 559 women, aged 65 and older, from the Women's Health and Aging Studies (WHAS) I, representative of the one-third most disabled women residing in the community in Baltimore, Md. Recruitment and exclusion criteria are discussed in Example 1. Further details on the methods and sampling design of the WHAS studies are published elsewhere (Guralnik et al., The Women's Health and Aging Study: Health and Social Characteristics of Older Women with Disability. Bethesda, Md., National Institute on Aging. NIH Publication No. 95-4009, 1995).

Data Collection

Data collected on the subjects is described above. Standardized questionnaires were administered in the participant's home by trained interviewers including questions regarding race, current smoking status, and education. A physical examination was performed and upon authorization blood was drawn. The definitions for the chronic diseases reported in this study were adjudicated by WHAS co-investigators based on standardized algorithms that combined information from the questionnaire, physical examination, and physician contact (Guralnik et al., 1995). The Mini-Mental Status Examination (MMSE) was administered at enrollment (Folstein et al., J Psychiatr Res 12:189-198, 1975). Women were seen every 6 months for a follow-up visit for 36 months, and phlebotomy was repeated at the 12 and 24 month follow-up visits. Further details on the methods and sampling design of the WHAS studies are published elsewhere (Guralnik et al., 1995).

Vital status was determined through matching with the National Death Index from the 12 month follow-up visit, 1993-1996 through the end of 2000. Causes of death as coded by the International Classification of Diseases-9 were recorded (International Classification of Diseases. Ninth revision, clinical modification. Washington, D.C., U. S. Health and Human Services, Centers for Disease Control and Prevention, Centers for Medicare and Medicaid Services, 2006). The Johns Hopkins University Institutional Review Board approved the study protocol, and written informed consent was obtained from all participants.

Laboratory Studies

There were 1002 women enrolled in the Women's Health and Aging Study I, of whom 746 women participated in the baseline blood drawing. Eight hundred seventy-nine women participated in the 12-month follow-up visit, of whom 580 received a blood draw. Analyses of serum AGEs, sRAGE, and esRAGE were done at the 12-month follow-up visit rather than at enrollment because of a greater availability of serum aliquots in the sample repository from this visit. The 559 women involved in the present study were significantly younger, and a higher proportion had MMSE score <24, level of education <12 years, stroke, and stroke compared with the 320 women who are not included in the present analysis. Non-fasting blood samples were obtained by venipuncture between 9 AM and 2 PM. Processing, aliquoting, and freezing were carried out at the Core Genetics Laboratory of The Johns Hopkins University School of Medicine following a standardized protocol. Blood samples were stored continuously at −70° C. until the time of analyses of serum AGEs, sRAGE, and esRAGE.

The measure of serum AGEs in this study was serum carboxymethyl-lysine (CML). CML is a dominant circulating AGE, the best characterized of all the AGEs, and a dominant AGE in tissue proteins. Measurements of AGE, sRAGE, and esRAGE were performed as described above: CML was measured using a competitive ELISA (AGE-CML ELISA, Microcoat, Penzberg, Germany); total sRAGE was measured using a sandwich ELISA (Quantikine Human RAGE Immunoassay, R & D Systems, Minneapolis, Minn.); and C-truncated RAGE that has been enzymatically cleaved from the cell surface as well as esRAGE. Serum esRAGE was measured using ELISA (B-Bridge International, Mountain View, Calif.). Measurements were all performed in duplicate according the protocol of the manufacturers, and the results were averaged. The within assay and between assay coefficients of variation (CVs) for serum AGE, sRAGE, and esRAGE were 3% and 4%, 3% and 7%, and 6% and 8%, respectively.

Data Analysis

Continuous variables were compared using Wilcoxon rank-sum test. Body mass index (BMI) and MMSE score values were defined as above. Renal insufficiency was defined as estimated glomerular filtration rate of <60 mL/min/1.73 m² using the Modification of Diet in Renal Disease equation of Levey and colleagues (35). Cardiovascular disease mortality was defined by the death codes 390-459 from the 9th version of the International Classification of Diseases (ICD) (30). Cox proportional hazards models were used to examine the relationship between serum CML, sRAGE, and esRAGE, and 4.5 year all-cause and cardiovascular disease mortality. Variables that were significant in the univariate” analyses were entered into the multivariate Cox proportional hazards models, except in the situation where the variables were known to be in the causal pathway, i.e., congestive heart failure and peripheral artery disease. Survival curves were compared using log-rank test. The statistical program used was SAS (SAS Institute, Cary, N.C.), with data analysis conducted by Kai Sun. The level of significance used in this study was P<0.05.

During 4.5 years of follow-up, 123 of 559, or 22%, of women died. The main causes of death were cardiovascular disease (43.9%), cancer (17.9%), chronic obstructive pulmonary disease (5.7%), pneumonia (4.9%), urinary tract infection (3.3%), diabetes mellitus (1.6%), renal disease (1.6%), sepsis (1.6%), and other (20.3%).

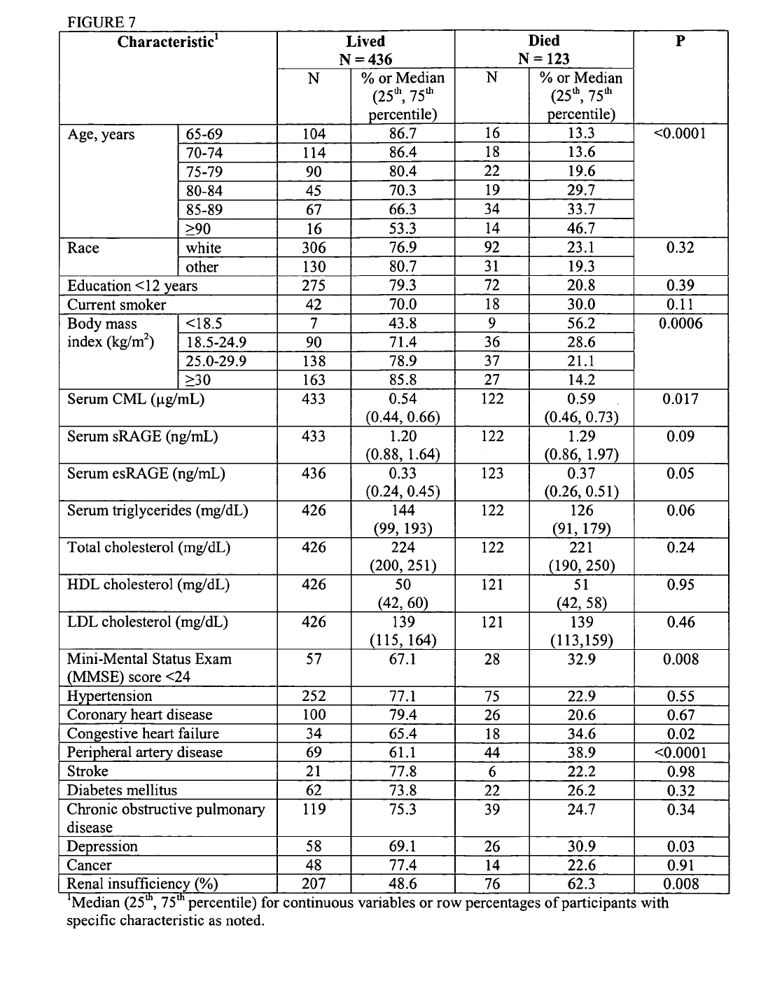

Demographic and other characteristics of women who died from all causes or survived are shown in the Table in FIG. 7. Median serum CML and serum esRAGE concentrations were significantly higher in women who died from all causes compared to women who survived. Serum sRAGE concentrations were higher in women who died from all causes compared to women who survived (P=0.09). Women who died from all causes were older, had lower body mass index, and were more likely to have cognitive impairment, congestive heart failure, peripheral artery disease, depression, and renal insufficiency. There were no significant differences between women who survived or died from all causes by race, education <12 years, current smoking, triglycerides, total cholesterol, .HDL cholesterol, LDL cholesterol, and prevalence of hypertension, coronary heart disease, stroke, diabetes, chronic obstructive pulmonary disease, or cancer.

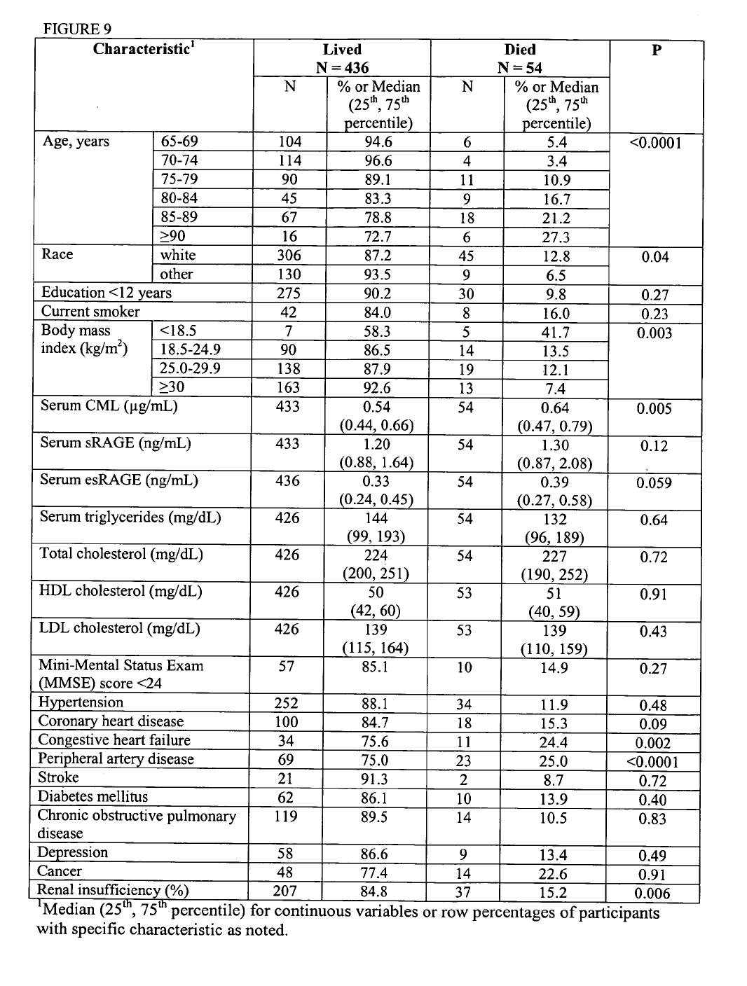

The demographic and other characteristics of women who died from cardiovascular diseases or survived are shown in the Table in FIG. 9. Median serum CML concentrations were significantly higher in women who died from cardiovascular disease compared to women who survived. Serum sRAGE and esRAGE concentrations were higher in women who died from cardiovascular disease compared to women who survived, a finding which did not reach statistical significance (P=0.12, P=0.059, respectively). Women who died from cardiovascular disease were older, less likely to be overweight and obese, and were more likely to be white and to have congestive heart failure, peripheral artery disease, and renal insufficiency. There were no significant differences between women who survived or died from all causes by education <12 years, current smoking, triglycerides, total cholesterol, HDL cholesterol, LDL cholesterol, MMSE<24, and prevalence of hypertension, coronary heart disease, stroke, diabetes, chronic obstructive pulmonary disease, depression, or cancer.

Serum CML and Mortality

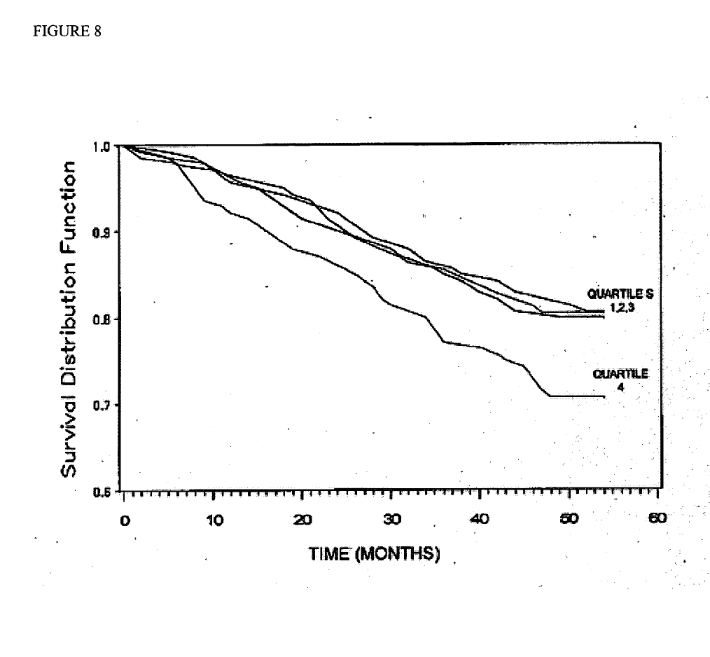

The relationship between serum CML and all-cause and cardiovascular disease mortality was examined using CML as quartiles since there was a threshold at the highest quartile. The survival curves for women in each quartile of serum CML and all-cause mortality are shown in FIG. 8. Quartile cut-offs for serum CML were 0.45, 0.55, and 0.68 pg/mL. The proportion of women who died from all causes in each quartile, from lowest to highest, were 19.3%, 19.3%, 20.0%, and 29.3%, respectively. Women in the highest quartile of serum CML had lower survival than women in the lower three tertiles (P=0.013, log-rank test). Women in the highest quartile of serum CML had an increased risk of dying from all causes compared to women in the lower three quartiles (H.R. 1.47, 95% C.I. 0.97-2.22, P=0.066) in a multivariate Cox proportional hazards model, adjusting for age, BMI, MMSE<24, depression, and renal insufficiency (see Table shown in FIG. 9).

The survival curves for cardiovascular disease mortality are shown for women in each quartile of serum CML in FIG. 11. The proportion of women who died from cardiovascular disease in each quartile, from lowest to highest, were 8.9%, 8.2%, 8.2%, and 18.9%, respectively. Women in the highest quartile of serum CML had lower survival than women in the lower three tertiles (P=0.0009, log-rank test). Women in the highest quartile of serum CML had an increased risk of dying from cardiovascular disease compared to women in the lower three quartiles (Hazards Ratio [H.R.] 1.94, 95% Confidence Interval [C.I.] 1.08-3.48, P=0.026) in a multivariate Cox proportional hazards model, adjusting for age, BMI, and renal insufficiency (Table 4). There were no significant interactions between serum CML and diabetes in relation to either all-cause or cardiovascular disease mortality.

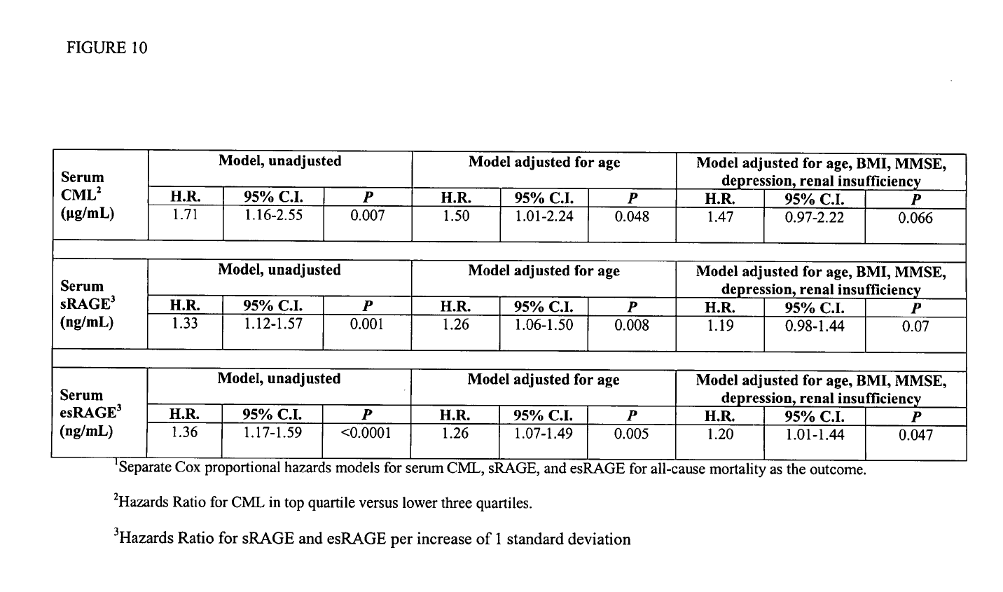

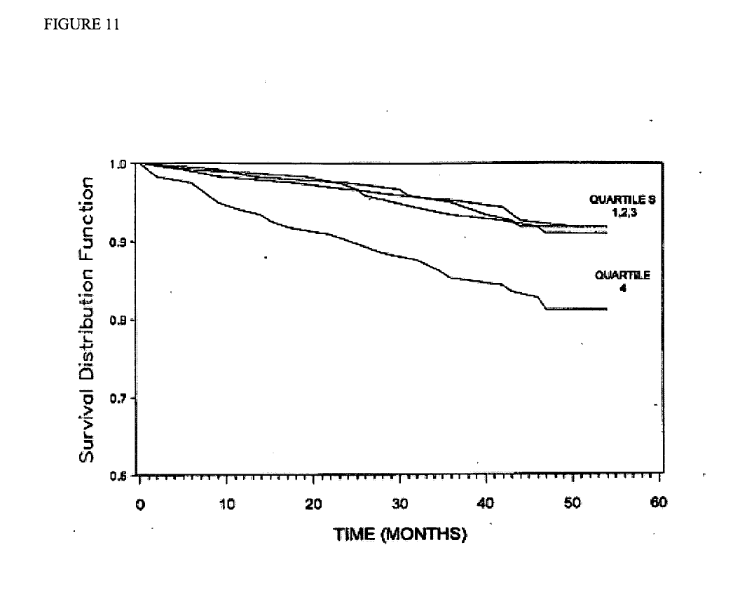

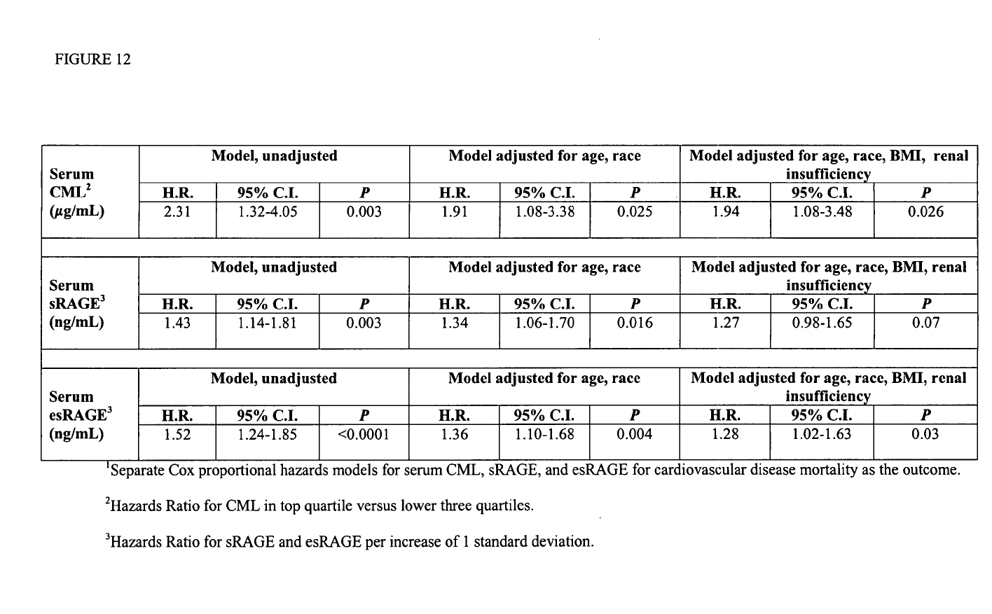

The relationship between circulating RAGE (esRAGE, sRAGE) was examined as a continuous variable only, as exploratory analyses of quartiles did not show that the highest quartile or quartiles had strong relationships with mortality as found with the analysis of serum CML. Serum sRAGE (ng/mL) was predictive of all-cause mortality (H.R. per 1 SD increase, 1.19, 95% C.I. 0.98-1.44, P=0.07) in a multivariate Cox proportion hazards model after adjusting for age, body mass index, MMSE<24, depression, and renal insufficiency (FIG. 10). Total sRAGE (ng/mL) predicted cardiovascular disease mortality (H.R. per 1 S.D. increase, 1.27, 95% C.I. 0.98-1.65, P=0.07), adjusting for age, BMI, and renal insufficiency (Table in FIG. 12). There were no significant interactions between serum sRAGE and diabetes in relation to either all-cause or cardiovascular disease mortality.

Serum esRAGE (ng/mL) was predictive of all-cause mortality (H.R. per 1 S.D. increase, 1.20, 95% C.I. 1.01-1.44, P=0.047), adjusting for age, body mass index, MMSE<24, depression, and renal insufficiency (Table in FIG. 10). Serum esRAGE (ng/mL) was predictive of cardiovascular disease mortality (H.R. per 1 S.D. increase, 1.28, 95% C.I. 1.02-1.63, P=0.03), after adjusting for age, race, BMI, and renal insufficiency. There were no significant interactions between serum esRAGE and diabetes in relation to either all-cause or cardiovascular disease mortality.

There were 84 women with diabetes and 475 women without diabetes. Median (25th, 75th percentile) serum CML among women with and women without diabetes was 0.52 (0.42, 0.67) and 0.55 (0.45, 0.68) pg/mL, respectively (P=0.06). Median (25th, 75th percentile) serum sRAGE among women with and women without diabetes was 1.20 (0.80, 1.67) and 1.20 (0.80, 1.67) ng/mL, respectively (P=0.58). Median (25th, 75th percentile) serum esRAGE among women with and without diabetes was 0.32 (0.23, 0.46) and 0.35 (0.25, 0.46) ng/mL, respectively (P=0.32).

In order to examine the relationship between serum CML and RAGE with mortality in non-diabetic women, analyses were conducted in which the 84 women with diabetes were excluded. Among non-diabetic women, serum CML, sRAGE, and esRAGE, respectively, predicted all-cause mortality (H.R. for highest versus lower three quartiles, 1.81, 95% C.I. 1.17-2.82, P=0.008; H.R. per 1 S.D., 1.14, 95% C.I. 0.91-1.42, P=0.26; H.R. per 1 S.D. 1.22, 95% C.I. 0.097-1.53, P=0.09, after adjusting for age, body mass index, MMSE<24, depression, and renal insufficiency. Among non-diabetic women, serum CML, sRAGE, and esRAGE, respectively, predicted cardiovascular disease mortality (H.R. for highest versus lower three quartiles, 2.29, 95% C.I. 1.21-4.34, P=0.01; H.R. per 1 S.D., 1.24, 95% C.I. 0.92-1.65, P=0.16; H.R. per 1 S.D. 1.45, 95% C.I. 1.08-1.93, P=0.01, after adjusting for age, race, body mass index, and renal insufficiency.

These results suggest that moderately to severely disabled older, community-dwelling women with elevated serum AGEs are at a greater risk of dying, especially from cardiovascular diseases. In addition, women with elevated circulating RAGE were at an increased risk of cardiovascular disease mortality. The two major sources of systemic AGEs are thought to be endogenous AGEs, generated by abnormal glucose metabolism, and exogenous AGEs found in foods.

Most studies of AGEs and their circulating receptors have been limited to patients with specific diseases, mainly diabetes, atherosclerosis, and end-stage renal disease. The strengths of this study were a relatively large population-based sample of community-dwelling older women and measurements of both serum AGE and circulating RAGE. These results are consistent with previous studies showing that elevated serum AGEs predicted mortality in hemodialysis patients (41) and cardiovascular mortality in women with type 2 diabetes (3). In the present study, elevated serum AGEs predicted both all-cause and cardiovascular disease mortality in women without diabetes. The biological mechanisms by which elevated AGEs could increase the risk of dying cannot be specifically determined from this epidemiological study. However, there is potential for elevated AGEs to cause widespread damage to multiple systems, as AGEs are known to alter the structural quality of blood vessels, bone, skeletal muscle, and other tissues through cross-linking with collagen and to accelerate inflammation, atherosclerosis, and renal damage through the AGE-RAGE pathway.

In the present study, women with elevated circulatory RAGE, both total sRAGE and esRAGE, were at an increased risk of cardiovascular death. In contrast, previous studies showed that low plasma esRAGE was a predictor of cardiovascular mortality in patients with end-stage renal disease (Wagner et al., Am J Kidney Dis 2006; 47: 294-300). The differences in these findings may be due to the contrasting clinical characteristics of the two study populations. Whether elevated circulating RAGE concentrations are a biological response that allows circulating RAGE to bind circulating AGE and thus prevent AGE from binding with membrane-bound RAGE is not known. Circulating RAGE may be insufficient to antagonize AGE-RAGE interactions because RAGE concentrations in plasma are 1000 times lower than circulating AGEs (Vlassara and Palace, J Intern Med 2002; 251: 87-101). However, the ratio of circulating RAGE to AGE may be much different at the localized sites where RAGE is upregulated than in the general circulation. Although sRAGE was a significant predictor of mortality, sRAGE consists of both esRAGE and cleaved isoforms of RAGE. The cleaved isoforms of RAGE alone were not significantly predictive of mortality, suggesting that circulating esRAGE may be a more important biological marker for mortality than cleaved isoforms of RAGE.

In this study, elevated serum AGEs, sRAGE, and esRAGE were predictive of cardiovascular disease mortality, and appeared to be predictive of all-cause mortality at a level of marginal significance. The magnitude of the hazards ratios for mortality was greater for cardiovascular disease mortality than all-cause mortality for serum AGE, sRAGE, and esRAGE. These findings suggest that elevated AGE and its receptors may be more specifically involved in cardiovascular disease mortality. The association between serum CML and both all-cause and cardiovascular disease mortality appeared to be non-linear, with a threshold for the highest quartile. These findings suggest that there may be a critical threshold for AGEs, above which the risk of mortality increases greatly.

### Example 4

### Association of Serum Carboxymethyl-Lysine, a Dominant Advanced Glycation End Product, with Anemia in Adults

Anemia is common in older adults, and the prevalence of anemia increases with advancing age. Anemia has been associated with a wide spectrum of adverse outcomes, including reduced quality of life, decreased muscle strength, increased disability, cognitive impairment, higher risk of Alzheimer disease, and increased mortality. The reduction of oxygen-carrying capacity of the blood that occurs with anemia may account for fatigue, cardiovascular complications, and impaired physical performance.

The pathophysiology of anemia in older adults is incompletely understood, and a substantial proportion of anemia in this population remains unexplained. The factors that might play a role in anemia in adults have not been completely identified.

Carboxymethyl-lysine (CML) is a dominant AGE that accumulates in large arteries, kidney, muscle, bone, and erythrocytes, and CML can lead to the formation of highly reactive dicarbonyl compounds that react with proteins and propagate intramolecular or intermolecular cross-link formation. CML progressively accumulates within erythrocytes during their life span in the circulation. AGEs reduce the deformability of erythrocytes, an effect that can be reversed by AGE inhibitors. AGEs on the surface of erythrocytes increase the binding of erythrocytes to blood vessel walls through interactions with the receptor for AGEs (RAGE) on the endothelial surface. Altered deformability of erythrocytes induced by AGEs, and erythrocyte AGE-RAGE interactions could potentially shorten the life-span of erythrocytes and contribute to anemia. It has been demonstrated that erythropoietin levels increase with age in individuals who maintain a normal hemoglobin level, a finding that could reflect compensation for increased erythrocyte turnover. Unfortunately, data on erythrocyte life span in aging are not yet available.

A previous study described elevated serum AGEs in anemic patients with type 2 diabetes, but it is not clear whether elevated AGEs are associated with anemia in the general population. Here it is hypothesized that elevated serum AGEs were associated with anemia in adults. To examine this hypothesis, serum CML and anemia were characterized in a cohort of community-dwelling adults.

Study Population

The study subjects consisted of participants in the Baltimore Longitudinal Study of Aging (BLSA) who were seen between April 2002 and August. 2007. The BLSA is a prospective open cohort study of community-dwelling volunteers, largely from the Baltimore/Washington area. The study was established in 1958 and is described in detail elsewhere. 27 BLSA participants return periodically to the National Institute on Aging Clinical Research Center in Baltimore, Md., for 2.5 days of medical, physiological, and psychological examinations. Height, weight, and waist circumference were determined for all participants. Body mass index was determined as kg/m². Smoking status was ascertained by a questionnaire that classified each subject as a non-smoker, former smoker, or current smoker. Use of medications was determined at each study visit. The BLSA has continuing approval from the Institutional Review Board (IRB) of the MedStar Research Institute, and the protocol for the present study was also approved by the IRB of the Johns Hopkins School of Medicine.

Laboratory Studies

Complete blood count was conducted using a hematology analyzer (Coulter). Serum creatinine was measured using the Jaffe method. Blood samples were stored continuously at −70° C. until the time of analyses of serum AGEs. Serum carboxymethyl-lysine (CML) levels were used as the index measure of serum AGEs in this study. CML is a dominant circulating AGE, the best characterized of all the AGEs, and a dominant AGE in tissue proteins. CML was measured using a competitive ELISA (AGE-CML ELISA, Microcoat, Penzberg, Germany) (Boehm et al., Diabetologia 2004; 47:1376-1379). This assay has been validated (Zhang et al., Clin Chem Lab Med 2005; 43:503-511), is specific, and shows no cross-reactivity with other compounds. Measurements were all performed in duplicate according to the protocol of the manufacturers, and the results were averaged. The within assay and between assay coefficients of variation (CVs) for serum CML were both <5%.

Statistical Analysis

Continuous variables were compared using Wilcoxon rank-sum test. Categorical variables were compared using chi-square tests. Anemia was defined as per the World Health Organization definition of <12 g/dL in women and <13 g/dL in men. Renal insufficiency was defined as estimated glomerular filtration rate of <60 mL/min/1.73 m² using the Modification of Diet in Renal Disease equation of Levey and colleagues. Logistic regression models were used to examine the relationship between serum CML and other factors with anemia. Linear regression models were used to examine the same cross-sectional relationships where the dependent variable was hemoglobin. Variables that were significantly associated with anemia in univariate analyses were entered into multivariate logistic and linear regression models. In linear and logistic regression models, one standard deviation in concentration of serum CML was used as the unit of change. The statistical program used was SAS (SAS Institute, Cary, N.C.), with data analysis conducted by Kai Sun. The level of significance used in this study was P<0.05.

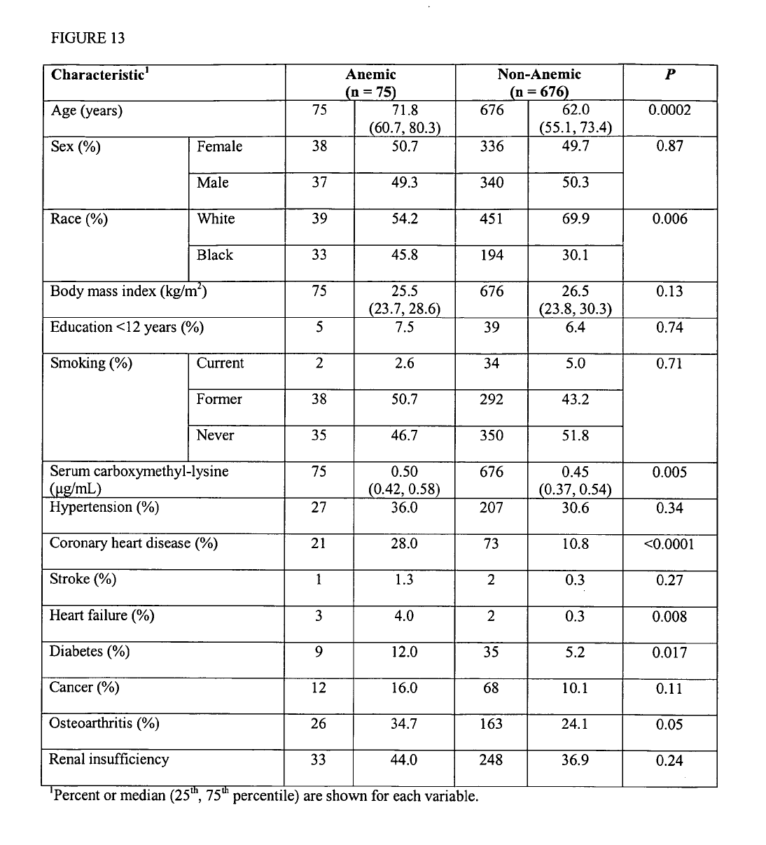

The demographic and health characteristics of 751 men and women with and without anemia are shown in the Table in FIG. 13. Overall, mean (SD) serum CML was 0.47 (0.13) μg/mL. Of 751 adults, 75 (10.0%) had anemia. Those with anemia were more likely to be older, black, and to have coronary heart disease, heart failure, and diabetes. There were no significant differences in sex, body mass index, education, or prevalence of hypertension, stroke, and cancer between those with and without anemia. The prevalence of heart failure and stroke were both less than 2% (Table in FIG. 13).

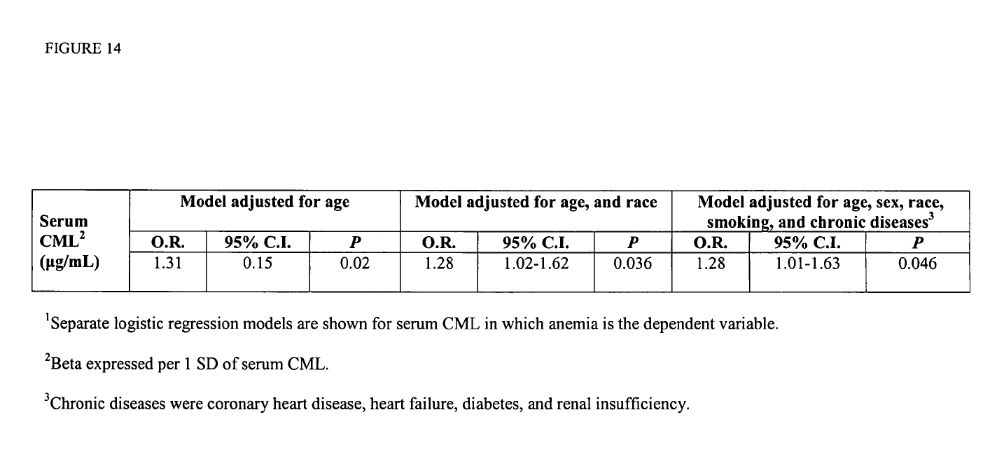

Separate multivariate logistic regression models were used to examine the cross-sectional relationship between serum CML and anemia (Table in FIG. 14). Serum CML was significantly associated with increased odds of anemia in models adjusted for age and sex, and additionally adjusted for race, coronary heart disease, heart failure, diabetes, and renal insufficiency (Table in FIG. 14).

In an alternative multivariate logistic regression model in which all subjects with diabetes were excluded, serum CML was associated with increased odds of anemia (0.R. per 1 S.D. 1.33, 95% C.I. 1.03-1.72, P=0.029) adjusting for age, sex, race, smoking, coronary heart disease, heart failure, and renal insufficiency.

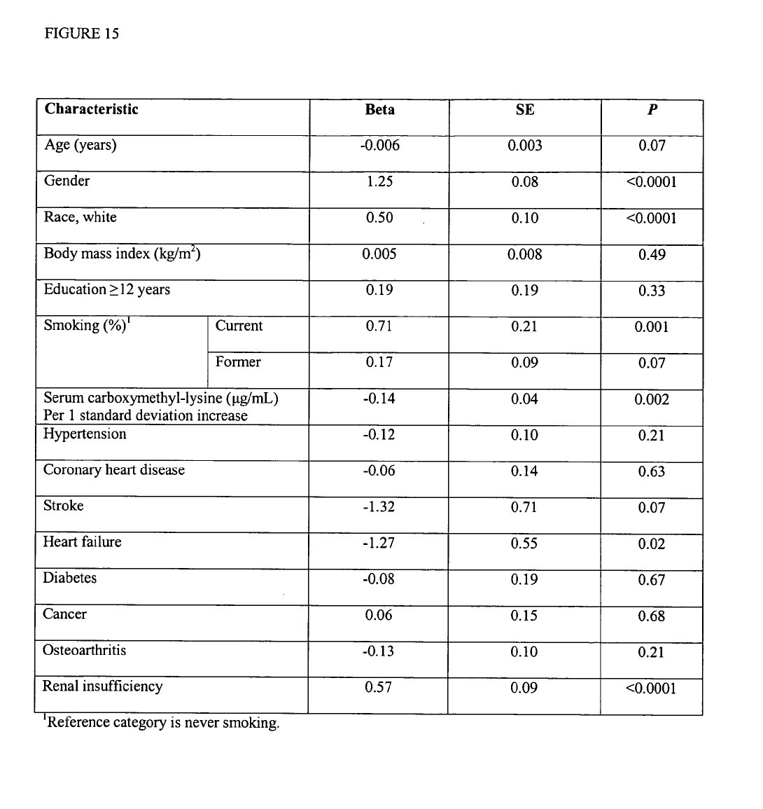

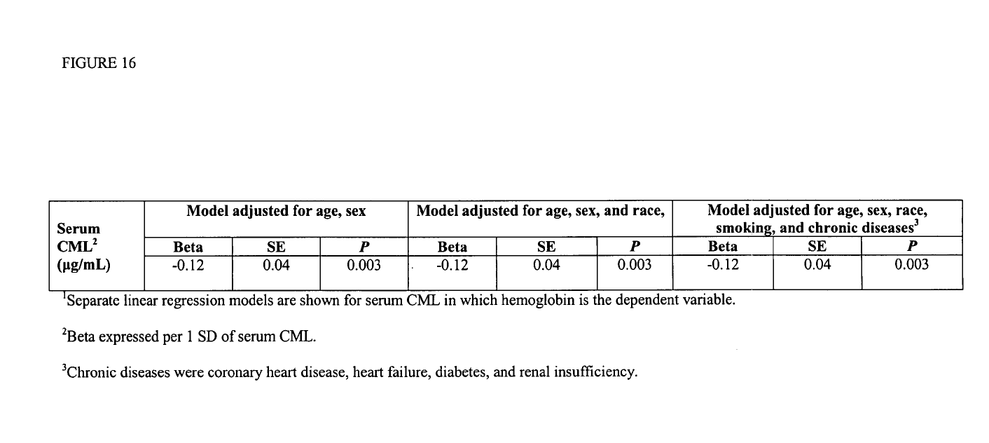

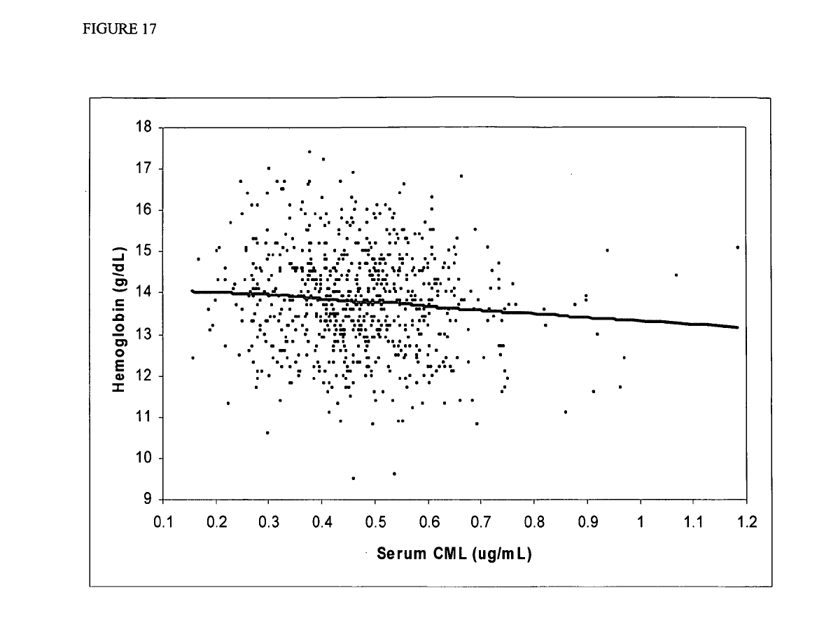

The relationship between serum CML and hemoglobin is shown in a scatterplot in FIG. 17. The relationship between serum CML and hemoglobin was examined in univariate linear regression analyses shown in the Table in FIG. 15. Age, race, current smoking, current smoking, serum CML, heart failure, and renal insufficiency were associated with hemoglobin. Body mass index, education, hypertension, coronary heart disease, stroke, diabetes, and cancer were not associated with hemoglobin. Serum CML was significantly and inversely associated with hemoglobin in separate multivariate linear regression models adjusting for age and sex, and additionally adjusted for race, and adjusting for age, sex, race, smoking, and chronic diseases (the Table in FIG. 16).

In an alternative multivariate linear regression model in which all subjects with diabetes were excluded, serum CML was associated with hemoglobin (beta per 1 S.D.−0.12, SE=0.04, P=0.002) adjusting for age, sex, race, smoking, coronary heart disease, heart failure, and renal insufficiency.

The present study suggests that elevated AGEs, as indicated by serum CML, are associated with anemia in community-dwelling adults. This is the first study to report an association between elevated AGEs and anemia in a population of community-dwelling adults. The present study is consistent with a previously reported association of elevated AGEs and anemia among patients with type 2 diabetes. Whether there is a causal relationship between elevated serum CML and anemia is not clear. As noted previously, CML alters the deformability of erythrocytes and increases interactions between erythrocytes and the endothelial surface via interactions of erythrocyte AGE with RAGE. In addition, CML forms adducts with hemoglobin, but whether the formation of hemoglobin-CML affects the lifespan of erythrocytes has not been determined.

The serum concentrations of CML among adults with anemia in this study were lower than CML concentrations described adults with diabetic nephropathy and diabetics with retinopathy, and this may be due to the lower prevalence of advanced diabetes and chronic diseases among participants in the BLSA compared with the two other study populations.

The design of the study was cross-sectional, and the direction of the association between AGEs and anemia cannot be determined. The study did not include the dietary assessment of AGEs, which must be conducted using a specialized questionnaire that addresses the method of food preparation. However, serum CML concentrations have been shown previously to correlate well with dietary intake of AGEs.

Serum AGEs are a potentially modifiable risk factor, as systemic levels of AGEs can be reduced substantially by decreasing dietary intake of AGEs by avoiding foods that are processed at high temperatures, i.e., deep fried, oven fried, grilled, and broiled. Administration of AGE-breakers or AGE inhibitors has been shown to reduce endothelial dysfunction and to improve cardiovascular and renal fimetion, but it is not known whether these pharmacological interventions will increase hemoglobin concentrations.

In conclusion, elevated CML, a dominant AGE, is independently associated with anemia in community-dwelling men and women.

### Example 5

### Elevated Serum Advanced Glycation End Products and their Circulating Receptors are Associated with Anemia in Older Community-Dwelling Women

Anemia is common in older adults, and the prevalence of anemia increases with advancing age. The factors that cause anemia in older persons have not been completely characterized. Advanced glycation end products (AGEs) are bioactive molecules implicated in the pathogenesis of renal insufficiency, diabetes, and atherosclerosis. AGEs are formed by the non-enzymatic glycation of proteins and other molecules. Recent studies suggest that AGEs accumulate in erythrocytes and alter their deformability. The decreased deformability induced by AGEs in erythrocytes is reversed by AGE inhibitors. The AGEs on the surface of erythrocytes can bind with receptor for AGEs (RAGE) on the vascular endothelium. A previous study described elevated serum AGEs in anemic patients with type 2 diabetes, but it is not clear whether elevated AGEs are associated with anemia in the general population.

The relationship between circulating forms of RAGE and anemia has not been characterized. There may be differences between concentrations of the two circulating receptors because esRAGE is expressed after transcriptional activation. Circulating RAGE can bind AGE and prevent AGE activation of cell membrane-bound RAGE. Circulating RAGE may serve as a decoy receptor to counteract the inflammatory processes triggered by RAGE ligands such as AGEs. Thus, in order to have greater insight into the role of AGEs and RAGE in relation to anemia, both AGEs and RAGE should be considered.

The AGE-RAGE pathway has been the focus of growing interest because of substantial improvement in measurement technology and because experiments conducted in animal models have shown that blockage of AGE-RAGE binding may reduce the deleterious effects of AGEs on disease. We postulated that elevated levels of serum AGE, sRAGE, and esRAGE were associated with anemia. To address this hypothesis, AGE, sRAGE, and esRAGE were characterized, and anemia in a cohort of older women living in the community.

Study Population

A cross-sectional study was conducted among 559 women, aged 65 and older, from the Women's Health and Aging Studies (WHAS) I, representative of the one-third most disabled women residing in the community in Baltimore, Md. Recruitment and exclusion criteria are discussed in Example 1. Further details on the methods and sampling design of the WHAS studies are published elsewhere (Guralnik et al., The Women's Health and Aging Study: Health and Social Characteristics of Older Women with Disability. Bethesda, Md., National Institute on Aging. NIH Publication No. 95-4009, 1995).

Data Collection

Data collected on the subjects is described above. Standardized questionnaires were administered in the participant's home by trained interviewers including questions regarding race, current smoking status, and education. A physical examination was performed and upon authorization blood was drawn. The definitions for the chronic diseases reported in this study were adjudicated by WHAS co-investigators based on standardized algorithms that combined information from the questionnaire, physical examination, and physician contact (Guralnik et al., 1995). The Mini-Mental Status Examination (MMSE) was administered at enrollment (Folstein et al., J Psychiatr Res 12:189-198, 1975). Women were seen every 6 months for a follow-up visit for 36 months, and phlebotomy was repeated at the 12 and 24 month follow-up visits. Further details on the methods and sampling design of the WHAS studies are published elsewhere (Guralnik et al., 1995).

At the 12-month follow-up visit, 879 women returned for follow-up, of which 580 received a blood draw. AGE and RAGE were measured in 519 women who had hemoglobin measurements available. The 519 women involved in the present study were significantly younger, and a higher proportion had MMSE score <24, level of education <12 years, and stroke compared with the 360 women who are not included in the present analysis. Laboratory measurements of serum AGEs, sRAGE, and esRAGE were done at the 12-month follow-up visit rather than at enrollment because of a greater availability of serum aliquots from this visit.

Laboratory Studies

Non-fasting blood samples were obtained by venipuncture between 9 AM and 2 PM. Blood samples were delivered to Quest Diagnostics Laboratories (formerly Ciba-Corning Laboratories, Baltimore, Md.) on the day of blood drawing for complete blood count, folate, vitamin B12, creatinine, and serum iron measurements. Serum creatinine was measured using the Jaffe method. Serum vitamin B12 and folate were measured by immunoassay (Stabler et al., Am J Clin Nutr 1999; 70:911-9). Processing, aliquoting, and freezing were carried out at the Core Genetics Laboratory of the Johns Hopkins University School of Medicine following a standardized protocol. Blood samples were stored continuously at −70° C. until the time of analyses of serum AGEs, sRAGE, and esRAGE.

The measure of serum AGEs in this study was serum carboxymethyl-lysine (CML). CML is a dominant circulating AGE, the best characterized of all the AGEs, and a dominant AGE in tissue proteins. Measurements of AGE, sRAGE, and esRAGE were performed as described above: CML was measured using a competitive ELISA (AGE-CML ELISA, Microcoat, Penzberg, Germany); total sRAGE was measured using a sandwich ELISA (Quantikine Human RAGE Immunoassay, R & D Systems, Minneapolis, Minn.); and C-truncated RAGE that has been enzymatically cleaved from the cell surface as well as esRAGE. Serum esRAGE was measured using ELISA (B-Bridge International, Mountain View, Calif.). Measurements were all performed in duplicate according to the protocol of the manufacturers, and the results were averaged. The within assay and between assay coefficients of variation (CVs) for serum CML, sRAGE, and esRAGE were 3% and 4%, 3% and 7%, and 6% and 8%, respectively.

Statistical Analysis

Continuous variables were compared using Wilcoxon rank-sum test. Categorical variables were compared using chi-square tests. Anemia was defined as hemoglobin <12 g/dL. Types of anemia were defined using a framework previously described (Semba et al., Aging Clin Exp Res 2007; 19:259-64, incorporated herein by reference). In brief, among women with hemoglobin <12 g/dL, iron deficiency anemia was defined as serum ferritin <12 mg/L, folate deficiency anemia was defined as serum folate <5.89 nmol/L, and anemia due to serum vitamin B12 deficiency was defined as serum vitamin B₁₂<200 pg/mL. Among anemic women, the anemia of chronic inflammation was defined as serum iron <60 μg/dL and serum ferritin >12 mg/L, and anemia due to renal disease was defined as anemia in the presence of an estimated glomerular filtration rate of <30 mL/min/1.73 m². Unexplained anemia was defined as anemia that was not due to iron, folate, or vitamin B₁₂ deficiencies, or due to the anemia of chronic inflammation or renal disease.

Body mass index (BMI) and MMSE score values were defined as above. Renal insufficiency was defined as estimated glomerular filtration rate of <60 mL/min/1.73 m² using the Modification of Diet in Renal Disease equation of Levey and colleagues. Logistic regression models were used to examine separately the relationships of serum CML, sRAGE, and esRAGE, with anemia. Linear regression models were used to examine the same cross-sectional relationships where the dependent variable was hemoglobin. Variables that were significant in the univariate analyses were entered into the multivariate logistic and linear regression models. In linear and logistic regression models, a one standard deviation (S.D.) in concentration of serum CML, sRAGE, and esRAGE, respectively, was used as the unit of change. The statistical program used was SAS (SAS Institute, Cary, N.C.), with data analysis conducted by Kai Sun. The level of significance used in this study was P<0.05.

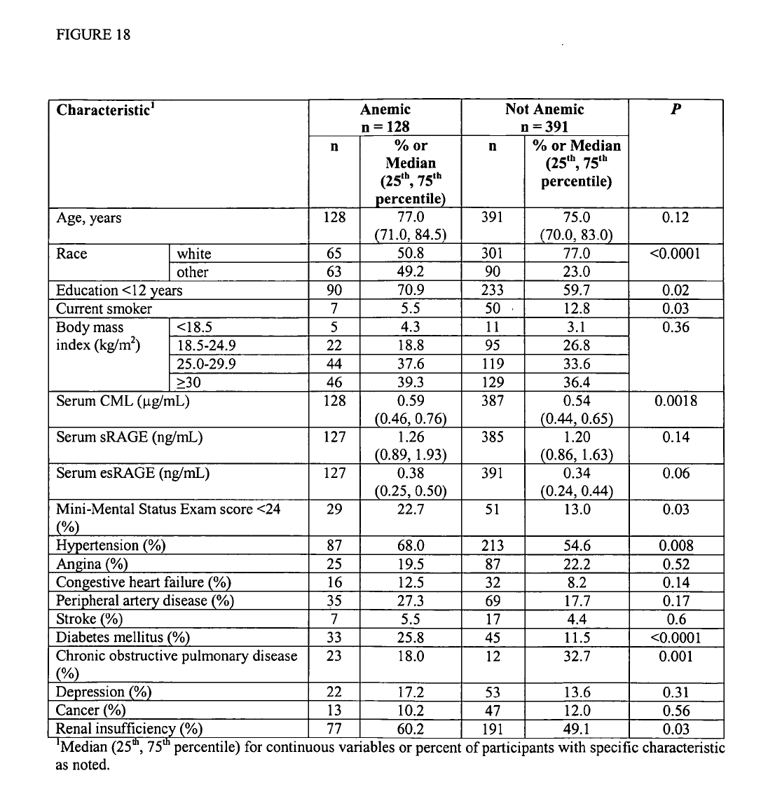

The demographic and health characteristics of 519 women with and without anemia are shown in the Table in FIG. 18. Of the 519 women, 128 (24.7%) had anemia. Women with anemia were more likely to be non-white, have a lower level of education, MMSE score <24, and to have hypertension, diabetes, and renal insufficiency, and less likely to be current smokers or to have chronic obstructive pulmonary disease. There were no significant differences in age, body mass index, angina, congestive heart failure, peripheral artery disease, stroke, depression, or cancer between women with and without anemia. Median serum CML concentrations were significantly higher in women with anemia compared to women without anemia, whereas median serum sRAGE and esRAGE levels were not significantly different between groups (P=0.14, P=0.06, respectively).

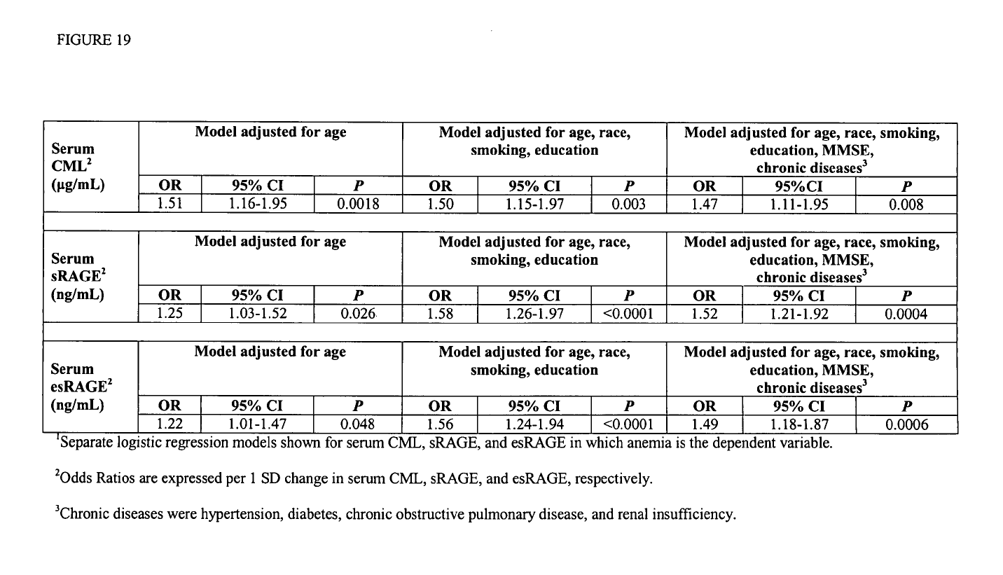

Separate multivariate logistic regression models were used first to examine the cross-sectional relationship between serum CML, sRAGE, and esRAGE with anemia (Table in FIG. 19). Serum CML, sRAGE, and esRAGE (per 1 S.D. increase), respectively, were associated with anemia in separate multivariate logistic regression models adjusting for age, for age, race, smoking, and education; and for age, race, smoking, education, MMSE score, hypertension, diabetes, chronic obstructive pulmonary disease, and renal insufficiency.

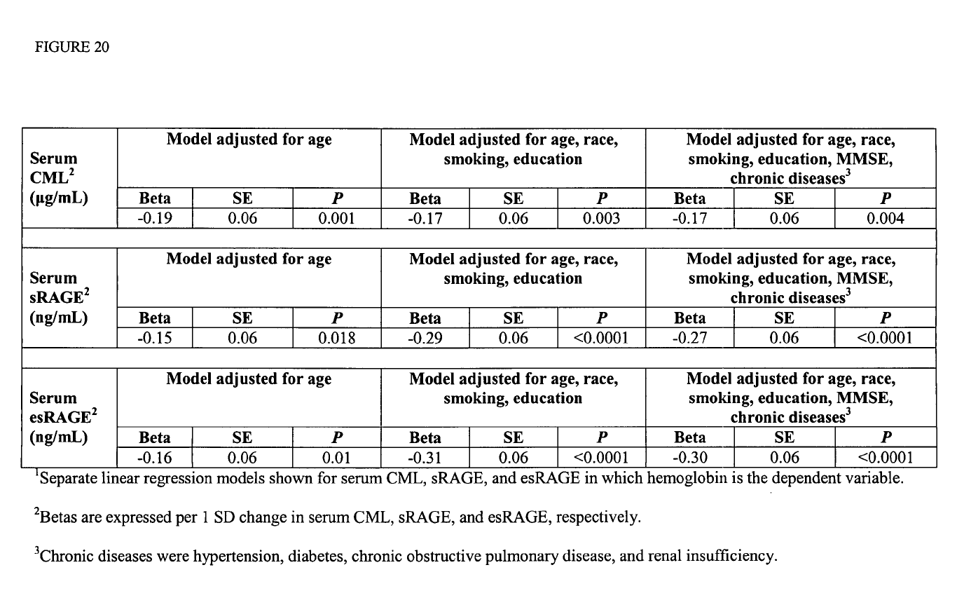

Serum CML, sRAGE, and esRAGE (per 1 S.D. increase), respectively, were inversely associated with hemoglobin in separate multivariate linear regression models adjusting for age, for age, race, smoking, and education; and for age, race, smoking, education, MMSE score, hypertension, diabetes, chronic obstructive pulmonary disease, and renal insufficiency (Table in FIG. 20).

In an additional set of analyses, all women who were diabetic were excluded. Serum CML, sRAGE, and esRAGE (per 1 S.D. increase), respectively, were associated with anemia in separate multivariate logistic regression models (O.R. 1.29, 95% C.I: 1.01-1.64, P=0.04; O.R. 1.47, 95% C.I. 1.14-1.91, P=0.003; O.R. 1.34, 95% C.I. 1.05-1.73, P=0.02), adjusting for age, race, smoking education, MMSE score, hypertension, chronic obstructive pulmonary disease, and renal insufficiency. Serum CML, sRAGE, and esRAGE (per 1 S.D. increase), respectively, were inversely associated with hemoglobin in separate multivariate linear regression models (beta=−0.19, S.E.=0.06, P=0.0018; beta=−0.29, S.E.=0.06, P<0.0001; beta=−0.26, S.E.=0.06, P<0.0001), adjusting for age, race, smoking education, MMSE score, hypertension, chronic obstructive pulmonary disease, and renal insufficiency.

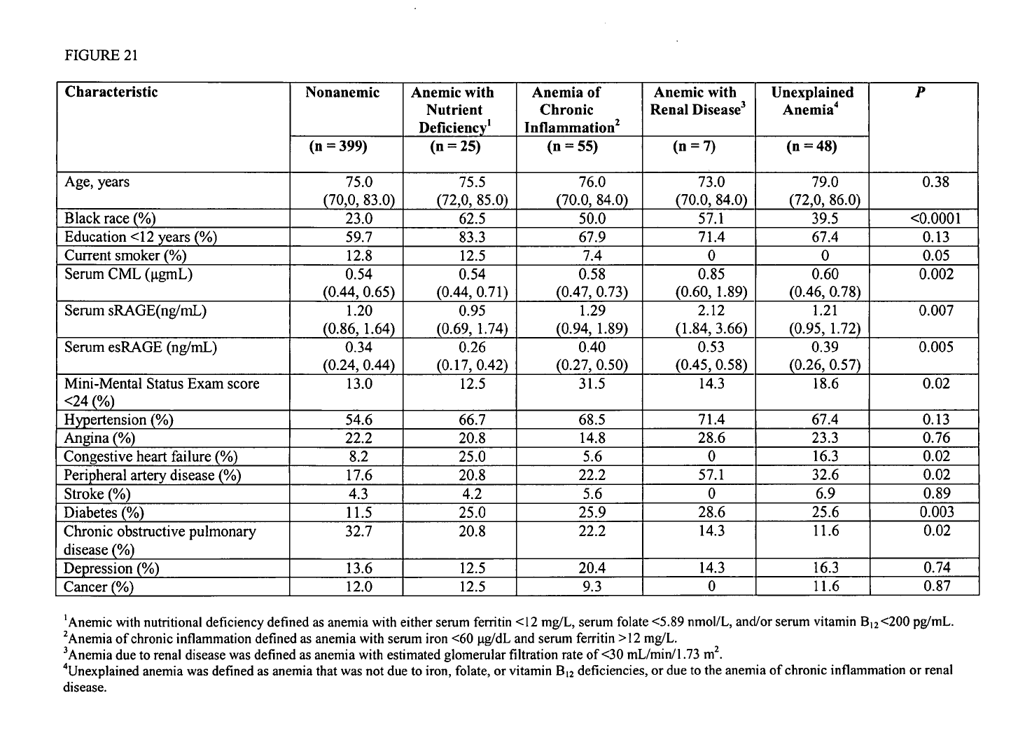

Median serum CML, sRAGE, and esRAGE concentrations in different types of anemia are shown in the Table shown in FIG. 21. Serum CML concentrations were highest in women with anemia with renal disease and unexplained anemia. Median serum sRAGE and esRAGE concentrations were highest in women with anemia of chronic inflammation, anemia with renal disease, and unexplained anemia.

The present study suggests that elevated AGEs, as indicated by serum CML, are inversely associated with hemoglobin and directly associated with anemia in older, community-dwelling women. To our knowledge, this is the first study to report an association between elevated AGEs and and anemia in a population of community-dwelling adults. The present study is consistent with a previously reported association of elevated AGEs and anemia among patients with type 2 diabetes, and it extends these findings, as the association between AGEs and anemia was also consistent among patients without diabetes. The present study also adds to what is known about AGEs and anemia by showing the relationship between elevated circulating RAGE and anemia. As noted previously, AGEs alter the deformability of erythrocytes and increase interactions between erythrocytes and the endothelial surface via interactions of erythrocyte AGE with RAGE. In addition, CML forms adducts with hemoglobin, but whether the formation of hemoglobin-CML affects the lifespan of erythrocytes or contributes to anemia via other biological mechanisms should be examined in future studies.

Serum CML, sRAGE, and esRAGE concentrations appear to be the highest in women with renal disease compared with women who had other types of anemia or were non-anemic. Elevated serum or plasma AGEs have previously been described in patients with diabetic nephropathy and end-stage renal disease, and elevated circulating RAGE has been described in patients with end-stage renal disease. AGEs can contribute to chronic kidney disease by inducing glomerulosclerosis and interstitial fibrosis. The progression of chronic kidney disease can contribute to elevated systemic levels of AGEs, thus worsening a vicious cycle.

In conclusion, elevated CML, a dominant AGE, is independently associated with anemia in older, moderately to severely disabled community-dwelling women. AGEs are a potential target for interventions to prevent onset as well as progression of anemia, as serum AGEs can be lowered by change in dietary pattern and pharmacological treatment.

### Example 6

### Elevated Serum Advanced Glycation End Products are Associated with Renal Insufficiency: The Baltimore Longitudinal Study of Aging

Chronic renal insufficiency affects more than fifteen million people in the United States and is associated with high cardiovascular disease morbidity and mortality. The factors that affect the progression of chronic renal insufficiency and increase the risk of cardiovascular disease have been incompletely characterized. Advanced glycation end products (AGEs) are bioactive molecules that are formed by the non-enzymatic glycation of proteins and other molecules, and AGEs are implicated in the pathogenesis of renal insufficiency, diabetes, and atherosclerosis.

We postulated that elevated levels of serum AGE were associated with chronic renal insufficiency and were predictive of new renal insufficiency in subjects with normal baseline renal function. To address this hypothesis, the relationship between CML, a dominant AGE, and renal insufficiency and estimated glomerular filtration rate in community-dwelling adults was examined.

Study Population

The study subjects consisted of participants in the Baltimore Longitudinal Study of Aging (BLSA) who were seen between April 2002 and August 2007. The BLSA is a prospective open cohort study of community-dwelling volunteers, largely from the Baltimore/Washington area. The study was established in 1958 and is described above and in detail elsewhere (Shock et al., Normal Human Aging: the Baltimore Longitudinal Study of Aging. Washington, D.C., U.S. Government Printing Office, 1984, incorporated herein by reference).

Laboratory Studies

Serum creatinine was measured using the Jaffe method. Blood samples were stored continuously at −70° C. until the time of analyses of serum AGEs. The measure of serum AGEs in this study was serum carboxymethyl-lysine (CML). CML is a dominant circulating AGE, the best characterized of all the AGEs, and a dominant AGE in tissue proteins. CML was measured using a competitive ELISA (AGE-CML ELISA, Microcoat, Penzberg, Germany) as described above. This assay has been validated, is specific, and shows no cross-reactivity with other compounds. Measurements were all performed in duplicate according to the protocol of the manufacturers, and the results were averaged. The within assay and between assay coefficients of variation (CVs) for serum CML were both <5%.

Statistical Analysis

Continuous variables were compared using Wilcoxon rank-sum test. Categorical variables were compared using chi-square tests. Body mass index (BMI) and renal insufficiency values are defined as above. Logistic regression models were used to examine the relation between serum CML and other factors with renal insufficiency. Linear regression models were used to examine the same cross-sectional relationships where the dependent variable was estimated glomerular filtration rate. Variables that were significantly associated with renal insufficiency and estimated glomerular filtration rate in univariate analyses were entered into multivariate logistic and linear regression models. In linear and logistic regression models, one standard deviation in concentration of serum CML was used as the unit of change. The statistical program used was SAS (SAS Institute, Cary, N.C.), with data analysis conducted by Kai Sun. The level of significance used in this study was P<0.05.

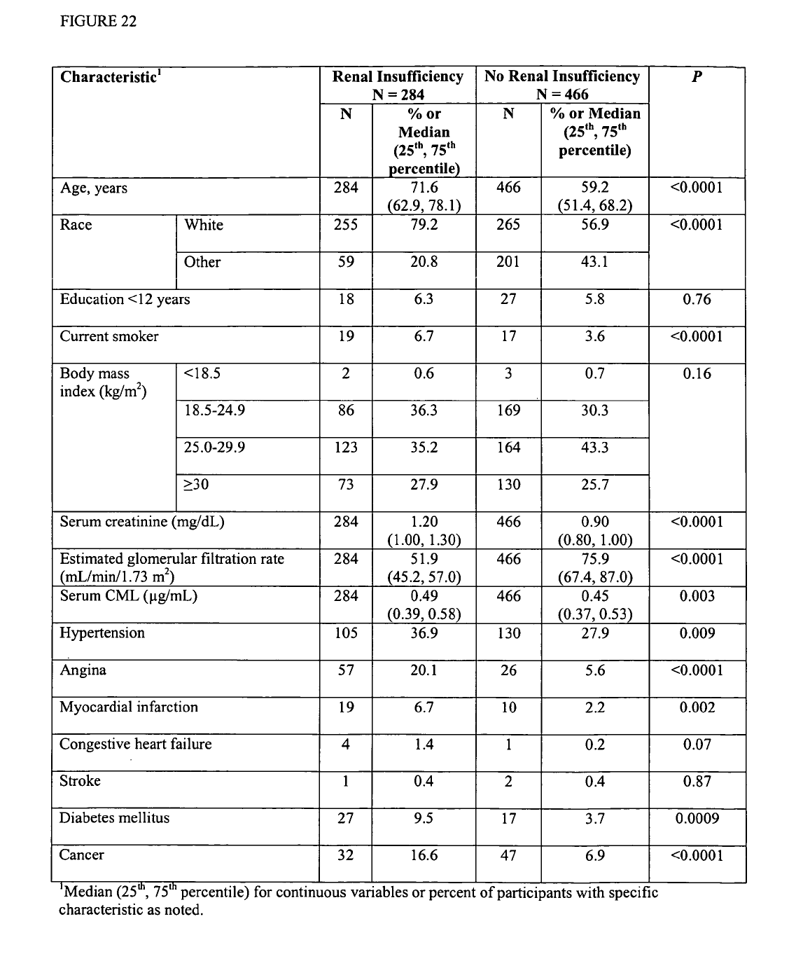

The demographic and health characteristics of 750 men and women with and without renal insufficiency are shown in the Table in FIG. 22. Overall, mean (SD) serum CML was 0.47 (0.13) μg/mL. Of 750 adults, 284 (37.9%) had renal insufficiency. Those with renal insufficiency were more likely to be older, white, current smokers, and to have hypertension, angina, a history of myocardial infarction, diabetes mellitus, and cancer. There were no significant differences in education, body mass index, or prevalence of heart failure, or stroke between those with and without renal insufficiency. The prevalence of heart failure and stroke were both less than 2% (Table in FIG. 22). The proportion of subjects with estimated glomerular filtration rate >90, 60-89, 30-59, 15-29, and <15 mL/min/1.73 m² was 13.1%, 49.2%, 36.4%, 2.1%, and 0.1%, respectively.

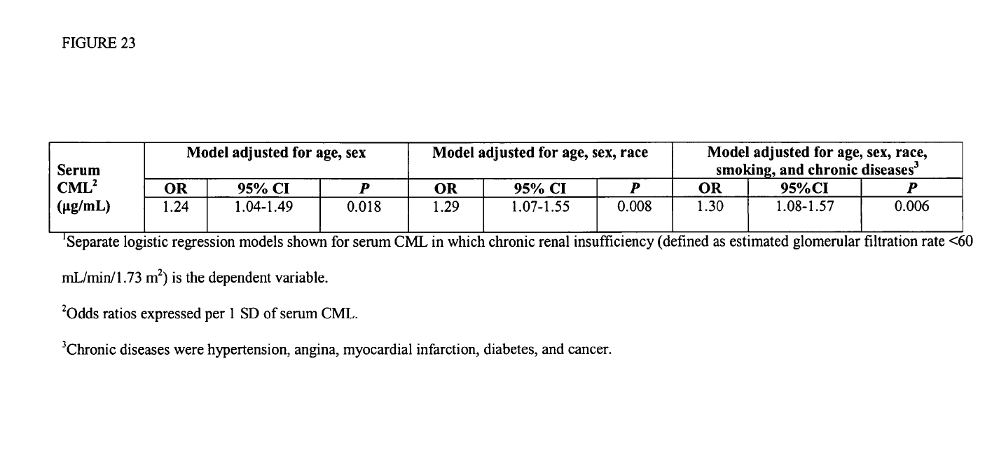

Separate multivariate logistic regression models were used to examine the cross-sectional relationship between serum CML and renal insufficiency (Table in FIG. 23). Serum CML was significantly associated with increased odds of prevalent renal insufficiency in models adjusted for age and sex, adjusted for age, sex, and race, and adjusted for age, sex, race, hypertension, angina, myocardial infarction, diabetes, and cancer (Table in FIG. 23).

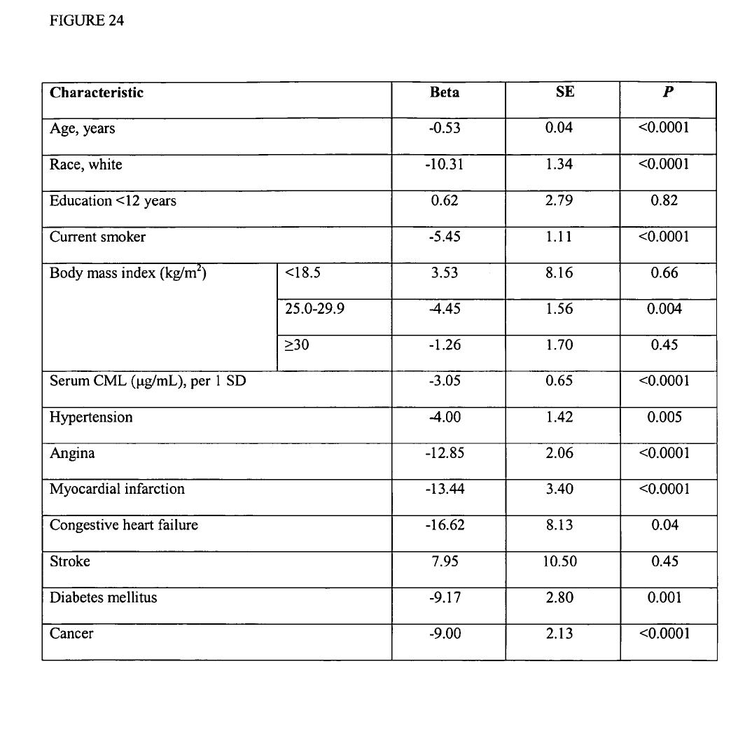

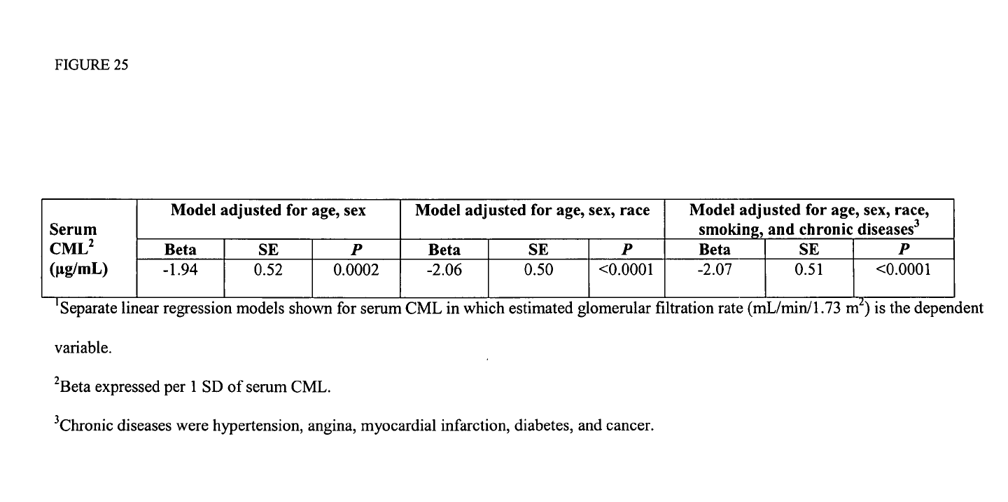

The relation between serum CML and estimated glomerular filtration rate was examined in univariate linear regression analyses shown in the Table in FIG. 24. Age, race, current smoking, overweight (BMI 25.0-29.9 kg/m2), hypertension, angina, myocardial infarction, heart failure, diabetes, and cancer were associated with estimated glomerular filtration rate. Education, stroke, and underweight, and obesity categories of BMI were not associated with estimated glomerular filtration rate. Serum CML was significantly associated with estimated glomerular filtration rate, in separate multivariate linear regression models adjusting for age and sex, adjusting for age, sex, and race, and adjusting for age, sex, race, smoking, and chronic diseases (Table in FIG. 25).

Taken together, these studies suggest that elevated serum CML is independently associated with renal insufficiency in community-dwelling men and women. Elevated serum or plasma AGEs have been described in patients with diabetic nephropathy and end-stage renal disease. To our knowledge, the present study is the first to show that elevated serum AGEs are associated with renal insufficiency in a relatively healthy cohort of community-dwelling men and women.

AGEs are metabolized and removed by the kidney, but the kidney is also a site for accumulation of AGEs and AGE-related damage. The serum concentrations of CML among adults with renal insufficiency in this study were lower than CML concentrations described adults with diabetic nephropathy and diabetics with retinopathy. AGEs have been implicated in the pathogenesis of diabetic nephropathy and complications of end-stage renal disease. AGEs upregulate inflammation and the synthesis of fibronectin, laminin, and collagen IV in the kidney and promote glomerular sclerosis, fibrosis, and hypertrophy. The kidney is affected by AGEs, and declining renal function entails an increase in serum AGEs, thereby amplifying damage from AGEs. AGEs are not merely a marker of renal insufficiency, as treatment with AGE inhibitors improves renal function, suggesting a direct role of AGEs in the pathogenesis of renal insufficiency. This is in contrast to what has been shown with hyperhomocysteinemia in kidney disease, where levels rise with declining renal function, but treatment has not been shown to be substantially beneficial.

The link between chronic kidney disease and cardiovascular disease has been largely attributed to endothelial dysfunction. An accumulation of AGEs in serum and tissues due to declining renal function may further exacerbate endothelial dysfunction and atherosclerosis. AGEs play a role in atherosclerosis by accumulating in arterial walls, increasing arterial stiffness by cross-linking collagen, contributing to the oxidation of low-density lipoprotein (LDL), cross-link with LDL and immunoglobulins in the subendothelium, initiating monocyte migration across endothelial cells, and upregulating inflammation via receptor for AGE (RAGE) and activation of transcription factor nuclear factor-KB.

In conclusion, elevated CML, a dominant AGE, is independently associated with renal insufficiency in community-dwelling men and women. AGES are a potential target for interventions to prevent onset as well as progression of renal insufficiency, as serum AGEs can be lowered by change in dietary pattern and pharmacological treatment.

### Example 7

### Advanced Glycation End Products and their Circulating Receptors and Level of Kidney Function in Older Community-Dwelling Women

Serum levels of AGE, sRAGE, and esRAGE were assayed in a group of older women to determine if a correlation between such levels and kidney function could be identified.

### Study Participants

A cross-sectional study was conducted among 559 women, aged 65 and older, from the Women's Health and Aging Studies (WHAS) I, representative of the one-third most disabled women residing in the community in Baltimore, Md. Recruitment and exclusion criteria are discussed in Example 1. Further details on the methods and sampling design of the WHAS studies are published elsewhere (Guralnik et al., The Women's Health and Aging Study: Health and Social Characteristics of Older Women with Disability. Bethesda, Md., National Institute on Aging. NIH Publication No. 95-4009, 1995).

Data Collection

Data collected on the subjects is described above. Standardized questionnaires were administered in the participant's home by trained interviewers including questions regarding race, current smoking status, and education. A physical examination was performed and upon authorization blood was drawn. The definitions for the chronic diseases reported in this study were adjudicated by WHAS co-investigators based on standardized algorithms that combined information from the questionnaire, physical examination, and physician contact (Guralnik et al., 1995). The Mini-Mental Status Examination (MMSE) was administered at enrollment (Folstein et al., J Psychiatr Res 12:189-198, 1975). Women were seen every 6 months for a follow-up visit for 36 months, and phlebotomy was repeated at the 12 and 24 month follow-up visits. Further details on the methods and sampling design of the WHAS studies are published elsewhere (Guralnik et al., 1995).

Laboratory Studies

There were 1002 women enrolled in the Women's Health and Aging Study I. Eight hundred seventy-nine women returned for the 12-month follow-up visit, of whom 580 participated in the blood drawing. The 559 women involved in the present study were significantly younger, and a higher proportion had MMSE score <24, level of education <12 years, and stroke compared with the 320 women who are not included in the present analysis. Analyses of serum AGEs, sRAGE, and esRAGE were done at the 12-month follow-up visit rather than at enrollment because of a greater availability of serum from this visit. Non-fasting blood samples were obtained by venipuncture between 9 AM and 2 PM. Processing, aliquoting, and freezing were carried out at the Core Genetics Laboratory of The Johns Hopkins University School of Medicine following a standardized protocol. Blood samples were delivered to Quest Diagnostics Laboratories (Teterboro, N.J.) and in part stored continuously at −70° C. until the time of analyses for serum AGEs and circulating RAGE.

The measure of serum AGEs in this studywas serum carboxymethyl-lysine (CML) as discussed in Example 1. CML was measured using a competitive ELISA (AGE-CML ELISA, Microcoat, Penzberg, Germany) as described above. Total sRAGE was measured using a sandwich ELISA (Quantikine Human RAGE Immunoassay, R & D Systems, Minneapolis, Minn.) as described above. This assay measures C-truncated RAGE that has been enzymatically cleaved from the cell surface as well as esRAGE. Serum esRAGE was measured using ELISA (B-Bridge International, Mountain View, Calif.). Measurements were all performed in duplicate according the protocol of the manufacturers, and the results were averaged.

Of 548 women, 283 (51.6%) had reduced GFR at baseline. Serum CML was associated with reduced GFR (Odds Ratios [O.R.; all expressed per 1 Standard Deviation], 1.98, 95% Confidence Interval [C.I.] 1.41-2.76, P<0.001) in a multivariate logistic regression model adjusting for age, race, hemoglobin A_{1c}, and chronic diseases. Serum sRAGE (ng/mL) and esRAGE (ng/mL), respectively, were associated with reduced GFR (O.R. 1.42, 95% C.I. 1.12-1.79, P=0.003; O.R. 1.42, 95% C.I. 1.14-1.77, P=0.001) in separate multivariate logistic regression models, adjusting for potential confounders. Of 230 women without reduced GFR at baseline, 32 (13.9%) developed reduced GFR by the follow-up visit 12 months later. Serum CML (μg/mL), sRAGE (ng/mL), and esRAGE (ng/mL), respectively, at baseline was associated with the prevalence of reduced GFR 12 months later (O.R. 1.80, 95% C.I. 1.19-2.71, P=0.005; O.R. 1.32, 95% C.I. 1.01-1.74, P=0.05; O.R. 1.33, 95% C.I. 1.01-1.77, P=0.05) in separate multivariate logistic regression models adjusting for potential confounders.

Statistical Analysis

Continuous variables were compared using Wilcoxon rank-sum test. BMI, MMSE, and GFR were characterized as described above in Example 1. Logistic regression models were used to examine separately the relationships of serum CML, sRAGE, and esRAGE, with prevalent reduced GFR at baseline and prevalent reduced GFR 12 months later, excluding prevalent cases of reduced GFR at baseline. Linear regression models were used to examine the same cross-sectional relationships where the dependent variable was estimated GFR at baseline. Variables that were significant in the univariate analyses were entered into the multivariate logistic regression models and multivariate linear regression models. Diabetes was added in alternative multivariate models because of the known strong relationship between diabetes and chronic kidney disease. In linear and logistic regression models, a one standard deviation in concentration of serum CML, sRAGE, and esRAGE, respectively, was used as the unit of change. Spearman correlation was used for examining correlation between serum CML, esRAGE, and sRAGE. The statistical program used was SAS (SAS Institute, Cary, N.C.), with data analysis conducted by Kai Sun. The level of significance used in this study was P<0.05.

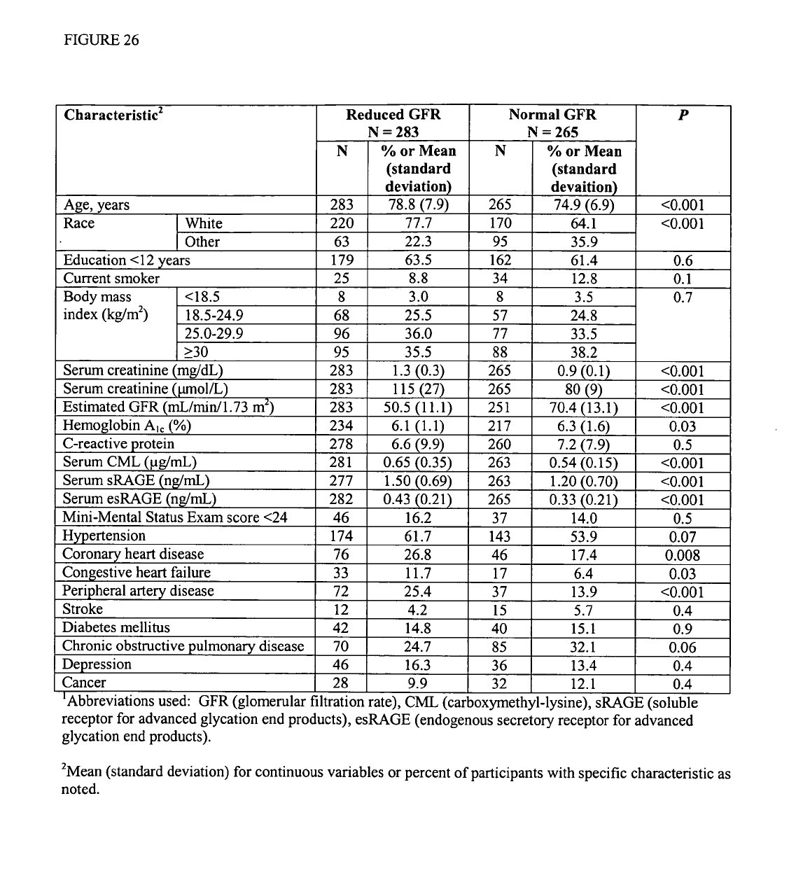

The demographic and health characteristics of 548 women with and without reduced GFR are shown in FIG. 26. Overall, mean (SD) serum creatinine at baseline was 1.1 (0.3) mg/dL, or 97 (27) umol/L, and mean (SD) estimated GFR was 60.1 (16.2) mL/min/1.73 m². Women with reduced GFR were more likely to be older, non-white, and to have coronary artery disease, congestive heart failure, and peripheral artery disease. There were no significant differences in education, current smoking, body mass index, cognitive function, or prevalence of hypertension, diabetes, stroke, chronic obstructive pulmonary disease, depression, or cancer between women with and without reduced GFR. Median serum CML, sRAGE, and esRAGE concentrations were significantly higher in women with reduced GFR compared with women without reduced GFR.

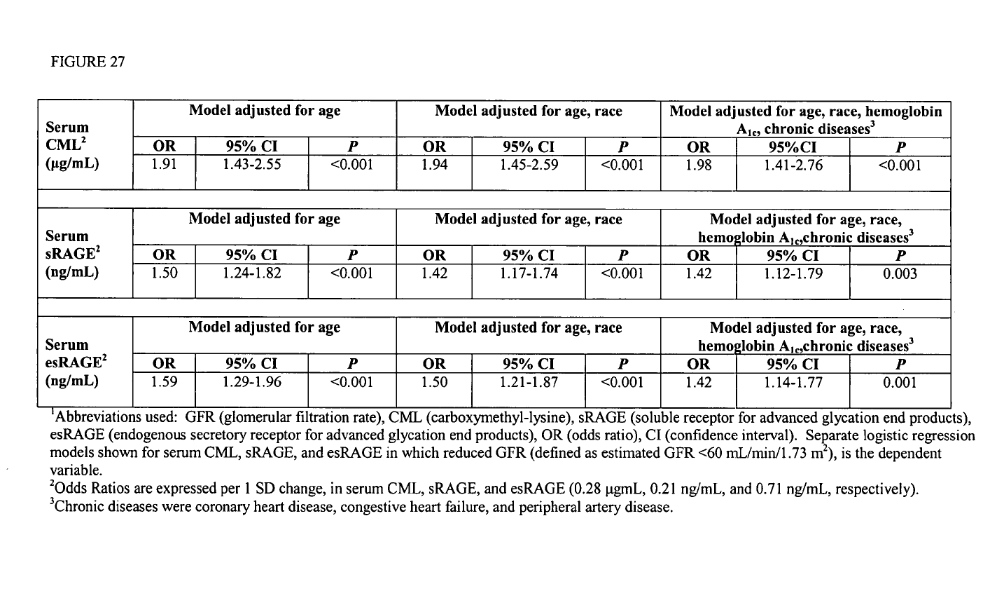

Separate multivariate logistic regression models were used first to examine the cross-sectional relationship between serum CML, sRAGE, and esRAGE with reduced GFR (FIG. 27). In models adjusted for age, and adjusted additional for race, hemoglobin A_{1c}, and coronary heart disease, congestive heart failure, and peripheral artery disease, serum CML, sRAGE, and esRAGE were all significantly associated with increased odds of prevalent reduced GFR (FIG. 27). Diabetes was not significantly associated with prevalent reduced GFR in the univariate analyses, but alternative models were run in which diabetes was added to a multivariate model as in Table 2 that included age, race, hemoglobin A_{1c}, and chronic diseases. Serum CML, sRAGE, and esRAGE (per 1 Standard Deviation [S.D.] increase) were associated with reduced GFR when diabetes was added to the respective multivariate models: O.R. 1.98, 95% C.I. 1.42-2.77, P<0.001; O.R. 1.42, 95% C.I. 1.14-1.76, P=0.002; O.R. 1.41, 95% C.I. 1.12-1.78, P<0.001.

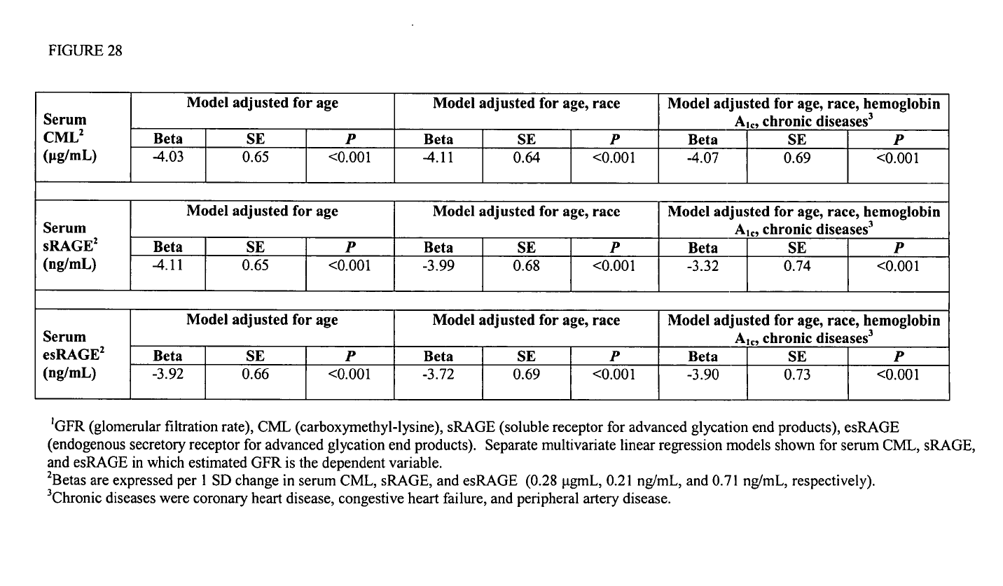

Serum CML, sRAGE, and esRAGE (per 1 S.D. increase), respectively, were associated with estimated GFR at baseline in separate linear regression models adjusting for age, and additionally adjusting for race, hemoglobin A_{1c} coronary heart disease, congestive heart failure, and peripheral artery disease (FIG. 28). Alternative models for serum CML, sRAGE, and esRAGE (per 1 S.D. increase), respectively, were also considered in which diabetes was added to the model, in addition to age, race, hemoglobin A_{1c}, coronary heart disease, congestive heart failure, and peripheral artery disease: beta=−4.10, SE=0.68, P<0.001; beta=−3.84, SE=0.73, P<0.001; beta=−3.25, SE=0.74, P<0.001, respectively.

Of the 548 women seen at baseline, 376 women were seen in follow-up 12 months later. Of 230 women without reduced GFR at baseline, 32 (13.9%) women developed reduced GFR by the follow-up visit 12 months later. Serum CML (μg/mL) at baseline, per 1 S.D. increase, was associated with prevalence of reduced GFR at 12 months (O.R. 1.80, 95% C.I. 1.19-2.71, P=0.005) in a multivariate logistic regression model adjusting for age, race, hemoglobin A_{1c}, coronary heart disease, congestive heart failure, and peripheral artery disease. Adding diabetes to the previous model yielded similar results (O.R. 1.80, 95% C.I. 1.19-2.71, P=0.005). Serum sRAGE (ng/mL) at baseline, per 1 S.D. increase, was associated with prevalence of reduced GFR at 12 months (O.R. 1.32, 95% C.I. 1.01-1.74, P=0.05). Adding diabetes to the previous model yielded similar results (O.R. 1.32, 95% C.I. 1.01-1.74, P=0.04). Serum esRAGE (ng/mL) at baseline, per 1 S.D. increase, was associated with prevalence of reduced GFR at 12 months (O.R. 1.33, 95% C.I. 1.01-1.77, P=0.05) in a multivariate logistic regression model adjusting for age, race, coronary heart disease, congestive heart failure, and peripheral artery disease. Adding diabetes to the previous model yielded similar results (O.R. 1.33, 95% C.I. 1.01-1.77, P=0.04).

At baseline, among 82 women with diabetes and 466 women without diabetes, mean (SD) serum CML, sRAGE, and esRAGE concentrations were, respectively, 0.55 (0.2) and 0.61 μg/mL (P=0.08), 1.35 (0.79) and 1.35 (0.70) ng/mL (P=0.9), and 0.37 (0.24) and 0.38 (0.21) ng/mL (P=0.7).

This study shows that elevated serum CML and circulating RAGE are associated with reduced GFR in older community-dwelling women and suggests that these associations are independent of the multiple morbidities present in this high-risk, disabled population. Elevated circulating AGEs have been described in diabetes and in chronic kidney disease with or without diabetes. Patients with chronic kidney disease and end-stage renal disease² were found to have elevated RAGE expression and circulating RAGE, respectively. RAGE mRNA is increased in peripheral mononuclear cells obtained from patients with chronic kidney disease. Increased levels of RAGE may be a protective mechanism against the pro-inflammatory effect of circulating AGE on cells. The present study shows that elevated serum AGEs and circulating RAGE are associated with reduced GFR in a population-based study of community-dwelling adults. The present study also suggests that elevated serum AGEs and circulating RAGE are predictive of the development of reduced GFR.

### Example 8

### Serum Carboxymethyl-Lysine, an Advanced Glycation End Product, is Associated with Increased Aortic Pulse Wave Velocity in Adults

The relationship between advanced glycation end products and arterial stiffness has previously been examined in highly selected groups of patients with diabetes or hypertension. Our aim was to determine whether elevated serum advanced glycation end products are associated with increased arterial stiffness in relatively healthy, community-dwelling adults.

Study Population

The study subjects consisted of participants in the Baltimore Longitudinal Study of Aging (BLSA) who were seen between April 2002 and August 2007. The BLSA is a prospective open cohort study of community-dwelling volunteers, largely from the Baltimore/Washington area. The study was established in 1958 and is described above and in detail elsewhere (Shock et al., Normal Human Aging: the Baltimore Longitudinal Study of Aging. Washington, D.C., U.S. Government Printing Office, 1984, incorporated herein by reference).

Laboratory Studies

Blood samples were drawn from the antecubital vein between 7 and 8 AM after an overnight fast. Subjects were not allowed to smoke, engage in physical activity, or take medications before the sample was collected. Concentrations of plasma triglycerides and total cholesterol were determined by an enzymatic method (Abbott Laboratories ABA-200 ATC Biochromatic Analyzer, Irving, Tex.). The concentration of high-density lipoprotein cholesterol was determined by a dextran sulfate-magnesium precipitation procedure. Low density lipoprotein cholesterol concentrations were estimated by using the Friedewald formula. The fasting plasma glucose concentration was measured by the glucose oxidase method (Beckman Instruments, Inc., Fullerton, Calif.).

Blood samples were stored continuously at −70° C. until the time of analyses of serum AGEs. The measure of serum AGEs in this study was serum carboxymethyl-lysine (CML). CML is a dominant circulating AGE, the best characterized of all the AGEs, and a dominant AGE in tissue proteins. CML was measured using a competitive ELISA (AGE-CML ELISA, Microcoat, Penzberg, Germany) as described above. This assay has been validated, is specific, and sho. ws no cross-reactivity with other compounds. Measurements were all performed in duplicate according to the protocol of the manufacturers, and the results were averaged. The within assay and between assay coefficients of variation (CVs) for serum CML were both <5%.

Statistical Analysis

Continuous variables were compared using Wilcoxon rank-sum test. Categorical variables were compared using chi-square tests. Body mass index (BMI) values are defined as above. Serum CML values was normally distributed. Univariate and multivariate linear regression models were used to examine the relationship between demographic, anthropometric, laboratory, and clinical characteristics and PWV. Age and body mass index were used as categorical variables in the analyses because the relationships between age and body mass index, respectively, with PWV were not linear. Variables were included in the multivariate model if they were significant in the univariate analyses, except for cardiovascular diseases. All analyses were conducted using SAS version 9.13 (SAS, Cary, N.C.).

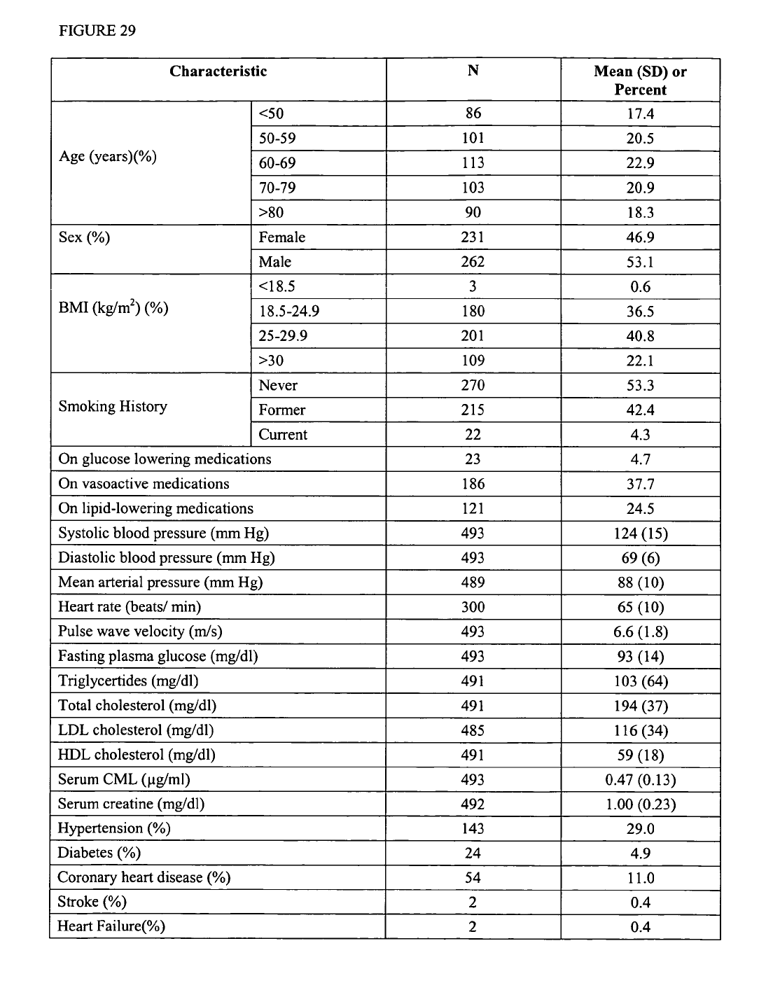

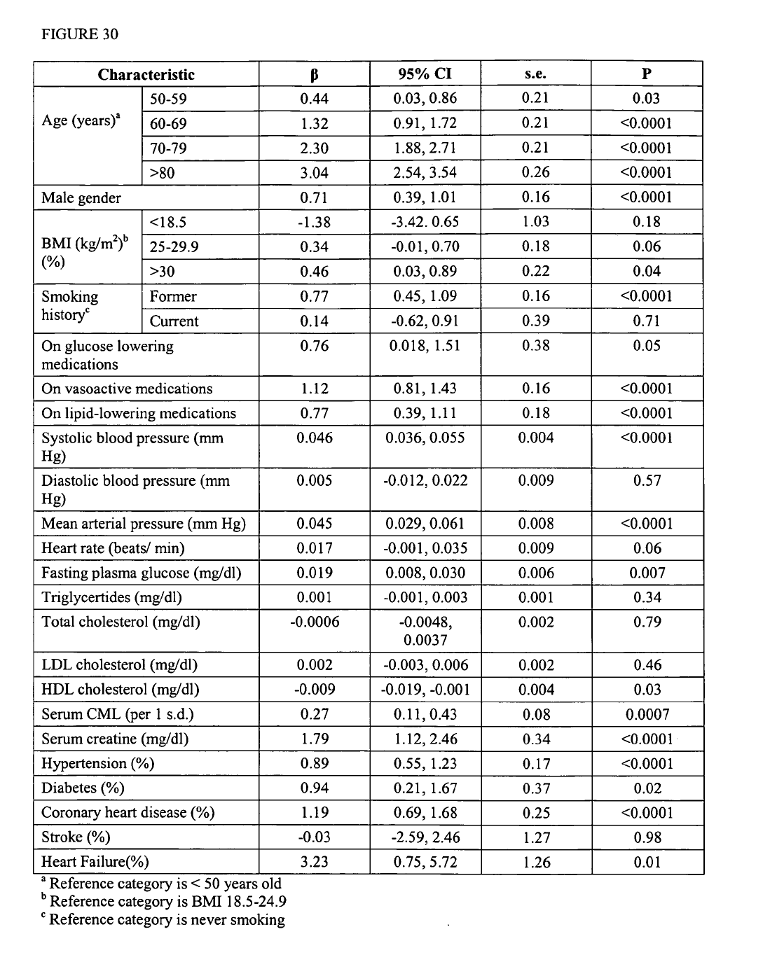

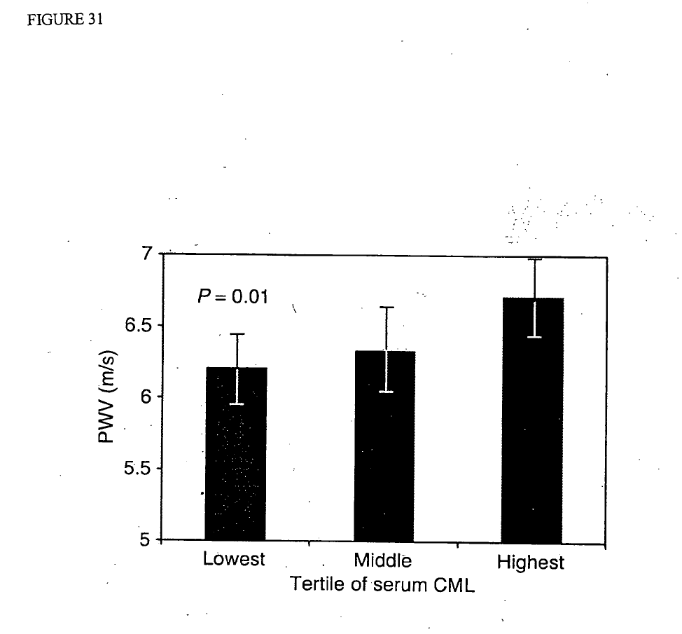

Results Demographic, disease, and other characteristics of the 493 study subjects are shown in FIG. 29. Overall, mean (s.d.) PWV was 6.6±1.8 m/s. Among adults aged <50, 50-59, 60-69, 70-79, and ≧80 years, mean (s.d.) PWV was 5.3±1.1, 5.8±1.2, 6.7±1.5, 7.6±1.7, and 8.4±1.8, respectively (P<0.0001). The relationships between demographic, disease, serum CML, and other factors with aortic PWV are shown in FIG. 30. Age, gender, body mass index, former smoking, mean arterial pressure, fasting plasma glucose, high-density lipoprotein cholesterol, serum CML, serum creatinine, use of glucose-lowering, vasoactive, and lipid-lowering drug(s), hypertension, diabetes, coronary heart disease, and heart failure were associated with aortic PWV in univariate linear regression models. Diastolic blood pressure, triglycerides, total cholesterol, low-density lipoprotein cholesterol, and stroke were not significantly associated with aortic PWV wave velocity. Heart rate measurements were only available in 300 of the participants, and heart rate was marginally associated with aortic PWV (P=0.06). Serum CML was divided into tertiles, with tertile cutoffs at 0.41 and 0.52 μg/ml. Geometric mean aortic PWV increased across tertiles of serum CML (P=0.01, by ANOVA), as shown in FIG. 31.

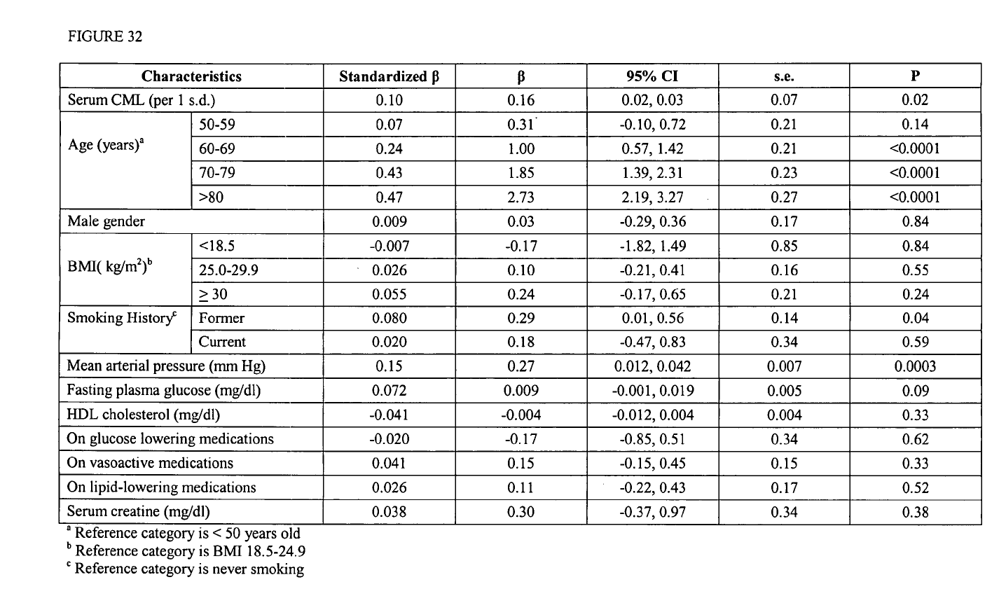

Serum CML, per 1 s.d. was associated with aortic PWV, after adjusting for age, gender, body mass index, and smoking, mean arterial pressure, fasting plasma glucose, high-density lipoprotein cholesterol, and serum creatinine as shown in FIG. 32. There was a weak association of PWV with heart rate (r=0.11, P=0.06). When heart rate was added to the same model as in FIG. 32, the number of subjects in the model was only 300 due to the more limited number of measurements of heart rate. In the multivariate model that included heart rate, serum CML, per 1 s.d, was associated with aortic PWV (β=0.16, s.e.=0.08, P=0.049).

There was no significant interaction term between serum CML and fasting plasma glucose. In an alternative model that included diabetes in addition to all the variables as in the multivariate model in FIG. 32, serum CML (per 1 s.d.) was associated with aortic PWV (β=0.16, s.e.=0.07, P=0.03). After excluding all patients with diabetes, mean (s.d) CML was 0.47 (0.13) μg/ml. After excluding all patients with diabetes, serum CML (per 1 s.d.) was associated with aortic PWV (β=0.18, s.e.=0.07, P=0.009) in a model that included all the variables as in FIG. 32.

The present study shows that in community-dwelling adults, elevated serum AGEs are independently associated with increased arterial stiffness, as indexed by increased aortic PWV. To our knowledge, this is the first study to show that serum AGEs are associated with arterial stiffness in a cohort of relatively healthy, community-dwelling adults. Serum AGEs were also associated with arterial stiffness even after excluding all patients with diabetes.

The present study suggests that AGEs may be a major risk factor for arterial stiffness. It should be noted that the values of PWV in our study may be lower than those reported in other studies because in our measurement of the distance traveled by the pulse wave, we subtracted the difference between the manubrium and the carotid sampling site from the sum of the distances between the manubrium and the umbilicus and the umbilicus and the femoral sampling sites. This correction was done to account for the fact that the centrifugal travel of the pulse wave occurs simultaneously in the aortic arch/carotid segment and the aortic arch/ascending aorta segment. However, other investigators have used different strategies for measuring the distance traveled by the pulse wave. An expert consensus panel commented that the various methods are approximations, and to date, none has emerged as preferred over the others (Laurent et al., Eur Heart J 2006; 27:2588-2605).

### Example 9

### Carboxymethyl-Lysine, an Advanced Glycation End Product, and Decline of Renal Function in Older Community-Dwelling Adults in Italy

Plasma levels of AGE were assayed in a population of Italian subjects to determine if a correlation between such levels and kidney function could be identified.

Study Population

The participants consisted of men and women, aged 65 and older, who participated in the Invecchiare in Chianti, “Aging in the Chianti Area” (InCHIANTI) study, a population-based study conducted in two small towns, Greve in Chianti and Bagno a Ripoli, in Tuscany, Italy. The rationale, design, and data collection have been described elsewhere, and the main outcome of this longitudinal study is mobility disability (Ferrucci et al., J Am Geriatr Soc 48:1618-1625, 2000, incorporated herein by reference).

Participants were enrolled after written, informed consent. The study protocol complied with the Declaration of Helsinki and was approved by the Italian National Institute of Research and Care on Aging Ethical Committee. The plan for secondary data analysis was approved by the Institutional Review Board of the Johns Hopkins University School of Medicine. Demographic information and information on smoking and medication use were collected using standardized questionnaires. Smoking history was determined from self-report and dichotomized in the analysis as “current smoking” versus “ever smoked” and “never smoked.” Education was recorded as years of school. All participants were examined by a trained geriatrician, and diseases were ascertained according to standard, pre-established criteria and algorithms based upon those used in the Women's Health and Aging Study for coronary heart disease, chronic heart failure, stroke, and cancer (Guralnik et al., National Institute on Aging, Bethesda. NIH publication no. 95-4009, 1995.

Laboratory Studies

Body mass index and MMSE were defined as above. Chronic kidney disease was defined as estimated glomerular filtration rate of <60 ml/min/1.73 m² using the four-variable Modification of Diet as described above. Participants were evaluated again for a three-year follow-up visit from 2001-2003 (n=926) and six-year follow-up visit from 2004-2006 (n=844). Vital status was determined using data from the Mortality General Registry maintained by the Tuscany Region.

Blood samples were collected in the morning after a 12-h fast. Aliquots of plasma and serum were immediately obtained and stored at −80° C. The measure of plasma AGEs in this study was plasma carboxymethyl-lysine (CML), one of the better characterized AGEs that is found in the circulation and in high concentrations in tissue proteins. CML was measured at enrollment using a competitive ELISA (AGE-CML ELISA, Microcoat, Penzberg, Germany) as described above. Variables are reported as medians (25th, 75^{th} percentiles) or as percentages.

Statistical Analysis

Characteristics of subjects according whether or not they had chronic kidney disease were compared using Wilcoxon rank sum tests for continuous variables and chi-square tests for categorical variables. Age and body mass index were analyzed as categorical variables because the relationship between age, body mass index, and renal function was not linear. Univariate and multivariate logistic regression models were used to examine the relationship between plasma CML and chronic kidney disease. Variables that were significant in the univariate analyses were entered into the multivariate analyses. Univariate and multivariate linear regression models were used to examine the relationship between plasma CML and eGFR. Cox proportional hazards models were used to examine the relationship between plasma CML at enrollment and the categorical outcomes of incident chronic kidney disease and all-cause mortality. The statistical program used was SAS (SAS Institute, Cary, N.C.), with data analysis conducted by Kai Sun. The level of significance used in this study was P<0.05.

Results

Of the 1,155 participants ‡65 years, seen at enrollment, 1,055 (91.3%) participated in the blood drawing. There were 1,012 (87.6%) participants who had plasma CML measurements available for this analysis at enrollment. The subjects who did not participate in the blood drawing were generally older and had greater comorbidity than the subjects who participated in the blood drawing, as reported elsewhere. Of the 1,012 participants seen at enrollment, 1,008 had both plasma CML and eGFR measurements available at the enrollment visit. Of the 1,008 subjects, 735 (72.9%) had eGFR measurements available at the three-year follow-up visit, and 643 (63.8%) had eGFR measurements available at the six-year follow-up visit. Of the 1,012 subjects with CML measurements at enrollment, 96 died between enrollment and the three-year follow-up visit, and 130 died between the three-year and six-year follow-up visit, 73 refused participation in the three-year follow-up visit, 34 refused participation in the six-year follow-up visit, and 20 moved out of the study area.

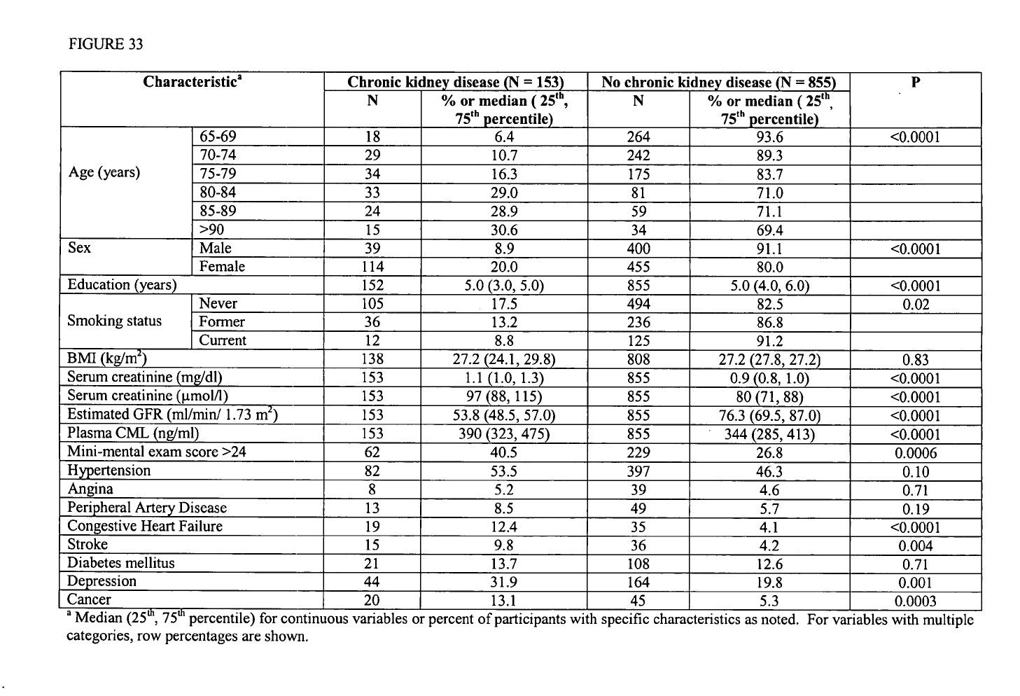

The demographic and health characteristics of 1,008 adults with and without chronic kidney disease at enrollment are shown in FIG. 33. Overall, mean (SD) serum CML was 365 (110) ng/ml. Of 1,008 adults, 153 (15.2%) had chronic kidney disease. Those with chronic kidney disease were more likely to be older, female, non-smokers, and to have lower level of education, MMSE<24, congestive heart failure, stroke, depression, and cancer. There were no significant differences in body mass index or prevalence of hypertension, angina, peripheral artery disease, or diabetes mellitus between those with and without chronic kidney disease. The proportion of subjects with estimated glomerular filtration rate ‡90, 60-89, 30-59, 15-29, and <15 ml/min/1.73 m2 was 17.9, 67.4, 14.8, 0.4, and 0.1%, respectively.

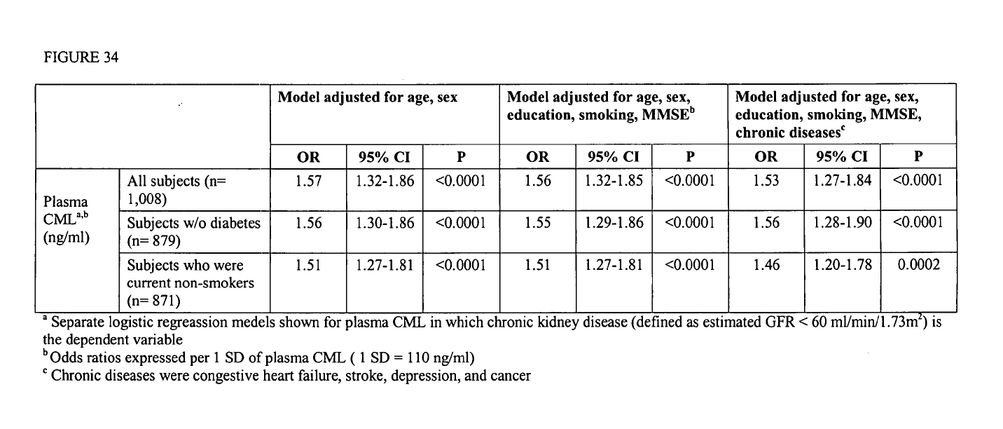

Separate multivariate logistic regression models were used to examine the cross-sectional relationship between plasma CML and chronic kidney disease at enrollment (FIG. 34). Plasma CML was significantly associated with increased odds of chronic kidney disease in models adjusting for age and sex, and additionally for education, smoking, and MMSE, and for chronic diseases (FIG. 34). After exclusion of participants who had diabetes, plasma CML was significantly associated with increased odds of chronic kidney disease in models adjusting for age and sex, and additionally for education, smoking, and MMSE, and for chronic diseases (FIG. 34). After exclusion of participants who were current smokers, plasma CML was significantly associated with increased odds of chronic kidney disease in models adjusting for age and sex, and additionally for education and MMSE, and for chronic diseases (FIG. 34).

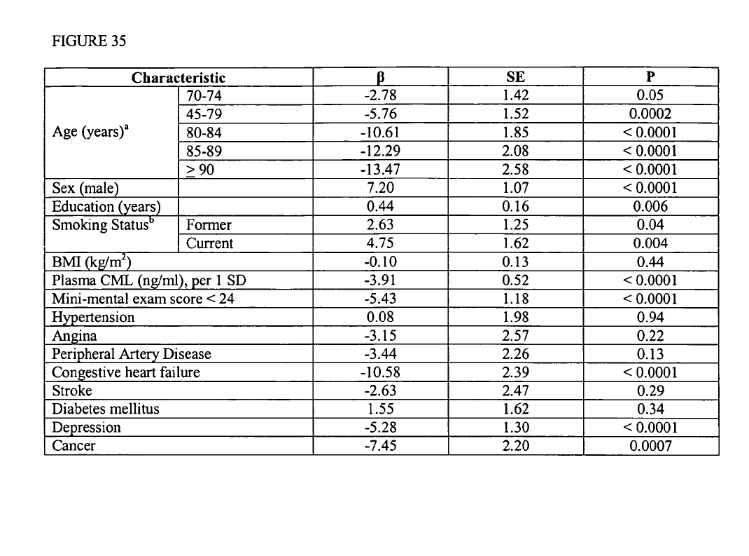

The cross-sectional relationship between plasma CML and eGFR at enrollment was examined in univariate linear regression analyses shown in FIG. 35. Older age, sex, education, smoking, plasma CML, MMSE<24, congestive heart failure, depression, and cancer were associated with eGFR. Body mass index, hypertension, angina, peripheral artery disease, stroke, and diabetes mellitus were not associated with eGFR.

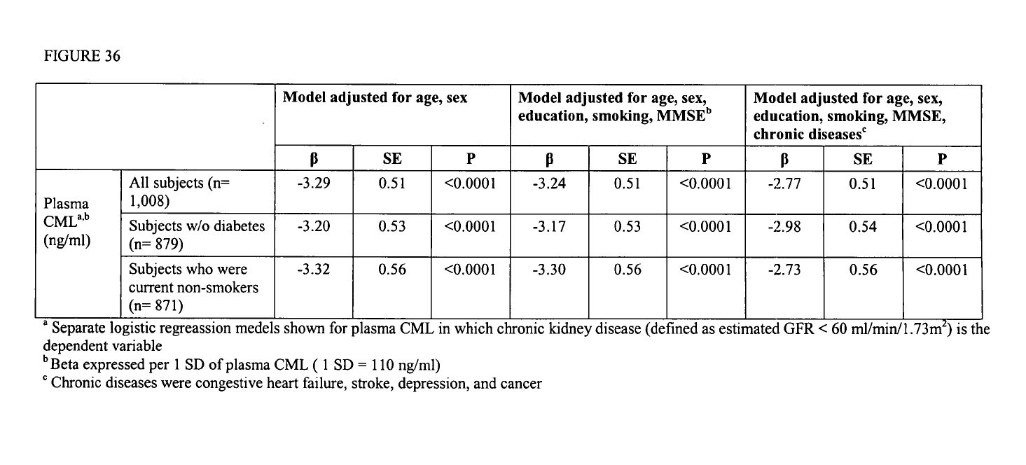

Separate multivariate linear regression models were used to examine the cross-sectional relationship between plasma CML and eGFR at enrollment (FIG. 36). Plasma CML was significantly associated with eGFR in separate models adjusting for age and sex, and additionally for education, smoking, and MMSE, and for chronic diseases. After exclusion of participants who had diabetes, plasma CML was significantly associated with eGFR in separate models adjusting for age and sex, and additionally for education, smoking, and MMSE, and for chronic diseases (FIG. 36). After exclusion of participants who were current smokers, plasma CML was significantly associated with eGFR in separate models adjusting for age and sex, and additionally for education and MMSE, and for chronic diseases (FIG. 36).

Of 855 participants who did not have chronic kidney disease at enrollment, 170 (19.9%) developed chronic kidney disease during the 6 years of followup. In multivariate Cox proportional hazards models, plasma CML (per 1 S.D.), was associated with incident chronic kidney disease, adjusting for age and sex (hazards ratio [H.R.] 1.15, 95% Confidence Interval [C.I.] 0.97-1.35, P=0.10), adjusting additionally for education, smoking, and MMSE (H.R. 1.15, 95% C.I. 0.97-1.36, P=0.10), and adjusting for the previous covariates and congestive heart failure, stroke, depression, and cancer (H.R. 1.15, 95% C.I. 0.97-1.36, P=0.10). After excluding participants with diabetes, of the 747 non-diabetic participants who did not have chronic kidney disease at enrollment, 140 (18.7%) developed chronic kidney disease during follow-up.

In multivariate Cox proportional hazards models, plasma CML (per 1 S.D.), was associated with incident chronic kidney disease, adjusting for age and sex (Hazards Ratio [H.R.] 1.17, 95% Confidence Interval [C.I.] 0.97-1.40, P=0.09), adjusting additionally for education, smoking, and MMSE (H.R. 1.16, 95% C.I. 0.97-1.40, P=0.10), and adjusting for the previous covariates and congestive heart failure, stroke, depression, and cancer (H.R. 1.18, 95% C.I. 0.98-1.42, P=0.07).

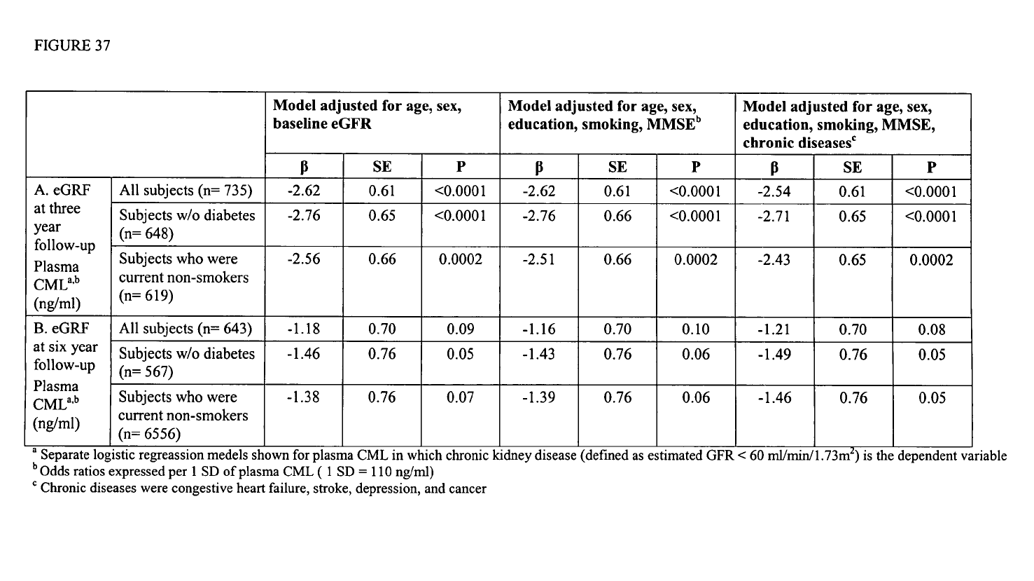

Of 855 participants who did not have chronic kidney disease at enrollment, 171 (20.0%) died during six years of follow-up. There was a strong competing risk of mortality during follow-up. Participants in the highest quartile of plasma CML compared to the lower three quartiles had higher all-cause mortality, adjusting for age and sex (H.R. 1.36, 95% C.I. 1.00-1.86, P=0.05), adjusting additionally for education, smoking, and MMSE (H.R. 1.38, 95% C.I. 1.01-1.88, P=0.04) and adjusting for the previous covariates and congestive heart failure, stroke, depression, and cancer (H.R. 1.44, 95% C.I. 1.03-2.02, P=0.03). There were 735 participants who had at least one or more eGFR measurements available from the six years of follow-up. The relationship between plasma CML at enrollment and eGFR at 3- and 6-year followup visits was examined in separate multivariate linear regression models (FIG. 37). Plasma CML was associated with eGFR at 3- and 6-year follow-up visits in models adjusting for age, sex, baseline eGFR, education, smoking, MMSE, and chronic diseases. After excluding participants with diabetes, plasma CML was associated with eGFR at 3- and 6-year follow-up visits in models adjusting for age, sex, baseline eGFR, education, smoking, MMSE, and chronic diseases (FIG. 37). After excluding participants who were current smokers, plasma CML was associated with eGFR at 3- and 6-year follow-up visits in models adjusting for age, sex, baseline eGFR, education, smoking, MMSE, and chronic diseases (FIG. 37). The relationships were generally stronger between plasma.

The present study shows that elevated plasma CML is independently associated with chronic kidney disease and eGFR in older adults living in the community. Elevated CML at baseline was an independent predictor of eGFR at 3 and 6 years' follow-up. To our knowledge, this is the first study to show that elevated circulating AGEs are an independent predictor of renal function in population-based study of communitydwelling men and women. Hyperglycemia is considered to increase the generation of endogenous AGEs, and the relationship between AGEs and renal disease has been studied extensively in patients with diabetes.

Another important new observation in the present study was that plasma CML was strongly associated with chronic kidney disease, eGFR, and eGFR at followup, even after excluding participants who had diabetes. These findings suggest that the potential adverse effects of AGEs on the kidney are applicable to the general population of older community-dwelling adults.

In conclusion, elevated plasma CML was associated with chronic kidney disease and reduced renal function, and elevated plasma CML was an independent predictor of renal function. The relationships between elevated plasma CML and reduced renal function were strong in older community dwelling men and women without diabetes.

### Example 10

### Plasma Carboxymethyl-Lysine, an Advanced Glycation End Product, and all-Cause and Cardiovascular Disease Mortality in Older Community-Dwelling Adults

Plasma levels of AGE were assayed in a population of Italian subjects to determine if a correlation between such levels and cardiovascular disease or other causes of mortality could be identified.

Study Population

The participants consisted of men and women, aged 65 and older, who participated in the Invecchiare in Chianti, “Aging in the Chianti Area” (InCHIANTI) study, a population-based study conducted in two small towns, Greve in Chianti and Bagno a Ripoli, in Tuscany, Italy. The study population and follow-up visits for data collection are described above.

Data Collection

Demographic information and information on smoking and medication use, alcohol intake, and education were collected using standardized questionnaires as described above. A trained geriatrician examined all participants, and diseases were ascertained according to standard as described above. Fasting plasma glucose was defined as normal, impaired, or diabetic based on a fasting plasma glucose of 99 mg/dL or less, 100 to 125 mg/dL, and greater than 125 mg/dL, respectively. The diagnosis of diabetes mellitus was based on the diagnostic algorithm, and of those who reported no diabetes mellitus, on a fasting plasma glucose of greater than 125 mg/dL. The diagnostic algorithm for the diagnosis of diabetes mellitus was based on the use of insulin, oral hypoglycemic agents, hemoglobin A1c, and a questionnaire administered to the primary care physician of the study participant.

Systolic and diastolic blood pressures were calculated from the mean of three measures taken using a standard mercury sphygmomanometer during the physical examination. Body mass index, MMSE, and renal insufficiency were characterized as above.

Laboratory Analysis

Blood samples were collected as noted above and CML was measured using a competitive enzyme-linked immunosorbent assay (ELISA) (AGE-CML ELISA, Microcoat, Bernried, Germany, under license to Synvista Therapeutics, Montvale, N.J.) as described above. The intra-assay variation was less than 5%.

Fasting blood glucose was determined according to an enzymatic colorimetric assay using a modified glucose oxidase-peroxidase method (Roche Diagnostics, GmbH, Mannheim, Germany) and a Roche-Hitachi 917 analyzer. Commercial enzymatic tests (Roche Diagnostics) were used for measuring serum total cholesterol, triglycerides, and high-density lipoprotein cholesterol (HDL-C) concentrations. Low-density lipoprotein cholesterol (LDL-C) was calculated using the Friedewald formula.

Statistical Analysis

Variables are reported as medians (25th, 75th percentiles) or as percentages. Plasma CML was divided into tertiles, and the cutoffs between tertiles were 314 and 396 ng/mL. Age and BMI were used as categorical variables because the relationship between age and BMI, respectively, with mortality was not linear. Characteristics of subjects according to their vital status were compared using Wilcoxon rank sum tests for continuous variables and chi-square tests for categorical variables. Cox proportional hazards models were used to examine the relationship between plasma CML and all-cause and CVD mortality over 6 years of follow-up. Variables that were significant in the univariate analyses were entered into the multivariate Cox proportional hazards models, except for CVD mortality, in which CVD were not included. Survival curves were compared using log-rank tests. The statistical program used was SAS (SAS Institute, Inc., Cary, N.C.).

Results

Plasma CML concentrations were measured at enrollment in 1,013 participants. During 6 years of follow-up, 227 (22.4%) of 1,013 participants died, of whom 105 died with CVD. The main causes of death were CVD (46.3%); cancer (26.1%), respiratory disease, including chronic obstructive pulmonary disease and pneumonia (10.1%); and other (17.1%). The cause of death was unknown for one participant. The vital status of all 1,013 participants was known for the 6 years of follow-up.

Demographic and other characteristics of participants who died from all causes or survived are shown in Table 1. Median plasma CML concentrations were significantly higher in adults who died from all causes than those who survived.

Participants who died from all causes were more likely to be older, male, less educated, and taking aspirin and to have a higher BMI; higher mean arterial pressure; abnormal fasting plasma glucose; lower total cholesterol, HDL-C, and LDL-C; a MMSE score less than 24; coronary heart disease, congestive heart failure; peripheral arterial disease; stroke; and renal insufficiency. There were no significant differences between participants who survived or died from all causes in smoking status, triglycerides, diabetes mellitus, or cancer.

The proportions of participants who died from all causes in the lower, middle, and upper tertiles of plasma CML were 18.6%, 19.2%, and 29.3%, respectively (P=0.001). Survival curves for all-cause mortality in participants in the highest tertile of plasma CML versus the lower two tertiles are shown in FIG. 1A.

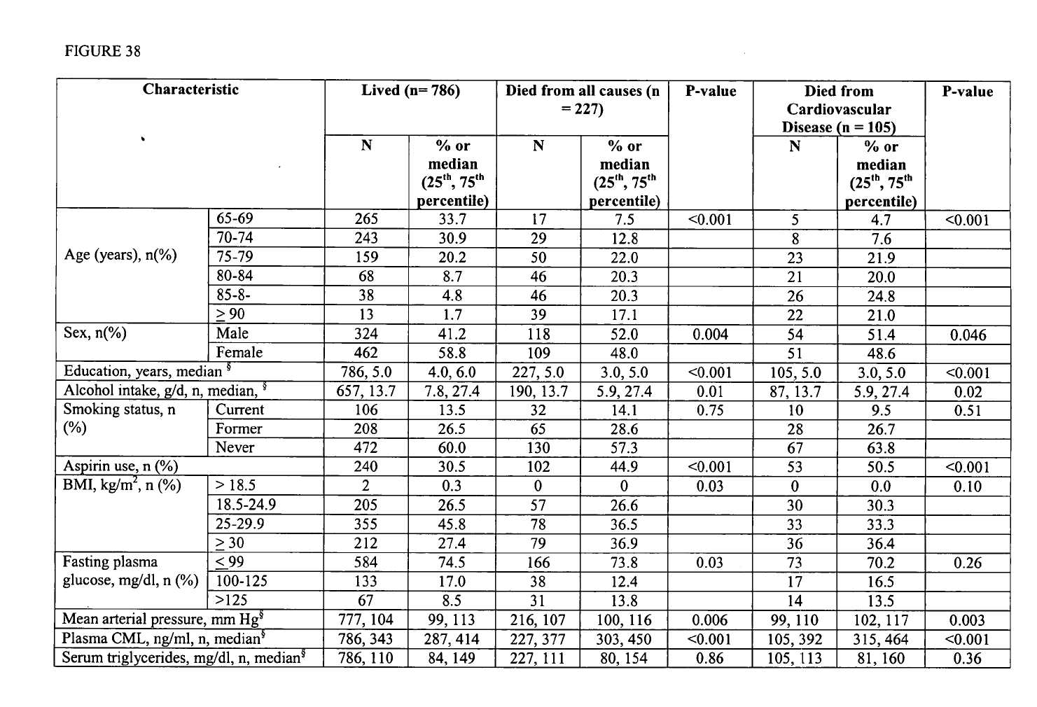

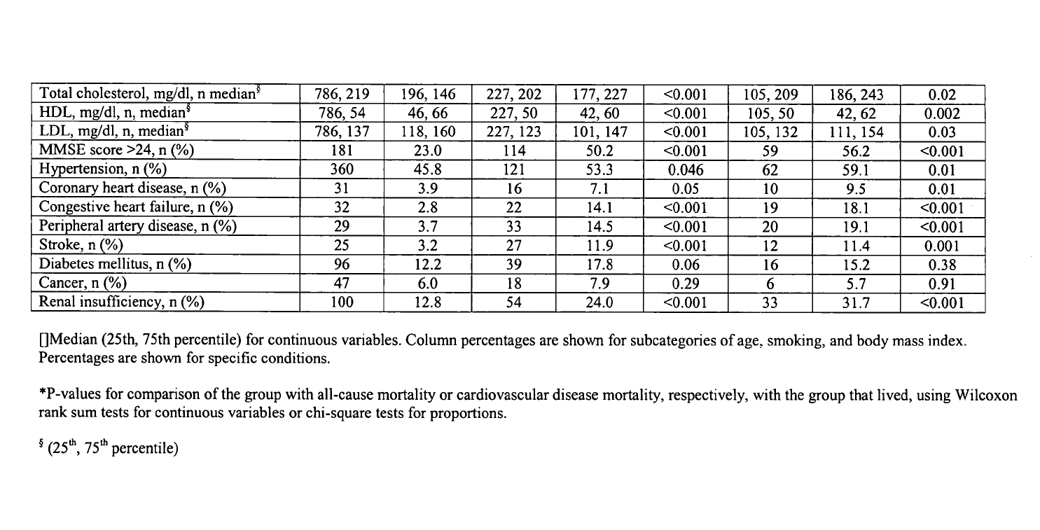

Demographic and other characteristics of adults who died from CVD or survived are shown in FIG. 38. Median plasma CML concentrations were significantly higher in adults who died from CVD compared to adults who survived. Adults who died from CVD were more likely to be older, male, less educated, and taking aspirin and have higher mean arterial pressure; abnormal fasting plasma glucose; lower total cholesterol, HDL-C, and LDL-C; a MMSE score less than 24; hypertension; coronary heart disease; congestive heart failure; peripheral arterial disease; stroke; and renal insufficiency. There were no significant differences between adults who survived or died from all causes in smoking status, BMI, triglycerides, diabetes mellitus, or cancer.

The proportions of participants who died from CVD in the lower, middle, and upper tertiles of plasma CML were 8.3%, 9.9%, and 17.3%, respectively (P=0.001). Survival curves for CVD mortality in adults with plasma CML in the highest tertile of plasma CML versus the lower two tertiles.

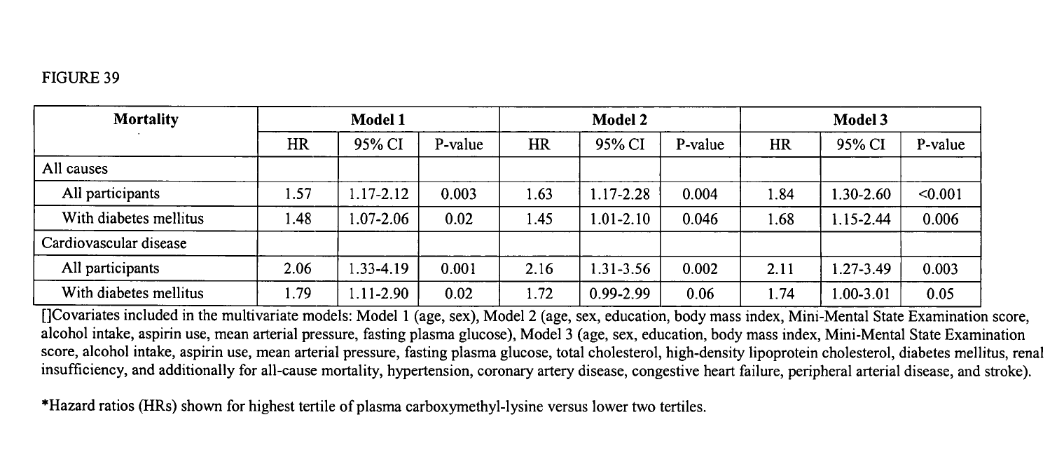

Multivariate Cox proportional hazards models were used to examine the relationship between plasma CML and all-cause and CVD mortality (FIG. 39). Plasma CML was an independent predictor of all-cause mortality in multivariate Cox proportional hazards models that adjusted for age and sex; additionally for education, aspirin use, BMI, MMSE, alcohol intake, mean arterial pressure, and fasting plasma glucose; and additionally for total cholesterol, HDL-C, diabetes mellitus, renal insufficiency, and CVD (hypertension, coronary artery disease, congestive heart failure, peripheral artery disease, and stroke) in all participants and after excluding participants with diabetes mellitus. No significant interactions were found between diabetes mellitus and plasma CML in multivariate Cox proportional hazards models for all-cause and CVD mortality.

Plasma CML was an independent predictor of CVD mortality in multivariate Cox proportional hazards models that adjusted for age and sex; additionally for education, aspirin use, BMI, MMSE, alcohol intake, mean arterial pressure, and fasting plasma glucose; and additionally for total cholesterol, HDL-C, diabetes, and renal insufficiency in all participants (FIG. 39). Plasma CML was an independent predictor of CVD mortality in participants without diabetes mellitus, adjusting for the same covariates above.

Plasma CML was not a significant predictor of non-CVD mortality in multivariate Cox proportional hazards models adjusted for age and sex (HR=1.38, 95%=CI 0.91-2.08, P=0.13); additionally for education, aspirin use, BMI, MMSE, alcohol intake, mean arterial pressure, and fasting plasma glucose (HR=1.48, 95% CI=0.90-2.28, P=0.13); and additionally for renal insufficiency and CVD (HR=1.48, 95% CI=0.93-2.37, P=0.10).

This study demonstrates that older community-dwelling men and women with high plasma CML are at greater risk of dying, especially from CVD. To the authors' knowledge, this is the first study to show that plasma CML is an independent predictor of all-cause and CVD mortality in older community-dwelling adults.

In this study, plasma CML was independently predictive of CVD mortality and all-cause mortality. The magnitude of the HRs for mortality was greater for CVD mortality than all-cause mortality, suggesting that high plasma CML may be more specifically involved in CVD mortality. In addition, plasma CML was not significantly predictive of non-CVD mortality. Although it is believed that the hyperglycemia associated with diabetes mellitus increases the generation of endogenous AGEs, the relationship between plasma CML and all-cause and CVD mortality was found in patients without diabetes mellitus. The association between plasma CML and all-cause and CVD mortality was nonlinear, with a threshold at the highest tertile. These findings suggest that there may be a critical threshold for plasma CML above which the risk of mortality increases greatly.

### Example 11

### Relationship of an Advanced Glycation End Product, Plasma Carboxymethyl-Lysine, with Slow Walking Speed in Older Adults: The InCHIANTI Study

We characterized the relationship between a plasma AGE, carboxymethyl-lysine (CML), and slow walking speed (lowest quintile of walking speed) in older adults.

Study Population

The study participants consisted of men and women, aged 65 and older, who participated in the Invecchiare in Chianti, “Aging in the Chianti Area” (InCHIANTI) study, conducted in two small towns in Tuscany, Italy. The population is described in the examples above.

Data Collection

Demographic information and information on smoking and medication use, alcohol intake, and education were collected using standardized questionnaires as described above. A trained geriatrician examined all participants, and diseases were ascertained according to standard as described above. In the 4-meter walking test, the participants were instructed to walk at their normal pace over a 4 meter distance, were repeated twice and the average time was used (Bandinelli et al. 2006). Participants were categorized as having slow walking speed if they were in the slowest quintile of walking speed, which in this population was <0.79 msec.

Blood samples were collected in the morning after a 12-h fast. Aliquots of serum and plasma were immediately obtained and stored at −80° C. The measure of plasma AGEs in this study was plasma carboxymethyl-lysine (CML). CML was measured using a competitive enzyme-linked immunosorbent assay (ELISA) (AGE-CML ELISA, Microcoat, Penzberg, Germany) as described above. The intra-assay and inter-assay coefficients of variation were both <5%.

Variables are reported as medians (25th, 75th percentiles) or as percentages. Plasma CML was divided into quartiles, and the cut-off at the highest quartile was 424 ng/mL. Age and BMI were used as categorical variables because the relationship between age and BMI, respectively, with slow walking speed was not linear. Logistic regression models were used to examine the relationship between plasma CML and other risk factors with slow walking speed. Covariates that were significant in univariate analyses were included in the final multivariate models. All analyses were performed using SAS (v. 9.1.3, SAS Institute, Inc., Cary, N.C.) with a statistical significance level set at P<0.05.

The demographic, anthropometric, and disease characteristics of participants with and without slow walking speed are shown in FIG. 39. Participants with slow walking speed were older and more likely to be female, obese, not currently smoking, with MMSE score <24, and with depression, hypertension, congestive heart failure, peripheral artery disease, stroke, diabetes, and renal insufficiency compared with participants without slow walking speed. Plasma CML concentrations were higher in participants with slow walking speed compared to those without slow walking speed. There were no significant differences in the proportion of angina or cancer among participants with and without slow walking speed.

Participants in the highest quartile of plasma CML had greater odds of slow walking speed in separate multivariate logistic regression models, adjusted for age and sex, and additionally for education, smoking, MMSE, and for chronic diseases, respectively. The association between plasma CML and slow walking speed remained significant in similar models after excluding participants with diabetes.

The present study shows that elevated plasma CML is independently associated with slow walking speed in older community-dwelling adults. To our knowledge, this is the first study to show an association between elevated AGEs and impaired physical performance in older, community-dwelling men and women.

In conclusion, older adults with elevated plasma CML, an advanced glycation end product, had greater risk of slow walking speed.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of diagnosing a subject as having, or having a propensity to develop an ageing related disease or disorder, the method comprising detecting carboxymethyl lysine (CML) in a subject sample, wherein an alteration in the level of CML relative to the level in a control sample indicates that the subject has or has a propensity to develop an ageing related disease or disorder.

2. The method of claim 1, further comprising determining the level of one or more receptors for advanced glycation endproducts in the sample.

3. The method of claim 1, wherein ageing related diseases are selected from the group consisting of: reduced kidney function, renal insufficiency, reduced skeletal muscle strength, sarcopenia, cardiovascular disease, cardiovascular disease-related death, and anemia.

4. The method of claim 1, wherein the level of CML is determined in an immunological assay.

5. The method of claim 1, wherein the subject is a human female.

6. The method of claim 1, wherein the sample is serum.

7. The method of claim 1, wherein the sample is a non-fasting blood sample, or a fasting blood sample.

8. The method of claim 1, wherein the method detects an analyte selected from the group consisting of increased serum CML, increased receptor for advanced glycation end products (RAGE) expression, and increased circulating RAGE (sRAGE) as compared to a control.

9. The method of claim 8, wherein detection of increased serum CML as compared to control and increased circulating RAGE as compared to control identifies the subject as having increased propensity to develop reduced glomerular filtration rate (GFR).

10. The method of claim 8, further comprising detecting serum carotenoids.

11. The method of claim 10, wherein increased serum CML or other AGE, and low serum carotenoids are associated with a subject having or having a propensity to develop poor grip strength and/or sarcopenia.

12. The method of claim 8, wherein detection of increased circulatory RAGE as compared to control identifies the subject as having an increased risk of cardiovascular death.

13. The method of claim 12, wherein detection of circulatory RAGE comprises measuring total sRAGE and endogenous secretory RAGE (esRAGE).

14. The method of claim 8, wherein detection of increased serum CML as compared to a control identifies the subject as having or having a propensity to develop renal insufficiency.

15. The method of claim 12, wherein detection of increased serum CML, sRAGE, and esRAGE as compared to a control identifies the subject as having an increased propensity to develop anemia.

16. A method of treating or preventing an ageing related disease or disorder in a subject, the method comprising administering to a subject in need thereof an effective amount of a composition that reduces the risk associated with an increased level of CML or one or more receptors for advanced glycation endproducts.

17. The method of claim 16, comprising administering to the subject an AGE-breaker or AGE inhibitor.

18. The method of claim 16, comprising imposing on the subject dietary restriction of AGE-containing foods.

19. The method of claim 18, wherein the dietary restrictions comprise reducing the intake of foods processed at high temperatures, deep fried, oven fried, grilled, or broiled.

20. The method of claim 16, further comprising increasing carotenoid intake.

21. The method of claim 16, wherein the method treats or prevents a condition selected from the group consisting of reduced kidney function, renal insufficiency, skeletal muscle strength, sarcopenia, cardiovascular disease, cardiovascular disease-related death, and anemia.

22. A method of monitoring a subject having an ageing related disease, the method comprising detecting carboxymethyl lysine (CML) in a subject sample, wherein an alteration in the level of CML relative to the level in a control sample indicates that the subject has or has a propensity to develop an ageing related disease or disorder.

23. The method of claim 22, further comprising determining the level of one or more receptors for advanced glycation endproducts in the sample.

24. The method of claim 22, wherein ageing related diseases are selected from the group consisting of: reduced kidney function, renal insufficiency, skeletal muscle strength, sarcopenia, cardiovascular disease, cardiovascular disease-related death, and anemia.

25. The method of claim 22, wherein the level of CML is determined in an immunological assay.

26. The method of claim 22, wherein the subject is a human female.

27. The method of claim 22, wherein said sample is serum, a non-fasting blood sample, or a fasting blood sample.

28. The method of claim 22, wherein the method detects an analyte selected from the group consisting of increased serum CML, increased RAGE expression and increased circulating RAGE.

29. The method of claim 22, wherein the method monitors efficacy or compliance with dietary restriction.

30. The method of claim 29, wherein reduction in CML levels is indicative of the efficacy of a dietary restriction.

31. The method of claim 29, wherein no reduction in CML levels is indicative of a need for treatment with an AGE breaker or AGE inhibitor.

32. A kit for the diagnosis of an ageing-related disease in a subject comprising a composition for detecting CML in a sample and directions for use of the kit.

33. The kit of claim 32, wherein said composition comprises an antibody that detects CML in an immunological assay.

34. A method of selecting a treatment regimen for a subject having, or having a propensity to develop an ageing related disease or disorder, the method comprising detecting carboxymethyl lysine (CML) in a subject sample, wherein an increase in the level of CML relative to the level in a control sample indicates that the subject should be treated to reduce AGE or RAGE levels.

35. The method of claim 34, further comprising determining the level of one or more receptors for advanced glycation endproducts in the sample.

36. The method of claim 34, wherein ageing related diseases are selected from the group consisting of: reduced kidney function, renal insufficiency, skeletal muscle strength, sarcopenia, cardiovascular disease, cardiovascular disease-related death, and anemia.

37. The method of claim 34, wherein the level of CML is determined in an immunological assay.

38. The method of claim 34, wherein the treatment comprises administering to the subject an AGE-breaker or AGE inhibitor.

39. The method of claim 34, wherein the treatment comprises imposing on the subject dietary restriction of AGE-containing foods.

40. The method of claim 39, wherein the dietary restrictions comprise reducing the intake of foods processed at high temperatures, deep fried, oven fried, grilled, or broiled.

41. The method of claim 39, further comprising increasing carotenoid intake.

42. The method of claim 34, wherein the method treats or prevents a condition selected from the group consisting of reduced kidney function, renal insufficiency, skeletal muscle strength, sarcopenia, cardiovascular disease, cardiovascular disease-related death, and anemia.

43. The method of claim 1, further comprising detecting a level of at least one biomarker selected from the group consisting of high density lipoprotein, low density lipoprotein, C reactive protein, total cholesterol, and triglycerides.
